(19) United States
(12) Patent Application Publication
(10) Pub. No.: US 2025/0011445 A1
(43) Pub. Date: Jan. 9, 2025

(54) COMBINATIONS OF ANTI-INFLAMMATORY AGENTS FOR TREATING ACUTE ORGAN FAILURE, ARDS, ORGANS FOR TRANSPLANTATION OR DISEASES CAUSED BY AN AIRWAY-TARGETING VIRUS

(71) Applicants: UCL BUSINESS LTD, London (GB); UNIVERSITÄT ZU KÖLN, Köln (DE); FUNDACIÓN INSTITUTO DE INVESTIGACIÓN SANITARIA ARAGÓN, Zaragoza (ES); FUNDACIÓN AGENCIA ARAGONESA PARA LA INVESTIGACIÓN Y EL DESARROLLO, Zaragoza (ES); UNIVERSIDAD DE ZARAGOZA, Zaragoza (ES)

(72) Inventors: Michael BERGMANN, London (GB); Diego DE MIGUEL SAMANIEGO, London (GB); Gianmaria LICCARDI, London (GB); Julian PARDO JIMENO, London (GB); Nieves PELTZER, London (GB); Maria SECRIER, London (GB); Lucia TARABORRELLI, London (GB); Henning WALCZAK, London (GB)

(21) Appl. No.: 18/030,974

(22) PCT Filed: Oct. 8, 2021
(86) PCT No.: PCT/EP2021/077942

§ 371 Date: Apr. 7, 2023

(30) Foreign Application Priority Data
Oct. 9, 2020 (GB) 2016058.6

(51) Int. Cl. C07K 16/28; A61K 31/167; A61K 31/192; A61K 31/275; A61K 31/573; A61K 31/616; A61K 39/00; A61K 45/06; A61P 31/14; C07K 16/24

(52) U.S. Cl. CPC C07K 16/2875; A61K 31/167; A61K 31/192; A61K 31/275; A61K 31/573; A61K 31/616; A61K 45/06; A61P 31/14; C07K 16/245; C07K 16/2866; A61K 2039/505

(57) ABSTRACT

The invention provides methods and means for the inhibition or neutralisation of death receptor/death ligand members of the tumour necrosis factor superfamil and TNF receptor superfamilies and/or the Toll-like receptor family, and/or NOD-like receptor and/or of the signalling induced via these receptors, preferably in combination with each other and/or with anti-inflammatory agents, in treating diseases such as Acute Respiratory Distress Syndrome (ARDS), such as that caused by airway-targeting viruses, such as SARS coronaviruses.

### CROSS-REFERENCE TO RELATED APPLICATIONS

This patent application is a national stage filing under 35 U.S.C. § 371 of International Application No. PCT/EP2021/077942, filed Oct. 8, 2021, which application claims the benefit of priority of United Kingdom Patent Application No. 2016058.6, filed Oct. 9, 2020, both of which are herein incorporated in their entirety.

### SEQUENCE LISTING

This application includes a sequence listing in txt format titled “134541.00035_ST25.txt”, which is 4,458 bytes in size and was created on Nov. 14, 2023. The sequence listing is electronically submitted with this application and is incorporated herein by reference in its entirety.

### TECHNICAL FIELD

The present invention relates generally to methods and materials for use in treating diseases of acute organ failure, including those caused by airway targeting viruses, such as Acute Respiratory Distress Syndrome, and related diseases.

### BACKGROUND TO THE INVENTION

Acute organ failure can result from a variety of diseases including acute respiratory distress syndrome (ARDS) or patho-physiologically related disease, including, but not limited to, infection- or non-infection-triggered sepsis, multi-organ failure and ischemia-reperfusion injury (IRI) and the end-stage of organ failure which is often a fibrotic disease. Fibrotic disease includes, but is not limited to, lung fibrosis such as idiopathic pulmonary fibrosis, fibrotic diseases of the skin such as scleroderma pigmentosum, and fibrotic diseases of the liver.

ARDS is the common denominator of severe acute lung failure requiring mechanical ventilation in critically ill patients (Matthay et al. 2019). The causes of ARDS are usually known and include pneumonia caused, e.g. by viruses, bacteria, fungi or gastric juice.

ARDS-inducing viruses include, but are not limited to, the common flu-causing influenza A and B viruses (IAV and IBV) (Thompson, Chambers, and Liu 2017; Ackermann et al. 2020; Kalil and Thomas 2019) and the corona viruses (CoVs) SARS-CoV(-1) (Thompson, Chambers, and Liu 2017), MERS-COV (Memish et al. 2020) and SARS coronavirus 2 (SARS-CoV-2) (Fan et al. 2020; Ackermann et al. 2020; Weiskopf et al. 2020).

The emergence of SARS-CoV-2 in the human population towards the end of 2019 has triggered an ongoing global pandemic of severe pneumonia-like disease designated as coronavirus-induced disease 2019 (COVID-19) which currently poses a major healthcare and economic threat globally. It has been reported that most COVID-19 patients die due to severe pulmonary consequences such as hypoxemic respiratory failure or ARDS (Bhatraju et al. 2020; Huang et al. 2020). Around 16% of severe COVID-19 patients develop ARDS and approx. 40%/3% of patients with severe COVID-19 require mechanical ventilation/ECMO support, respectively (Guan et al. 2020).

Alveolar epithelial cell death has been proposed to play a critical role in the pathogenesis of ARDS, including in COVID-19 patients (Zhou et al. 2020; Wu et al. 2020; Sauler, Bazan, and Lee 2019).

ARDS is the organ-damaging consequence of a process of uncontrolled inflammation induced by infectious or non-infectious agents. Regardless of the initial trigger, ARDS is characterized by an increased alveolar-capillary permeability to fluid, proteins, immune cells, including inflammatory immune cells such as, e.g. neutrophils, and red blood cells, resulting in their accumulation into the alveolar space, causing interstitial and alveolar oedema and diffuse alveolar damage (Matthay et al. 2019). Importantly, once ARDS is established, the disease acquires a unique pathophysiology with little if any dependence on the initial trigger. Therefore, current therapeutic approaches to ARDS mainly focus on the reduction of the already established inflammation and on the administration of supportive therapies such as lung protective ventilation, rather than on targeting the initial trigger (Griffiths et al. 2019) which, in the case of COVID-19 is infection by SARS-CoV-2 (Matthay, Aldrich, and Gotts 2020).

Despite the fact that a strong inflammatory component, including the so-called cytokine release syndrome (CRS), also referred to as “cytokine storm”, is evident in patients with ARDS, the sole use of anti-inflammatory drugs such as dexamethasone or prednisone rarely leads to resolution of the disease, even though such drugs have been shown to alleviate symptoms and reduce time of hospitalisation for certain COVID-19 patients (Horby et al. 2020).

CRS is a systemic inflammatory response that can be triggered by a variety of factors such as infections and certain drugs (Shimabukuro-Vornhagen et al. 2018; Murthy et al. 2019). CRS is a common side effect of chimeric antigen receptor (CAR) T cell therapy, in which patient T cells are engineered in vitro to express CAR molecules that recognize antigens on tumor cells, therefore activating tumor clearance when administered to the patient. These engineered T cells have been found to induce a very strong inflammatory response, developing severe CRS and secondary hemophagocytic lymphohistiocytosis (sHLH) which, in certain cases, causes ARDS and multi-organ failure (Teachey et al. 2016; Gauthier and Turtle 2018; Maude, Barrett, et al. 2014). Although the precise mechanism how CAR T cells induce severe CRS is still elusive, it appears to be linked to the rapid death of many malignant cells induced by the CAR T cells within a brief period following their infusion (Davila et al. 2014; Maude, Frey, et al. 2014; Lee et al. 2015; Park et al. 2018). Of note, T cells mainly induce cell death via death ligands and/or the perforin-granzyme system (Martínez-Lostao, Anel, and Pardo 2015). Whilst the majority of this cell death is thought to occur by apoptosis which has been regarded as an immunologically silent form of cell death, recent studies have unveiled the existence of a complex crosstalk between apoptosis and other pro-inflammatory forms of cell death, such as necroptosis and pyroptosis.

Importantly, we recently made the unexpected discovery that caspase-8-dependent cell death induced by death ligands such as TNF, LT-alpha, TRAIL and/or FasL, which had always been thought to be apoptotic, can be the causative for lethal inflammatory disease (Gurung et al. 2014; Gringhuis et al. 2012; Yu et al. 2019; Orning et al. 2018; Sarhan et al. 2018; Gerlach et al. 2011; Rickard et al. 2014; Kumari et al. 2014; Peltzer et al. 2018; Taraborrelli et al. 2018).

In line with this, a recent study demonstrated how CAR T cells can induce CRS by activating gasdermin E (GSDME), an executioner of pyroptosis, in a model of B cell acute lymphoblastic leukemia (Liu, Fang, et al. 2020). Interestingly, the authors found that the amount of perforin/granzyme B used by the T cells is critical for the activation of GSDME. Thus, in a scenario of excessive cell death, such as the one induced by CAR T cells or in an acute infection, the initial pro-inflammatory signal triggered would be sensed and amplified by monocyte-macrophages which release high amounts of IL-6, swiftly causing CRS, but importantly, as a consequence of the massive, nearly simultaneous induction of cell death caused by the action of the infused CAR T cells.

There are, at present, multiple clinical trials to identify effective treatments for patients with COVID-19, but only relatively few based on drugs which either act in an anti-viral or anti-inflammatory manner, having been approved for the treatment of patients with COVID-19 (https://opendata.ncats.nih.gov/covid19/).

Patients that require supplemental oxygen but are not on high-flow oxygen, non-invasive ventilation or mechanical ventilation have the option of utilising Remdesivir, a broad-spectrum antiviral drug. However, recently the benefit of treatment with Remdesivir for patients with COVID-19 has been put into question (Wang, Zhang, Du, et al. 2020).

Following the results of the RECOVERY trial (Horby et al. 2020), dexamethasone was recommended for up to 10 days in all patients and in its absence, prednisone, methylprednisolone or hydrocortisone were recommended as alternatives (Czock et al. 2005; Alhazzani et al. 2020; Wang, Jiang, et al. 2020; NIH 2020).

However, even if ARDS resolves, around 20% of the patients suffer from fibrotic remnants causing chronic lung dysfunctions. This apparently occurs as a consequence of replacement of perished normal tissue which underwent uncontrolled and untoward cell death by fibroblasts. Such a pathophysiological mechanism serves to explain why antimicrobial treatment does not exert a significant therapeutic effect in many patients once the process towards development of ARDS is already too far progressed or indeed ARDS is already established (Matthay et al. 2019).

In a related line of research, using an in-vivo model of decreased linear ubiquitination, which results in increased cell death, we previously showed that aberrant cell death which was, at least in part, mediated via Toll-like receptor 3 (TLR3) is causative for increased lung pathology following IAV infection (Zinngrebe et al. 2016).

Furthermore, we previously showed that cells can be sensitized to TLR3-mediated death by IL-24, a cytokine which by itself does not mediate cell death (Weiss et al. 2013). Thus, in many cases, the capacity of receptors to induce cell death depends on the action of cofactors which is required to enable these receptors to indeed be capable of inducing cell death, i.e. “to provide them with the license to kill”.

Providing novel treatments for use in treating diseases of acute organ failure, including those caused by airway targeting viruses, such as ARDS (whether induced by respiratory viral, bacterial or fungal infections, or induced independently of an infection) and related diseases would provide a useful contribution to the art.

### DISCLOSURE OF THE INVENTION

As explained herein, the present invention is based on the understanding that acute organ dysfunction such as ARDS is a consequence of a spiral of cell death inducing inflammation and inflammation causing further tissue damage which in turn causes more inflammatory cell death.

As explained herein, this vicious circle may be broken by the effective inhibition of
(i) the biochemical machinery which mediates and/or the factor(s) which initiate(s) or transmit(s) the cell death-causing inflammation,
(ii) the biochemical machinery which mediates and/or the factor(s) which initiate(s) or transmit(s) the inflammation that causes tissue damage/cell death, or, as the preferred way of treatment,
(iii) the concomitant combination of these two mechanisms, i.e. combining the effective inhibition of (i) with that of (ii).

As explained hereinafter, it has unexpectedly been found that whilst a lethal inflammation which is caused by aberrant cell death can be effectively prevented by prophylactic inhibition of cell death, once inflammation had been initiated and is ongoing, the most effective treatment requires combined inhibition of the inflammation-causing cell death and of the inflammation itself whereas either treatment alone only provided very limited therapeutic benefit (see e.g. FIG. 1 and Example 1).

Through our unravelling of the pathophysiological mechanism causative for ARDS we disclose herein methods and materials for attenuating its often severe acute and/or chronic consequences, and thereby provide novel treatments for ARDS and other diseases with acute organ failure.

The mechanisms proposed herein are consistent with models of respiratory virus-induced ARDS. For example, ARDS-causing murine models of infections by such viruses are characterized by an initial peak of viral load around day 5 post infection followed by a peak of tissue destruction caused by type I interferon (IFN)-dependent hyperinflammation and corresponding cell death (Hogner et al. 2013; Davidson et al. 2014), followed by pulmonary fibrosis (Lopez et al. 2009). It therefore appears that ARDS, rather than being solely a consequence of the direct tissue destruction through lytic infection of cells by the virus which initiates the pathophysiology, is indeed the consequence of a hyperinflammation that is driven by host-mediated aberrant cell death, most likely including of lung epithelial cells (Herold et al. 2012), but not necessarily limited thereto. Moreover, the model implies that lung fibrosis is a consequence of hyperinflammation. The model also suggests that type I IFNs are essential, or at least provide substantial support, for the CDI-CRS-ICD spiral discussed in the following section, and the tissue damage resulting therefrom.

It will therefore be understood that, through our novel understanding of the mechanisms underlying the pathophysiology of the spiral of cell death-induced inflammation (CDI) and inflammation in turn causing more cell death (ICD) and, consequently, increased tissue damage, especially with respect to the identification of the factors and pathways responsible for CDI and ICD, respectively, the present disclosure enables the provision of rational treatments for ARDS, other diseases with acute damage to the lung, but also other organs such as kidney, pancreas, liver, brain and/or heart, and/or the consequences thereof, e.g. fatigue syndromes, dementia, chronic heart disease, diabetes and/or fibrotic diseases, e.g. of lung, skin or liver.

The present invention is therefore based generally on the inhibition or neutralisation of death receptor/death ligand members of the tumour necrosis and TNF receptor (TNFR) superfamilies (i.e. “death ligands” and “death receptors”/ligands), and/or the Toll-like receptor (TLR) family, and/or NOD-like receptor (NLR) and/or of the signalling induced via these receptors, particularly the signalling towards cell death but also the non-cell-death-related, mostly gene-activatory signalling they can induce such as the induction of different cytokines (Kohase, Henriksen-DeStefano et al. 1986; Zhang, Lin et al. 1988; Shalaby, Waage et al. 1989; Cullen, Henry et al. 2013; Hartwig, Montinaro et al. 2017), each one alone or in combination with each other, and/or with anti-inflammatory agents, in treating diseases such as ARDS or other acute organ failure, caused, e.g. by airway-targeting viruses, such as SARS-CoV-2 but also other SARS coronaviruses, an influenza A virus (IAV), an influenza B virus (IBV) or by other microbial or non-microbial agents.

As explained above, the agents or combinations of agents described herein may also be employed more generally to treat diseases involving CRS or diseases characterised by tissue damage, including, but not limited to, tissue damage caused by sensitisation of transformed or non-transformed epithelial and/or non-epithelial cells to untoward death, whereby the sensitization and/or cell death induction can be independent of dependent on interferons (IFN), including type I, type II and/or type III IFNs.

Sustained cell death induction may arise in situations in which one or more than one cell death program is activated which may be the initial trigger of inflammation or, alternatively, serve to amplify an ongoing inflammation. The present inventors have previously noted in non-virus-induced in-vivo models that death receptor/ligand-mediated cell death induced via the TNF/LT-alpha-TNFR1, TRAIL-TRAIL-R, Fas-FasL (CD95/CD95L; the terms are used interchangeably) and/or TLR3-dsRNA systems, can be causative for lethal inflammatory disease in mice with genetic alterations in linear ubiquitination (Gerlach et al. 2011; Zinngrebe et al. 2016; Peltzer et al. 2018; Taraborrelli et al. 2018).

In separate work, a targeted analysis described hereinafter has revealed that the death ligands TRAIL, FasL, LT-alpha and TNF are all significantly upregulated in the lungs of SARS-CoV-MA15-infected mice (FIG. 2), a mouse-adapted version of SARS-CoV(-1) (Roberts et al. 2007). It is believed that the SARS-CoV-MA15 model serves as an adequate and suitable model for the diseases caused by SARS coronavirus infections of humans.

The disclosure herein provides evidence of a key involvement of cell death and inflammation induced by TRAIL and/or FasL, but also of related death ligands and the receptors they bind to, in the pathological cell death that occurs in the lungs of COVID-19 patients and subjects suffering from related diseases.

In some aspects, the present invention concerns the treatment of coronavirus infections, by one or more agents.

It has been reported that antibody-mediated inhibition of TRAIL was sufficient to increase survival of IAV-infected mice, with some authors attributing this effect to prevention of TRAIL-induced cell death (Davidson et al. 2014) and others to a non-cell-death-mediated mechanism which depended on downregulation of a specific ion pump on lung epithelial cells (Peteranderl et al. 2016). In a murine model of IAV infection, treatment with an anti-TRAIL antibody was also reported to reduce the bacterial load of Streptococcus pneumoniae when the antibody was administered simultaneously with the infectious agent (Ellis et al. 2015).

Other findings of the inventors in support of the present invention are described hereinafter and provide a well based rationale for therapeutic intervention by inhibition of the death ligand TRAIL with the intention of providing an effective treatment for COVID-19 patients by preventing alveolar epithelial cell death and the ensuing cytokine release syndrome (CRS) and inflammation which causes ARDS.

Based on the disclosure herein it is envisaged that inhibition of cell death induced by one or more death ligands can prevent a significant portion of the pathological cell death that is induced in COVID-19 patients.

Thus, in one aspect there is provided a method for treating a subject for a coronavirus induced disease, the method comprising administering to the subject a TRAIL-inhibiting agent.

Whilst these agents alone may be capable of providing therapeutic benefit to certain patients, on the basis of the disclosure herein, it is believed that the therapeutic benefit will be substantially increased when the cell death-inhibitory therapy is combined with an anti-inflammatory therapy which is discussed in detail hereinafter.

A “[ligand]-inhibiting agent” or “[ligand]-inhibitor” as used herein is understood, unless context demands otherwise, to encompass an agent which inhibits the respective ligand's effect, such as to neutralise the ligand and/or inhibit its cellular receptor(s) and/or to diminish their respective interactions and/or the activities resulting from these interactions (for example, ultimately, cell death, in the case of death-receptor ligands). Such agents may inhibit more than one ligand in this way.

Likewise, a “[receptor]-inhibiting agent” or “[receptor]-inhibitor” as used herein is understood, unless context demands otherwise, to encompass an agent which inhibits the respective receptor's effect, such as to neutralise the receptor and/or to inhibit its ligand(s) and/or diminish their respective interactions and/or the activities resulting from these interactions (for example, ultimately, cell death, in the case of death receptors). Such agents may inhibit the effect of more than one receptor in this way.

“Neutralises” in this context will be understood to mean modulates a biological activity of, either directly (for example by binding to the relevant target) or indirectly. As used herein, the term “biological activity” means any observable effect resulting from the interaction between the protein\receptor (binding partners). Non-limiting examples of biological activity in the context of the present invention include signalling and regulation of the genes discussed herein, e.g. those involved in cell death by apoptosis, necroptosis and/or pyroptosis or in activation of genes that lead to the expression of cytokines, including chemokines.

“Neutralises” does not imply complete inactivation. The modulation is generally inhibition i.e. a reduction or diminution in the relevant biological activity by comparison with the activity seen in the absence of the agent.

Neutralisation is typically achieved by (i) preventing or inhibiting the ligand from binding to the receptor; (ii) disrupting the receptor/ligand complex resulting from such binding; (iii) preventing or inhibiting the activity of factors and/or enzymes responsible for mediating the biological activity normally induced by the ligand-induced stimulation of the receptor(s).

Thus, in one aspect the invention provides a method for treating an individual for a coronavirus-induced disease, the method comprising administering to the individual an agent that neutralises TRAIL and/or a TRAIL-R which is optionally TRAIL-R1 and/or TRAIL-R2 and/or diminishes the interaction of TRAIL with TRAIL-R1 and/or TRAIL-R2 and/or the activity resulting from these interactions.

Optionally the coronavirus is selected from SARS-CoV, SARS-CoV-2 and MERS-COV and/or the coronavirus-induced disease is selected from COVID-19, SARS and MERS.

Preferably this therapy is used in combination with at least one anti-inflammatory drug.

In some aspects, the present invention concerns the treatment of influenza infections, by one or more agents.

In one aspect the invention provides a method for treating an individual for an influenza virus-induced disease, the method comprising administering to the individual a TRAIL-inhibiting agent that neutralises TRAIL and/or a TRAIL-R which is optionally TRAIL-R2 and/or diminishes TRAIL/TRAIL-R activity.

Optionally the virus is influenza A or B virus (IAV and IBV).

Optionally the method further comprises administering an anti-inflammatory agent as described below.

In some aspects, the present invention concerns the treatment of diseases of airway-targeting coronavirus infections, by combinations of agents.

In one aspect of the invention there is provided a method for treating a subject for a disease caused or induced by an airway-targeting virus,
the method comprising administering to the subject a combination treatment of at least 2 agents, the combination comprising:
(i) a FasL (CD95L)-inhibiting agent that neutralises CD95L and/or a receptor thereof and/or diminishes CD95/CD95L activity, and
(ii) a TRAIL-inhibiting agent that neutralises TRAIL and/or a TRAIL-R which is optionally TRAIL-R2 and/or diminishes TRAIL/TRAIL-R activity.

As explained herein, in some aspects, the present invention concerns the treatment of these and other diseases by other combinations of agents.

Thus, the invention provides a method for treating a subject for a disease selected from acute organ failure, ARDS, which is optionally induced by an airway-targeting virus, or a patho-physiologically related disease, or for treating an organ prior to or after transplantation, the method comprising administering to the subject or organ a combination treatment of at least 2 agents, the combination comprising:
(1) a TRAIL blocker, which is optionally a TRAIL-R2-Fc;
(2) a FasL (CD95L) blocker, which is optionally a Fas-Fc (Asunercept) or an anti-FasL antibody;
(3) a TWEAK inhibitor, e.g. FN14-Fc or an anti-TWEAK antibody
(4) A RIPK1 kinase inhibitor, e.g. GSK2982772
(5) A caspase inhibitor, e.g. emricasan.

Preferably these methods further comprise use of an anti-inflammatory agent.

As noted above, in some aspects, the present invention concerns the treatment of a variety of etiologically related diseases by combinations of agents including anti-inflammatory or similar agents.

The present inventors have noted that, apart from TRAIL (Lee et al. 2008; Bem et al. 2010), also soluble FasL (CD95L) was found to be increased in the BAL fluid of patients with ARDS and other types of lung injuries as compared to the BAL fluid of control individuals (Matute-Bello et al. 1999; Hashimoto et al. 2000; Kuwano et al. 2000; Albertine et al. 2002).

Activation of the Fas/FasL pathway was shown to be capable of inducing lung injury and fibrosis in vivo (Hagimoto et al. 1997; Matute-Bello, Liles, et al. 2001). Non-therapeutic pharmacologic FasL blockade or genetic loss of Fas or FasL were also previously shown to prevent bleomycin-, LPS and haemorrhagic shock-induced lung injury in vivo (Kuwano et al. 1999; Kitamura et al. 2001; Matute-Bello et al. 2004; Perl et al. 2007). Cell death induced by immune cell-derived FasL was found to be responsible for IAV-induced lethality in clAP2-deficient mice (Rodrigue-Gervais et al. 2014) and non-therapeutic pharmacologic inhibition of FasL with a recombinant Fas-Fc protein has been shown to significantly reduce IAV-induced lethality in mice (Fujikura et al. 2013).

However, the observed protection in the latter was only partial and, importantly, it was not deducible whether any therapeutic effect could be achieved if the Fas-Fc protein were to be given therapeutically rather than non-therapeutically, i.e. prophylactically, meaning either before or concomitantly with IAV infection. In summary, the existing results suggest that the Fas-FasL system contributes to lung pathology induced by respiratory viruses. Thus, one could conclude that certain COVID-19 patients may benefit from an agent that neutralises FasL and/or Fas, i.e. the cellular receptor of FasL, and/or diminishes the interaction of FasL with Fas and/or the activity resulting from this interaction, such as, e.g. a Fas-Fc protein such as Asunercept.

The present inventors have also analysed which cell types were enriched or decreased in the lungs of SARS-CoV-MA15-infected mice and how an increase or decrease in any of these cell types correlated with the presence of FasL mRNA in the lung of each individual SARS-CoV-MA15-infected or mock-infected mouse. The result of this analysis revealed a highly significant positive correlation of the increased presence of FasL mRNA with that of an increase in the presence of different types of lymphocytic, myeloid and granulocyte cells, such as NK, pDCs and activated neutrophils (FIG. 2). Intriguingly, the increased presence of FasL mRNA—and of the cell types that most highly express it—also highly significantly correlated with a significant decrease in the presence of epithelial, endothelial and stromal cells such as club cells, capillary cells and alveolar fibroblast, respectively in the lungs of SARS-CoV-MA15. Interestingly, a highly similar pattern of expression could be observed for TNF, LT-alpha and Fn14 mRNA (FIG. 2). Based thereupon, we conclude that inhibiting FasL, e.g. with a FasL-inhibiting antibody such as RNOK203 (Mochida Pharmaceuticals, patent EP0957166A4, (Nisihara et al. 2001; Poonia et al. 2009)) or with a soluble Fas-Fc protein such as Asunercept (DKFZ/Apogenix, patent EP1606318A2 (Boch et al. 2018; Wick et al. 2019), is likely to be capable of inhibiting or at least diminishing the death of some of the same human cell types in the lungs of COVID-19 patients whose diminution correlated with high presence of FasL in the lungs of SARS-CoV-MA15-infected mice.

On the basis of the disclosure herein, namely (i) that even when cell death is the genetically proven etiological cause of a lethal inflammatory disease, the therapeutic effect of solely inhibiting this cell death or solely inhibiting inflammation once the cell death-induced inflammation has reached a certain level, can be very limited (FIG. 1), and (ii) that inhibiting cell death and concomitantly inhibiting inflammation is highly synergistic in providing an almost curative therapeutic effect of a cell death-caused, otherwise lethal inflammatory disease, even when applied at a time when this inflammation is already well established (FIG. 1), it is credible that the therapeutic effect that can be achieved by the inhibition of FasL will only become appreciable in many patients, or be increased substantially, when FasL inhibition is combined with at least one additional cell death inhibitor (e.g. a TRAIL-inhibiting agent) and/or anti-inflammatory agent, as described below.

Accordingly, the methods described herein based on the use of inhibitors of cell death ligands or their receptors (or resultant activities) preferably further comprise administering an anti-inflammatory agent.

Thus, the invention provides a method for treating a subject for a disease caused or induced by an airway targeting virus, the method comprising administering to the subject a combination treatment of at least 2 agents, the combination comprising:
(a) one or more agents selected from the list consisting of:
   (i) a FasL-inhibiting agent, and/or
   (ii) a TRAIL-inhibiting agent,
(b) an anti-inflammatory agent.

It further provides a method of enhancing the therapeutic effectiveness of:
(a) one or more agents selected from the list consisting of:
   (i) a FasL-inhibiting agent, and/or
   (ii) a TRAIL-inhibiting agent, for treating a subject for a disease caused or induced by an airway targeting virus the method comprising administering to the subject
(b) an anti-inflammatory agent.

It further provides a method comprising administering to the subject a combination treatment of at least 3 agents, the combination comprising:
(a) one or more agents selected from the list consisting of:
   (i) a FasL-inhibiting agent, and/or
   (ii) a TRAIL-inhibiting agent;
(b) an anti-inflammatory agent which inhibits the activity of TWEAK, RIPK1, Caspase(s) or Gasdermin(s);
(c) a further anti-inflammatory agent selected from: an agent which inhibits IL-6; and/or IL-6R; and/or IL6/sIL6R heterodimer, which is optionally sgp130-Fc, or an agent which inhibits TNF, which is optionally Etanercept.

The present disclosure has implications beyond FasL together with TRAIL systems for the treatment of airway-targeting viruses.

Collectively, from the analysis of the mRNA expression data shown in FIG. 2 one may deduce which of the different cell types analysed express TRAIL, FasL, TNF and/or LT-alpha in the lungs of SARS-CoV-MA15-infected mice (FIG. 2). Importantly, it implies that one or several of these cell types may induce, or contribute to, the disappearance/diminution of one or several of the cell types whose presence negatively correlated with TRAIL, FasL, TNF and/or LT-alpha expression in the lungs of SARS-CoV-MA15-infected mice, most likely by inducing their untoward death or at least contributing thereto. Thus, we conclude that aberrantly increased expression of TRAIL by, e.g. plasma cells, pDCs, NK cells, classical monocytes and/or goblet cells in the lungs of SARS-CoV-MA15-infected mice contributes to, or is responsible for, the death of, e.g., fibroblasts, lipofibroblasts, myofibroblasts, club cells, vascular smooth muscle cells and/or airway smooth muscle cells in the lungs of these mice. Similarly, increased expression of FasL by e.g. NK cells, NKT cells, pDCs, plasma cells and CD4⁺/CD8⁺ T cells is responsible for, or contributes to, the death of, e.g., pericytes, club cells, vascular and airway smooth muscle cells and alveolar epithelial type II cells.

Furthermore, it is important to note that in both the IAV models described above, whereas a certain degree of protection was evident by non-therapeutic pharmacologic inhibition of either TRAIL or FasL, including the prevention of bacterial co-infection (Ellis et al. 2015; Fujikura et al. 2013), in neither case the protection was complete. As mentioned before, another shortcoming of these studies is that the inhibitors were given prophylactically, i.e. concomitantly with or before infection, rather than therapeutically, meaning following infection.

Separately, it has been shown that pharmacologic but non-therapeutic inhibition of apoptotic cell death by zIETD-fmk reduces aberrant cell death, inflammation and fibrosis in lung tissue in both, a bronchiolitis obliterans organizing pneumonia (BOOP) model and in an ARDS model induced by reovirus 1/L (R1/L) infection (Lopez et al. 2009). Yet, whilst genetic deficiency in the Fas-FasL system was sufficient to prevent cell death, inflammation and fibrosis in the R1/L-induced model of BOOP, this was not the case for the reovirus 1/L-induced model of ARDS (Lopez et al. 2009).

These results could be interpreted such that an inducer of aberrant cell death different from FasL were responsible for R1/L-induced ARDS or that FasL inhibition, at least when used a stand-alone treatment, would not be capable to afford therapeutic benefit to patients suffering from ARDS. The former is unlikely as both Fas and FasL were also found to be upregulated in the R1/L-induced model of ARDS (Lopez et al. 2009). It is therefore unlikely that the lung tissue damage which was completely prevented by the genetic inactivation of the Fas-FasL system in the R1/L-induced model of BOOP, would be independent of this system in the R1/L-induced model of ARDS.

Instead, when considered in the context of our previous results (Gerlach et al. 2011; Taraborrelli et al. 2018; Peltzer et al. 2018) and novel findings herein, the results of the above study (Lopez et al. 2009) may be interpreted as implying that in certain patients in whom the pathology may be more severe than in patients in whom the inhibition of FasL alone is sufficient to achieve a significant therapeutic effect (e.g. in patients with ARDS) it is necessary to also block one or more other death ligands, and optionally to further combine this with one or more additional cell death-inhibitory, anti-inflammatory and/or immune-suppressive drugs to achieve a significant therapeutic effect.

Thus, in one aspect there is provided a method for treating a subject for a disease selected from acute organ failure, acute respiratory distress syndrome (ARDS), which is optionally induced by an airway-targeting virus, or a patho-physiologically related disease, or for treating an organ prior to or after transplantation, the method comprising administering to the subject or organ a combination treatment of at least 2 agents, the combination comprising:
(a) one or more agents selected from the list consisting of:
   a death-ligand inhibiting agent; and/or
   an agent which is a RIPK1 kinase inhibitor or RIPK3 inhibitor; and/or
   an agent which is a caspase inhibitor; and/or
   an agent that inhibits inflammasome activation and/or gasdermin activation; and
(b) one or more anti-inflammatory agents, different to the agent in (a).

In some embodiments the ligand in (a) and/or its cognate receptor is selected from the TNF Receptor superfamily members shown in Table 1.

In some embodiments the agent (a) is a TRAIL-inhibiting agent.

In some embodiments the agent (a) is a FasL-inhibiting agent.

In some embodiments the ligand in (a) and its cognate receptor are selected from dsRNA/TLR3, LPS/TLR4, TL1A/DR3, cGas/STING.

In some embodiments the method comprises an agent (a) which inhibits the activity of Caspase-8, Caspase-10 or Caspase-8 and Caspase-10.

In some embodiments the method comprises use of an agent (a) which inhibits:
(i) both TRAIL and FasL;
(ii) TRAIL and the IL-6/sIL-6R heteromer;
(iii) FasL and the IL-6/sIL-6R heteromer, or
(iv) TRAIL, FasL and the IL-6/sIL-6R heteromer

Inhibitors may achieve multivalent inhibition of more than one target, for example by use of multispecific antibodies as described hereinafter.

In some embodiments the anti-inflammatory agent is an agent which inhibits the activity of one or more ligands from the TNF Receptor superfamily member shown in Table 1.

In some embodiments the anti-inflammatory agent is an agent which inhibits TWEAK or a TWEAK-R which is optionally CD266.

In some embodiments the anti-inflammatory agent is an agent which inhibits IL-6; and/or IL-6R; and/or IL6/sIL6R heterodimer, wherein optionally the agent is the trans-signalling inhibitor sgp130-Fc or an anti-IL-6 antibody or anti-IL-6R antibody which is optionally Tocilizumab.

In some embodiments the anti-inflammatory agent is an agent which inhibits the activity of a Caspase, most preferably Caspase-8; RIPK1 kinase or RIPK3 kinase; and/or MLKL;

In some embodiments the anti-inflammatory agent is an agent which inhibits IL-1alpha; and/or IL-1beta; IL-18, or the NLPR3 inflammasome and/or gasdermins. Examples include Anakinra, Tadekinig-alpha, Disulfiran, Tanilast and MAS825.

In some embodiments the anti-inflammatory agent is an agent which inhibits the activity of TWEAK, RIPK1, or Caspase.

In some embodiments the anti-inflammatory agent is an agent which inhibits TLR3, or neutralises a ligand of TLR3, optionally via the dsRNA/TLR3 interaction.

In some embodiments the anti-inflammatory agent is an agent which inhibits a target in Table 2.

In some embodiments the anti-inflammatory agent is a TNF- or TNF/LT-alpha-inhibiting agent.

In some embodiments the anti-inflammatory agent is an agent which inhibits IL-1alpha- and/or IL-1beta.

In some embodiments the anti-inflammatory agent is an agent which inhibits the activity of Caspase-1, Caspase-4/5 or Caspase-1 and Caspase-4/5.

In some embodiments the anti-inflammatory agent is an agent which inhibits the activity of Caspase-3, Caspase-7 or Caspase-3 and Caspase-7.

In some embodiments the anti-inflammatory agent is an agent which inhibits the activity of RIPK1 kinase or RIPK3 kinase.

In some embodiments the anti-inflammatory agent is an inhibitor of the IFN-alpha Receptor, which is optionally Anifrolumab.

In some embodiments the anti-inflammatory agent is an inhibitor of MLKL.

In some embodiments the anti-inflammatory agent is nonsteroidal anti-inflammatory drug (NSAID) optionally selected from aspirin, inhibitors of COX-1 and/or COX-2, ibuprofen, paracetamol.

In some embodiments the anti-inflammatory agent is steroidal anti-inflammatory drug (SAID), optionally selected from triamcinolone, prednisone, prednisolone, methyl-prednisolone, dexamethasone, hydrocortisone, cortisol and/or related corticosteroids.

In some embodiments the anti-inflammatory agent is selected from Table 2.

In some embodiments the combination comprises a FasL-inhibiting agent (e.g. Fas-Fc, or other inhibitor listed in Table 2) and a TNF, LT-alpha or TNF/LT-alpha blocker (e.g. TNFR2-Fc, or other inhibitor listed in Table 2).

In some embodiments the combination comprises a TNF, LT-alpha or TNF/LT-alpha blocker (e.g. TNFR2-Fc, or other inhibitor listed in Table 2) and an NLRP3 (also know as NALP3) inhibitor (e.g. MCC950, or other inhibitor listed in Table 2).

In some embodiments the combination comprises a TNF, LT-alpha or TNF/LT-alpha blocker (e.g. TNFR2-Fc, or other inhibitor listed in Table 2) and dexamethasone (or other steroidal anti-inflammatory drug).

In some embodiments the agent in (a) is selected from or comprises:
(1) a TRAIL-inhibiting agent;
(2) a FasL-inhibiting agent;
(3) a TWEAK inhibiting agent;
(4) A RIPK1 kinase inhibitor;
(5) A caspase inhibitor,
   and the agent in (b) is selected from or comprises:
(6) a nonsteroidal anti-inflammatory drug (NSAID) and/or a steroidal anti-inflammatory drug (SAID);
(7) an agent which inhibits IL-6; and/or IL-6R; and/or IL6/sIL6R heterodimer, wherein optionally the agent is the trans-signalling inhibitor sgp130-Fc;
(8) a TNF, LT-alpha or a TNF/LT-alpha blocker.

In some embodiments the combination comprises one agent selected from (1)-(5) and one agent selected from (6)-(8).

In some embodiments the combination comprises one agent selected from (1), (2), (3), (4) and/or (5), combined with (6), with or without (7) and/or (8).

In some embodiments the combination comprises one agent selected from (1), (2), (3), (4) and/or (5), combined with (7), with or without (8).

In some embodiments the combination comprises one agent selected from (1), (2), (3), (4) and/or (5), combined with (8).

In some embodiments the combination comprises one agent selected from (1), (2), (3) and/or (4), combined with (6), which is optionally a SAID, (7) and/or (8) which is optionally a TNFR2-Fc fusion which is optionally Enbrel/Etanercept.

In some embodiments the combination comprises one agent selected from (1) and/or (2), combined with (6) as a SAID, (7) as sgp130-Fc and/or (8) which is optionally a TNFR2-Fc fusion which is optionally Enbrel/Etanercept.

In some embodiments the combination comprises one agent selected from (1) and/or (2), combined (6), as a SAID and/or (7) as sgp130-Fc.

In some embodiments the combination comprises a TRAIL-inhibiting agent, a FasL-inhibiting agent; a TNF- and/or TNF/LT-alpha-inhibiting agent.

In some embodiments the combination comprises a TRAIL-inhibiting agent and an inhibitor of type I and/or type II IFN signalling, which is optionally an inhibitor of a JAK1/2 kinase.

In some embodiments the combination comprises a TRAIL-inhibiting agent and an inhibitor of the IFN-alpha Receptor, which is optionally Anifrolumab.

In some embodiments the combination comprises a TRAIL-inhibiting agent and an inhibitor of blood DC antigen 2 (BDCA2), which is optionally BIIB059.

In some embodiments the combination comprises Fas-Fc and sgp130-Fc and a further anti-inflammatory agent, which is optionally methyl-prednisolone or dexamethasone.

In some embodiments the combination comprises a TRAIL-R2-Fc and sgp130-Fc and a further anti-inflammatory agent, which is optionally methyl-prednisolone or dexamethasone.

In some embodiments the combination comprises a TRAIL-R2-Fc, Fas-Fc and sgp130-Fc and a further anti-inflammatory agent, which is optionally methyl-prednisolone, prednisone or dexamethasone.

In some embodiments, if the agent is a RIPK1 kinase inhibitor, it is GSK2982772 and/or if the agent is a caspase inhibitor it is emricasan and/or if the agent is a TWEAK inhibitor it is FN14-Fc or an anti-TWEAK antibody and/or if the agent is a TNF, LT-alpha or TNF/LT-alpha blocker, it is Remicade, Humira, Baminercept, Pateclizumab or Enbrel/Etanercept.

As explained above, the present invention has utility in the treatment of a variety of diseases.

These include acute organ failure, ARDS, or a patho-physiologically related diseases.

The ARDS may result from an airway-targeting virus e.g. SARS-CoV(-1), MERS-COV, SARS-CoV-2, IAV, IBV, a reovirus, a rhinovirus.

The ARDS may be caused by bacterial or fungal infection.

The ARDS may be caused independently of an infectious agent e.g. by acid reflux.

The methods further have utility in treating an organ prior to or after transplantation,

The methods further have utility in treating other diseases caused or induced by airway-targeting viruses, bacteria or fungi.

However, it is understood in the light of the disclosure herein that the combinations of agents described herein may also be employed more generally in diseases involving cytokine release syndrome (CRS), or being characterised by type 1 and/or type 2 and/or type 3 IFN-mediated sensitisation of non-transformed cells to death ligand-induced cell death and/or type 1 and/or type 2 and/or type 3 IFN-mediated induction of death ligand expression.

For example, the combinations may be used to treat ARDS induced by other viruses and/or non-viral infections.

For example, the combinations may be used to treat other viral diseases in which hyperinflammation is indicated, e.g. Ebola, etc. or resulting from e.g. sepsis or other serious inflammatory diseases or, the failure of other organs including multi-organ failure.

Thus, any aspect or embodiments of the invention concerning diseases such as organ failure, ARDS, or caused or induced by airway-targeting viruses should be understood to apply mutatis mutandis to the other diseases described herein, particularly patho-physiologically related diseases or other aetiologically related diseases.

For example, inflammatory diseases.

For example, lymphopenia.

For example, acute organ failure caused by ARDS or other diseases with acute damage to the lung.

For example, ischemia reperfusion injury which may cause acute organ failure.

For example, infection- or non-infection-triggered sepsis and septic shock which may cause acute organ failure.

The organ is not limited to the lung, but may, for example, be the kidney, pancreas, liver, brain or heart.

The disease may be one which is consequential to the above e.g. organ damage. For example, fatigue syndromes, dementia, chronic heart disease, diabetes and/or fibrotic diseases, e.g. of lung, skin or liver.

Fibrotic diseases include, but are not limited to, lung fibrosis such as idiopathic pulmonary fibrosis, fibrotic diseases of the skin such as scleroderma pigmentosum, and fibrotic diseases of the liver.

For example, the combination agents may be used in organ failure associated with severe sepsis and septic shock. Organ failure of the liver is diagnosed by elevated liver enzymes and the inability to maintain adequate glucose levels.

For example, the combination agents may be used in organ failure following an ischemia reperfusion injury (IRI) following revascularization of ischemic tissue by vascular bypass or endovascular means such as balloon dilation and/or endoluminal stents.

For example, the combination agents may be used in organ damage/failure following ischemia reperfusion injury or following solid organ transplantation such as that of the kidney, heart, liver, lung and/or pancreas.

Given the disclosure above that concomitant inhibition of cell death and inflammation acts synergistically, it is proposed that the combination of, e.g. an agent that combines the inhibition of TRAIL or FasL or the cell death they induce (or indeed of an agent or agents that combine the inhibition of these two ligands or the cell death they induce), would be rendered significantly more efficacious when combined with one or more anti-inflammatory agents.

In aspects of the invention the therapeutic benefit of the CD95-inhibiting agent and/or TRAIL-inhibiting agent can be increased further, by combining these agents causing cell death-inhibition as described above with anti-inflammatory agents.

Thus, in one aspect the invention provides a method for treating an individual for a disease caused or induced by an airway-targeting virus,
the method comprising administering to the individual a combination treatment of at least 2 agents, the combination comprising:
(a) one or more agents which inhibits a death ligand selected from the list consisting of:
   (i) a Fas ligand (FasL)-inhibiting agent that neutralises FasL and/or Fas, i.e. the cellular receptor of FasL, and/or diminishes the interaction of FasL with Fas and/or the activity resulting from this interaction, and/or
   (ii) a TRAIL-inhibiting agent that neutralises TRAIL and/or a TRAIL-R which is optionally TRAIL-R1 and/or TRAIL-R2 and/or diminishes the interaction of TRAIL with TRAIL-R1 and/or TRAIL-R2 and/or the activity resulting from these interactions,
(b) an anti-inflammatory agent.

In a related aspect there is provided a method of enhancing the therapeutic effectiveness of:
(a) one or more agents which inhibit a death ligand selected from the list consisting of:
   (i) a FasL-inhibiting agent that neutralises FasL and/or Fas, i.e. the cellular receptor of FasL, and/or diminishes the interaction of FasL with Fas and/or the activity resulting from this interaction, and/or
   (ii) a TRAIL-inhibiting agent that neutralises TRAIL and/or a TRAIL-R which is optionally TRAIL-R1 and/or TRAIL-R2 and/or diminishes the interaction of TRAIL with TRAIL-R1 and/or TRAIL-R2 and/or the activity resulting from these interactions, for treating an individual for a disease caused or induced by an airway-targeting virus the method comprising administering to the subject:
(b) an anti-inflammatory agent.

Such combinations may be preferred when the disease has reached a stage of progression at which cell death inhibition alone does not afford significant therapeutic benefit, possibly due to the exacerbated inflammatory consequences of prolonged excessive cell death.

Croft and Siegel (Croft and Siegel 2017) discuss the potential of certain members of the TNF superfamily (TNFSF) as targets for future therapy of e.g. rheumatic diseases. They note TNFSF members initiate several processes, including immune activation, tissue inflammatory responses and cell death or suppression. In relation to blocking tissue inflammation, for example in patients with RA who were unresponsive to TNF blockers, there is a particular emphasis on neutralising TWEAK and LIGHT members of the TNFSF, in addition to TNF (page 229).

WO2019/141862 (Walczak, Taraborrelli, and Peltzer 2018) describes novel treatments (methods, uses and compositions) for treating inflammatory disease based on administering to the subject a combination of at least three agents targeting multiple death-receptor inducing systems, the combination comprising: (1) a first agent that neutralises the receptor TNFR1 or a ligand thereof; and (2) a second agent that neutralises either of: (2a) TRAIL-R, or a ligand thereof; or (2b) CD95, or a ligand thereof; and: (3) a third agent that neutralises any of: (3a) TLR3, or TLR4, or a ligand of either; or (3b) a further, different, receptor which is a TNF Receptor superfamily member shown in Table 1, or a ligand thereof; (3c) Caspase; (3d) RIPK1.

Such combinations for the treatment of inflammatory disease do not form part of the present invention.

Wurzer et al. (Wurzer et al. 2004) show that inhibiting FasL and TRAIL reduces release of influenza virus from infected cells, concluding that blocking TRAIL and/or FasL could be therapeutically useful in the treatment of influenza infections.

In several studies, Matute Bello et al. (Matute-Bello et al. 2004; Matute-Bello, Winn, et al. 2001; Matute-Bello et al. 1999; Matute-Bello, Liles, et al. 2001) show that FasL is involved in epithelial and alveolar cell apoptosis, in animal models but also in humans with ARDS. Moreover, functional deficiency of the FasL/Fas system reduced neutrophilic inflammation in the lungs in a model of LPS-induced lung inflammation in mice, demonstrating a pro-inflammatory role for FasL in lung infection (Matute-Bello et al. 2004).

As explained herein the combination therapeutics disclosed have particular utility in treating acute organ failure, such as sepsis-induced organ failure, including multi-organ failure and/or failure of the kidney and/or the liver, which failures are believed to depend on similar pathophysiological mechanisms.

The liver is a central organ in the inflammatory response. Liver failure severely aggravates severe sepsis and septic shock. Cells in the liver are known to be capable of expressing high levels of death receptors and their respective corresponding ligands such as Fas and FasL and/or TRAIL receptors 1 and 2 and TRAIL, especially in the inflamed liver.

Furthermore cytokines, especially type I and Type II IFNs, have been shown to significantly upregulate the expression of death ligands by different types of immune cells, including different types of T cells but also on natural killer (NK) cells, NKT cells and different types of dendritic cells (Chawla-Sarkar, Leaman, and Borden 2001; Griffith et al. 1999; Chow, Fang, and Yee 2000; Ruiz-Ruiz 2000; Sedger et al. 1999; Shin et al. 2001; Katsikis et al. 1997; Katsikis et al. 1996).

Importantly, sepsis and septic shock are known to often coincide with lymphopenia. It is likely that pathological exacerbation of activation-induced cell death (AICD), the natural means of lymphocyte regulation, is involved in, if not responsible for, lymphopenia. AICD has been known for a long time to be induced by Fas-FasL interaction (Dhein et al. 1995) and to be exacerbated during certain virus-induced lymphopenia such as in human immunodeficiency virus (HIV)-induced acquired immune deficiency syndrome (AIDS), partly because of aberrantly increased FasL-induced AICD but also in part because of increased TRAIL-induced lymphocyte death (Badley et al. 1998; Katsikis et al. 1997; Katsikis et al. 1996).

Organ dysfunction can also be caused by ischemia reperfusion injury (IRI). It has been shown that untoward cell death is a major player in IRI in several different organs (Linkermann et al. 2012; Gottlieb 2011; Noda et al. 1998). This untoward cell death, apart from itself causing damage to the tissue, results in hyperinflammation causing further tissue damage, which may be through programmed forms of cell death triggered by this hyperinflammation (e.g. through the activation of inflammasomes, i.e. by pyroptosis but also by, e.g. apoptosis, necroptosis and/or ferroptosis), with the consequence of further increasing organ damage.

In the light of the present disclosure it is credible that inhibition of uncontrolled cell death in combination with anti-inflammatory and/or immunosuppressive drugs could be beneficial therapeutically not only in the treatment of ARDS etc, but also in the treatment of other pathologies with acute organ failure, including sepsis-induced or sepsis-associated organ failure as well as IRI of organs including, but not limited to, heart, brain, kidney, pancreas, liver and lung.

Relatedly, in the case of transplantation of a solid organ, including, but not limited to transplantation of the heart, kidney, liver, pancreas, and/or lung, the associated IRI, in addition to damaging the organ and diminishing organ performance following transplantation, appears to be immunogenic and to drive organ rejection (Dashkevich et al. 2016; Nakamura, Kageyama, and Kupiec-Weglinski 2019).

Thus, in the light of the present disclosure, in a transplantation setting, inhibition, diminution or attenuation of untoward cell death or its conversion from a pro-inflammatory, possibly immunogenic, to a non-inflammatory and/or non-immunogenic form of cell death, would be highly desirable.

This may be both in an in vivo and ex vivo setting, prior to transplantation, as organs often need to be preserved for a prolonged period of time following their explantation from the donor before they can be implanted into the recipient.

In one embodiment the coronavirus induced disease is ARDS.

At least 7 coronaviruses are known to cause disease in humans.

Four of the 7 coronaviruses most frequently cause symptoms of the “common cold”.

Coronaviruses 229E and OC43 cause the common cold; the serotypes NL63 and HUK1 have also been associated with the common cold. Rarely, severe lower respiratory tract infections, including pneumonia, can occur, primarily in infants, older people, and the immunocompromised.

Three of the 7 coronaviruses cause much more severe, and sometimes fatal, respiratory infections in humans:
SARS-CoV-2 is a novel coronavirus identified as the cause of coronavirus disease 2019 (COVID-19) that began in Wuhan, China in late 2019 and has since spread worldwide.
MERS-COV was identified in 2012 as the cause of Middle East respiratory syndrome (MERS).
SARS-CoV was identified in 2002 as the cause of an outbreak of severe acute respiratory syndrome (SARS).

In one embodiment the coronavirus is selected from SARS-CoV(-1), SARS-CoV-2 and MERS-COV.

In one embodiment the coronavirus-induced disease is selected from COVID-19, SARS and MERS. However, based on the mechanisms described herein, the present invention will also have utility for potential future strains or outbreaks.

In one embodiment the airway targeting virus induces type 1 and/or type 2 and/or type 3 interferons leading to hyperinflammation as described herein.

In one embodiment the airway targeting virus induces ARDS.

ARDS and its causative agents have recently been discussed by Zemans et al. and by Matthay et al. (Zemans and Matthay 2017; Matthay et al. 2019), the disclosure of which is herein incorporated by cross reference. Viral pathogens associated with ARDS include influenza A viruses, coronaviruses, varicella zoster viruses, respiratory syncytial virus (RSV), rhinoviruses, parainfluenza, adenoviruses, and human metapneumovirus (hMPV) (Bauer et al. 2006; Nye, Whitley, and Kong 2016).

Thus, the airway-targeting virus may be any of these ARDS-related viruses. In one embodiment the airway-targeting virus is an influenza or a coronavirus.

In one embodiment the single or combination therapeutics described herein are used to treat a disease which is virus-induced, wherein the virus is influenza A or B virus (IAV and IBV) or any other influenza virus that infects humans or animals, e.g., but not limited to, cattle or pigs, or a coronavirus, e.g. SARS-CoV, MERS-COV, SARS-CoV-2 or other SARS coronavirus which infects humans and/or animals, e.g., but not limited to, cattle or pigs.

Methods described herein may comprise administering to a subject in need of such treatment a “therapeutically effective” amount of agents that decrease the biological activity resulting from activation of the death ligand(s) or receptor(s). In this embodiment this agent is a cell death inhibitor.

In one embodiment of the present invention the anti-inflammatory agent is an agent which inhibits IL-6; and/or IL-6R; and/or the IL6/sIL6R heterodimer. Optionally this may be, e.g. an antibody that binds to and neutralises IL-6 (e.g. olokizumab), an antibody that binds to IL-6R and prevents the IL-6-induced activation of signalling via IL-6R and its gp130 co-receptor (e.g. Tocilizumab), or, alternatively, a soluble form of gp130, e.g. a fusion protein between soluble gp130 and, e.g. an Fc portion of an antibody, e.g. of human lgG1, e.g. as in the case of the soluble gp130-Fc protein Olamkicept, a specific inhibitor of IL-6/sIL6R trans-signalling (Rose-John 2018).

IL-6 signalling is mediated by either the classical pathway or via trans-signalling (Rose-John 2018). In classical IL-6 signalling, the binding of IL-6 to the IL-6 receptor (IL-6R) induces signalling via gp130, with the latter being ubiquitously expressed whereas IL-6R is only expressed on a limited number of cell types including hepatocytes, certain other epithelial cells and selected types of leukocytes. Whilst IL-6 signalling through the classical pathway leads to intestinal cell proliferation and regeneration, inhibition of epithelial apoptosis, hepatic acute phase reaction and defence against bacterial infections, IL-6 trans-signalling induces the recruitment of mononuclear cells, stimulation of endothelial and smooth muscle cells and inhibition of T-cell apoptosis and differentiation.

Correspondingly, inhibition of IL-6R inhibits inflammation on the expense of bacterial infections and intestinal side effects. By contrast, inhibition of IL-6 trans-signalling via gp130 was shown to be effective at inhibiting pathogenic inflammation, including in septic shock (Greenhill et al. 2011; Barkhausen et al. 2011), atherosclerosis (Schuett et al. 2012), intestinal inflammation (Atreya et al. 2000), pancreatitis-induced lung failure (Zhang et al. 2013) and cancer-associated inflammation (Becker et al. 2004), importantly without facilitating bacterial infection. IL-6 trans-signalling therefore appears to play a major role in the pathology of acute inflammation, including CRS, but also in chronic inflammation.

For these reasons, in the light of the present disclosure, a preferred treatment of the invention is based on blockade of IL-6 trans-signalling, e.g. with sgp130-Fc (Olamkicept), as an effective anti-inflammatory drug in the treatment of the diseases described herein when synergised with cell death inhibitors, (e.g. inhibitors of TRAIL, FasL, TNF, and/or LT-alpha or the cell death they induce) in providing effective treatment for diseases caused by viruses (e.g. COVID-19), bacteria, ischemia reperfusion or other diseases including other inflammatory diseases.

In one embodiment of the present invention the anti-inflammatory agent is an agent which inhibits inflammasome activation.

The innate immune signalling receptor NLRP3 (NOD-, LRR-, and pyrin domain-containing 3) inflammasome leads to the release of inflammatory cytokines such as interleukin 1β (IL-1β) and IL18 (Swanson, Deng, and Ting 2019). The canonical NLRP3 inflammasome is a death platform that activates caspase-1 which in turn cleaves and activates Gasdermin D inducing disruption of the plasma membrane and release of inflammatory cytokines (Swanson, Deng, and Ting 2019). Consistent with this cytokine-mediated immunopathology, elevated levels of IL-1B, IL-6, and TNF have been observed in the broncho-alveolar lavage and plasma of ARDS patients (Han and Mallampalli 2015).

The SARS-CoV genome encodes 3 ion channel proteins: E, open reading frame 3a (ORF3a), and ORF8a in which E and ORF3a are required for both replication and virulence (DeDiego et al. 2014). In addition to the canonical NLRP3 activation pathway by PAMPs and DAMPs, the E, 3a, and 8b proteins of SARS-CoV function as NLRP3 agonists (Freeman and Swartz 2020). Given the CRS that occurs in COVID-19 patients, in the context of the present invention it is believed that targeting IL-1B or any of the inflammasome intermediates may reduce the inflammatory signalling that mediates lung injury, ARDS and mortality.

Production of the mature forms of the cytokines released by means of inflammasome activation is also regulated by other death receptor systems, for example TNF. In addition, pro-inflammatory cytokines can be released by the activation of non-canonical inflammasomes, such as, e.g. the NLRC4 inflammasome, or by the activation other Gasdermin proteins induced by alternative cell death pathways, e.g. caspase-8-mediated caspase-3 activation (Orning, Lien, and Fitzgerald 2019).

In the light of the disclosure herein it is proposed that in order to achieve maximal therapeutic benefit, inflammasome inhibition (including that of the canonical NLRP3 inflammasome) should be effected with concomitant inhibition of other inflammatory or cell death mediators.

Thus the invention further provides therapies in which inflammasome inhibitors are combined with one or more cell death inhibitor(s) including, but not limited to, inhibitors of TRAIL, FasL, LT-alpha and/or TNF, and/or with one or more anti-inflammatory agent(s).

Example inflammasome inhibitors include those known in the art (see e.g. reviews by Freeman and Swartz 2020; Ji et al. 2020; Mangan et al. 2018). Further example include the NLRP3 inhibitors MAS825 (Novartis) and MCC950 (commercially available).

In one embodiment the anti-inflammatory agent is an agent which inhibits the activity of a different receptor of the TNF Receptor superfamily member shown in Table 1, or one or more ligands thereof.

TABLE 1
TNF Receptor superfamily members and corresponding cognate ligands

| Member | Synonyms | Gene | Ligand(s) |
|---|---|---|---|
| Tumor necrosis factor | TNFR1, CD120a | TNFRSF1A | TNF (also |
| (TNF) receptor (TNFR) 1 | | | known as TNF- |
| | | | alpha) |
| | | | Lymphotoxin |
| | | | (LT)- alpha (also |
| | | | known as TNF- |
| | | | beta) |
| Lymphotoxin beta | LTBR, CD18 | LTBR | LT-alpha, LT- |
| receptor | | | beta |
| | | | LIGHT |
| Fas | APO-1, CD95 | APT1 | FasL (also |
| | | | known as CD95 |
| | | | or APO-1L) |
| TRAIL-R1 | Death receptor 4, | TNFRSF10A | TRAIL (also |
| | Apo-2, CD261 | | known as |
| TRAIL-R2 | Death receptor 5, | TNFRSF10B | Apo2L) |
| | KILLER, | | |
| | TRICK2A, CD262 | | |
| TRAIL-R3 | Decoy receptor 1, | TNFRSF10C | |
| | LIT, TRID, CD263 | | |
| TRAIL-R4 | Decoy receptor 2, | TNFRSF10D | |
| | TRUNDD, CD264 | | |
| Death receptor 6 | CD358 | TNFRSF21 | |
| Death receptor 3 | Apo-3, TRAMP, | TNFRSF25 | TL1A |
| | LARD, WS-1 | | |
| FN14 | TWEAK receptor, | TNFRSF12A | TWEAK |
| | CD266 | | |

In one embodiment the anti-inflammatory agent is an agent which inhibits the activity of a Caspase.

In one embodiment the anti-inflammatory agent is an agent which inhibits the activity of Caspase-8, Caspase-10 or Caspase-8 and Caspase-10.

In one embodiment the anti-inflammatory agent is an agent which inhibits the activity of Caspase-1, Caspase-4/5 or Caspase-1 and Caspase-4/5.

In one embodiment the anti-inflammatory agent is an agent which inhibits the activity of Caspase-3, Caspase-7 or Caspase-3 and Caspase-7.

In one embodiment the anti-inflammatory agent is an agent which inhibits the activity of RIPK1 kinase or RIPK3 kinase.

In one embodiment the anti-inflammatory agent is an agent which inhibits the activity of MLKL.

In one embodiment the anti-inflammatory agent is an agent which inhibits the activity of Gasdermin D and/or Gasdermin E and/or Gasdermin C and/or Gasdermin A.

In one embodiment the anti-inflammatory agent is an agent which inhibits the activity of IL-1alpha; and/or IL-1beta; and or an agent that neutralises IL-1 receptor and/or diminishes IL-1 receptor/ligand activity.

In one embodiment the anti-inflammatory agent is an agent which inhibits the activity of IL-18; and/or an agent that neutralises IL-18 receptor and/or diminishes IL-18 receptor/ligand activity.

In one embodiment the anti-inflammatory agent is an agent which inhibits TLR3, or neutralises a ligand of TLR3, optionally via interfering with the dsRNA/TLR3 interaction

In one embodiment the anti-inflammatory agent is an NSAID, optionally selected from aspirin, inhibitors of COX-1 and/or COX-2 (e.g. celecoxib), ibuprofen, paracetamol etc.

In one embodiment the anti-inflammatory agent is a SAID, optionally selected from triamcinolone, prednisolone, 6 methyl-prednisolone, dexamethasone, cortisol and/or related corticosteroids etc. Other clinically used steroids include prednisone, Triamcinolone, Betamethasone (see e.g. Safiya Shaikh, Himanshu Verma, Nirmal Yadav, Mirinda Jauhari, Jyothi Bullangowda, “Applications of Steroid in Clinical Practice: A Review”, International Scholarly Research Notices, vol. 2012, Article D 985495, 11 pages, 2012). Other steroids are described in “Clinical Pharmacology of Corticosteroids” Dennis M Williams, Respiratory Care June 2018, 63 (6) 655-670.

In one embodiment the anti-inflammatory agent is an agent which inhibits type 1 interferon receptor, e.g. an antibody directed against the IFN-alpha receptor (IFNAR) which inhibits the binding of type 1 IFNs (i.e. IFN-alpha and IFN-beta) to this receptor and therefore interferes with type 1 IFN-mediated effects.

Methods described herein may comprise administering to a subject in need of such treatment a “therapeutically effective” amount or number of agents that decrease the biological activity of the ligand(s) or receptor(s). Agents capable of decreasing the biological activity may achieve their effect by a number of means. For instance, such an agent may be one which (by way of non-limiting example) decreases the expression of the receptor; increases receptor desensitisation or receptor breakdown; reduces interaction between ligands their endogenous receptors; reduces receptor mediated intracellular signalling; competes with endogenous receptors for ligand binding; binds to the receptors to block ligand binding; or binds to the ligand preventing interaction with its receptors.

It is preferred that the agents directly interact with the receptor or ligand.

Thus, a suitable inhibitor of a receptor/ligand interactions as discussed herein may be an agent that either:
(i) prevents or inhibits the ligand from binding to the receptor;
(ii) disrupts the receptor/ligand complex resulting from such binding.

Examples of neutralising agents suitable for use in the invention are described in more detail hereinafter. They include small molecules, antibodies or fragments thereof that bind to and neutralise the receptor or ligand, single or double-stranded nucleotide (DNA, RNA (SIRNA, miRNA, shRNA), PNA, DNA-RNA-hybrid molecule) that interfere with expression of the receptor or ligand.

JP2002114800 relates to peptides based on receptor sequences and which are reported to have inhibitory activity against TNF, TRAIL and FasL. These are said to be useful for inhibiting cell death and inflammation caused by these ligands.

In one preferred embodiment the agent binds to and blocks activity of the receptor or ligand, or it binds and blocks the endogenous ligand/receptor complex from forming properly so that it can no longer engage in the intracellular signalling.

An example of a biotherapeutic drug that can interact with such targets is an antibody, for example a human or humanised antibody. The antibodies in this invention may be monoclonal, polyclonal, chimeric, single chain antibodies (e.g. nanobodies) or functional antibody fragments.

Another example of a biotherapeutic drug is a soluble receptor protein, e.g. a soluble receptor-Fc fusion protein which contains the extracellular portion of the receptor, or at least a portion thereof that is capable of binding to the ligand in a manner that (the receptor-stimulating activity of) the respective ligand in question is inhibited.

For example, the invention may use agents that bind to RIPK1, RIPK3, MLKL, caspase-1, caspase-8, caspase-4/5 or caspase-10, for example, a small molecule or fragment thereof that binds selectively or specifically to RIPK1, RIPK3, MLKL, caspase-1, caspase-8, caspase-4/5 or caspase-10, neutralising their activity, for example by blocking their respective kinase or protease activity.

The invention may optionally employ agents each of which is a fusion protein comprising an extracellular or other domain of TNFR1, TNFR2, a TRAIL-R, preferably TRAIL-R2 or TRAIL-R1, OPG, Fas (CD95/APO-1), FLINT, DR3, FN14, TLR3, TLR4, IFNAR, or a portion thereof, fused to a portion of a human antibody, preferably an Fc domain, or a portion thereof, with or without the antibody hinge region, or a portion thereof. It should be noted that OPG and FLINT may be used as full-length proteins without the need to be fused, e.g., to an Fc portion, to block TRAIL and FasL, respectively.

Examples of agents include those that decrease the biological activity of TNFR1 and/or TNFR2, any of the TRAIL-Rs, preferably TRAIL-R1 and/or TRAIL-R2, Fas, FN14, DR3, TLR3, TLR4, or IFNAR by:
(a) decreasing the expression of the receptors;
(b) increasing receptors' desensitisation or receptors' breakdown;
(c) reducing interaction between TRAIL, FasL, TWEAK, TL1A, TNF/LT-α, dsRNA, LPS, type 1 IFNs and their respective endogenous receptors;
(d) reducing receptors' mediated intracellular signalling;
(e) competing with endogenous receptor for TRAIL, FasL, TWEAK, TL1A, TNF/LT-α, dsRNA, LPS, type 1 IFNs;
(f) binding to the receptor to block TRAIL, FasL, TWEAK, TL1A, TNF/LT-α, dsRNA, LPS, type 1 IFN binding; or
(g) binding to TRAIL, FasL, TWEAK, TL1A, TNF/LT-α, dsRNA, LPS, type 1 IFNs preventing interaction with the receptors.
(h) reducing the kinase activity of RIPK1 and/or RIPK3;
(i) reducing the protease activity of caspase-8 and/or caspase-10;
(j) reducing the expression of RIPK1, RIPK3, MLKL and/or caspase-8 and/or caspase-10;
(k) reducing the interaction of RIPK1 with RIPK3 and/or caspase-8 and/or Caspase-10;
(l) reducing the interaction of RIPK3 with MLKL;
(m) reducing the intracellular signalling of RIPK1, RIPK3, MLKL and/or caspase-8 and/or caspase-10.

Inhibitors which act on the ligands recited in the claims are available commercially or are described herein.

Preferred inhibitors are shown in Table 2.

TABLE 2
Inhibitors which may be used in the invention

| Target | Inhibitor | References |
|---|---|---|
| TNF/LT-alpha | Etanercept | (Croft and Siegel 2017) |
| TNF | Infliximab | (Croft and Siegel 2017), |
| | | U.S. Pat. No. 5,919,452 A |
| TNF | Adalimumab | (Croft and Siegel 2017), EP 0914157 B1 |
| TNF | Golimumab | (Croft and Siegel 2017) |
| TNF | Certolizumab | (Croft and Siegel 2017), WO 2013087912 A1 |
| | pegol | |
| TNF | TNF-kinoid | (Croft and Siegel 2017), WO 2007022813 A2 |
| LT-alpha | Anti LT-alpha | (Chiang et al. 2009; Kennedy et al. 2014) |
| | antibody | |
| | Pateclizumab | |
| LT- | LT-beta | (Fava et al. 2011; St Clair et al. 2018) |
| alpha1/LTbeta2 | receptor | |
| heterotrimer | fusion protein | |
| and LIGHT | Baminercept | |
| FasL (CD95L) | Asunercept | (Wick et al. 2014), EP 1447093 A1, WO |
| | (APG101) | 2004071528 A1 |
| FasL | FLINT | EP 1020521 A1 |
| Fas/FasL | antibody | WO 2010006772 A3 |
| | against FasL | |
| | or an antigen- | |
| | binding | |
| | fragment | |
| | thereof; | |
| | soluble Fas | |
| | molecule | |
| TRAIL | TRAIL-R2-Fc | WO2015001345 (Wang, Zhang, Wang, et al. 2020) |
| TLR3 | TLR3 | U.S. Pat. No. 8153583 B2 |
| | antagonist | |
| | antibody | |
| TLR3 | Peptide-GNP | (Yang et al. 2016) |
| | hybrid | |
| TLR3 | Small | (Cheng, Wang, and Yin 2011) |
| | molecules | |
| Caspases | Emricasan | (Hoglen et al. 2004) |
| Caspases | GS-9450 | (Arends et al. 2011; Ratziu et al. 2012) |
| Caspase-1 | Pralnacasan | (Rudolphi et al. 2003) |
| | (VX-740) | |
| RIPK1 | Ponatinib | (Fauster et al. 2015) |
| RIPK1 | Pazopanib | (Fauster et al. 2015) |
| RIPK3 | Kongensin A | (Li et al. 2016) |
| RIPK3 | Celastrol | (Jia et al. 2015) |
| RIPK1 | GSK2982772 | (Hamilton et al. 2019; Hou et al. 2019; Wang et al. |
| | GSK′095 | 2019; Weisel et al. 2017; Harris et al. 2017) |
| | DNL-747 | |
| | RIPK1 Ki | |
| | PK68 | |
| | Cpd-71 | |
| IFNAR | MEDI-54 | NCT01559090 |
| | (Anifrolumab) | |
| IL1-β/IL1-βR | Anakinra | (Huet et al. 2020) |
| | Canakinumab | (Ridker 2019; Zheng et al. 2019) |
| | Rilonacept | (Gomez-Garcia et al. 2018; Kapur and Bonk 2009) |
| | Gevokizumab | (Knickelbein et al. 2016; Owyang et al. 2011) |
| NLRC4/IL-18 | Tadekinig- | (Kiltz et al. 2020) |
| | alpha | |
| Canonical | MAS825 | (Mangan et al. 2018) |
| NLRP3 | | https://clinicaltrials.gov/ct2/show/record/NCT04382651 |
| Canonical and | OLT1177 | (Toldo and Abbate 2018) |
| noncanonical | | |
| NLRP3 | | |
| NLRP3 | Tranilast | (Platten et al. 2005) |
| Canonical and | MCC950 | CAS 256373-96-3 |
| noncanonical | | |
| NLRP3 | | |
| Gasdermin D | Disulfiram | (Hu et al. 2020) |

Some of these will now be described in more detail:

Blockade of TNF has been extensively used in the clinic and there are several inhibitors of TNF (signalling) available (Monaco, C., Nanchahal, J., Taylor, P. & Feldmann, M. Anti-TNF therapy: past, present and future. International immunology 27, 55-62, doi: 10.1093/intimm/dxu102 (2015)).

Commercially available monoclonal TNF-neutralising antibodies or recombinant proteins are, for example: Etanercept/Enbrel (Amgen, Pfizer) which is a TNFR2-Fc fusion protein that neutralises TNF and LT-alpha; Infliximab/Remicade from (Johnson & Johnson); Adalimumab/Humira from (AbbVie Inc.); Golimumab/Simponi (Janssen Biotech); Certolizumab/Cimzia (UCB). Given the high presence of both, LT-alpha and TNF in the lungs of SARS-CoV-MA15-infected mice (FIG. 2), Etanercept/Enbrel, which inhibits both, LT-alpha and TNF, may be preferred to other TNF-inhibitory drugs, as they inhibit TNF but not LT-alpha, in treating COVID-19 or related diseases.

Emricasan is an orally active pan-caspase protease inhibitor suitable for use against Caspases, including against caspase-8.

Inhibition of TLR3 signalling can be achieved by small molecules that act as direct, competitive and high affinity inhibitors of dsRNA binding to TLR3.

Ponatinib inhibits both RIPK1 and RIPK3, whereas pazopanib preferentially targets RIPK1 (Fauster et al CDDis 2015). Kongensin A and Celastrol are known RIPK3 inhibitors.

Agents which inhibit IL-6; and/or IL-6R; and/or IL6/sIL6R heterodimer are known in the art.

A preferred agent for the combination described herein is the IL-6/sIL-6R trans-signalling inhibitor gp130-Fc, also referred to as soluble gp130-Fc (sgp130-Fc) or olamkicept (developed by Ferring Pharmaceuticals and I-MAB Biopharma-licensed from CONARIS Research Institute-alternative names FE-301; FE-999301; TJ-301)—see e.g. Acute Inflammation: Immunity 2001; Blood 2007; Blood 2008; Sepsis: Critical Care Med 2011; J Immunol 2011; Pancreatitis-lung failure: J Clin Invest 2013; Obesity-induced inflammation: Cell Metabolism 2015; Lung Emphysema: Am J Resp Crit Care 2016; Nephrotoxic Nephritis: J Am Soc Nephrol 2015; Arteriosclerosis: Arterioscler Thromb Vasc Biol 2012; Intestinal Inflammation: Nature Medicine 2000; Gut 2006; J Immunol 2013; Rheumatoid Arthritis: J Immunol 2003; Arthritis Rheum 2006; J Immunol 2009; Asthma: J Clin Invest 2005; J Allergy Clin Immunol 2015; Bone Fracture Healing: Am J Pathol 2018; Naunyn-Schmiedeberg Arch Phamacol 2018; Lupus Erythematosus: Arthritis Rheum 2013; Tuberculosis: Immunobiology 2012; Ovarian Hyperstimul Syndrome: J Clin Endocrin Metab 2013; Listeriosis: J Immunol 2013; Abdominal Aortic Aneurism: Circ Genom Precis Med 2019; Colon cancer: Immunity 2004; Cancer Cell 2009; J Immunol 2010; J Exp, Med 2018; Ovarian cancer: Cancer Res 2011; Pancreatic cancer: Cancer Cell (2011); Int J Cancer 2015; KRAS-driven lung cancer: Cancer Res 2016; Liver cancer: Hepatology 2017; (Garbers et al. 2018).

For brevity embodiments herein may be described by way of non-limiting example with respect to TRAIL, or a TRAIL-R such as TRAIL-R2. Nevertheless, it will be appreciated that all such discussion applies mutatis mutandis to any other TRAIL-R—for example TRAIL-R1, TRAIL-R3, TRAIL-R4. It will also be appreciated that all such discussion applies mutatis mutandis to other ligands and their respective receptors described herein, including activation of inhibitory receptors e.g. Osteoprotegerin (OPG).

The invention may utilise an agent which decreases the biological activity of any, or a combination, of the TRAIL-Rs, preferably TRAIL-R1 and/or TRAIL-R2, or TRAIL by:
(a) decreasing the expression of the receptor(s);
(b) increasing receptor desensitisation or receptor breakdown;
(c) reducing interaction between TRAIL and the receptor(s) which is (are) (an) endogenous receptor(s);
(d) reducing receptor-mediated intracellular signalling;
(e) competes with endogenous receptor(s) for TRAIL binding;
(f) binds to the receptor(s) to block TRAIL binding; or
(g) binds to TRAIL preventing interaction with the receptor(s).

For agents which bind to and neutralise TRAIL, an antibody or fragment thereof that binds to and neutralises TRAIL.

Commercially available monoclonal TRAIL-neutralising antibodies are, for example anti-human TRAIL clone 2E5 from Enzo (http://www.enzolifesciences.com/ALX-804-296/trail-human-mab-2e5/) and Anti-TRAIL antibody [75411.11] (ab10516) from Abcam (http://www.abcam.com/TRAIL-antibody-75411-11-ab10516.html).

The invention may utilise an agent that binds to TRAIL-R2, e.g. an antibody, or fragment thereof, that binds specifically to TRAIL-R2, neutralising its activity.

The invention may utilise an agent that binds to TRAIL-R1, e.g. an antibody, or fragment thereof, that binds specifically to TRAIL-R1, neutralising its activity.

The invention may utilise an agent that binds to TRAIL-R1 and TRAIL-R2, e.g. an antibody, or fragment thereof, that binds specifically to TRAIL-R1 and TRAIL-R2, neutralising their activity.

By way of non-limiting example, a TRAIL inhibitor comprises the extracellular domain of TRAIL-R1, TRAIL-R2, TRAIL-R3, or TRAIL-R4 (or OPG or a ligand-binding portion thereof), preferentially that of TRAIL-R2, or a ligand-binding portion thereof, or the extracellular domain of the mature TRAIL-R2 sequence according to Walczak et al. (Walczak et al. 1997) and a patent by C. T. Rauch and H. Walczak (U.S. Pat. No. 6,569,642 B1), which is specifically incorporated herein by reference, which may be fused to a heterologous polypeptide domain, particularly an Fc portion of an immunoglobulin molecule, including or not the hinge region or part thereof, e.g. from a human IgG molecule, preferably an Fc region of human IgG1, IgG2, IgG3 or human IgG4 with or without the hinge region or a part thereof.

The way the two fully human protein parts are fused can be done in a manner that reduces the immunogenicity potential of the resulting fusion protein as described in Walczak (WO/2004/085478; PCT/EP2004/003239: “Improved Fc fusion proteins”).

Because there are two splice forms of TRAIL-R2 expressed and the splicing affects the extracellular domain of TRAIL-R2 (Screaton et al. 1997), at least two extracellular domains of TRAIL-R2 with differing amino acid sequences are known. In one embodiment, the TRAIL-binding portion of the extracellular domain of TRAIL-R2 can come from either one of these two when constructing TRAIL-inhibiting TRAIL-R2 fusion proteins.

TRAIL-R2-Fc fusions suitable for use in the present invention are described in WO2015001345, the contents of which, particularly in respect of TRAIL-R2-Fc fusions, is explicitly incorporated herein by cross reference. The TRAIL-R2-Fc polypeptide from WO2015001345 is set out below. The TRAIL-R2 portion is underlined. The Fc portion is depicted in bold. Note that there is a one amino acid overlap between the TRAIL-R2 portion and the human IgG1 Fc portion. The leader peptide is depicted in italics. The mature protein starts with the sequence ITQQDLA. When produced recombinantly, the exact position of the N terminus can vary by a few amino acids; that means the mature protein can be, e.g. one to five amino acids shorter or longer.

(SEQ ID NO: 1)
MEQRGQNAPAASGARKRHGPGPREARGARPGPRVPKTLVLVVAAVLLLV
SAESALITQQDLAPQQRAAPQQKRSSPSEGLCPPGHHISEDGRDCISCK
YGQDYSTHWNDLLFCLRCTRCDSGEVELSPCTTTRNTVCQCEEGTFREE
DSPEMCRKCRTGCPRGMVKVGDCTPWSDIECVHKESGTKHSGEVPAVEE
TVTSSPGTPASCDKTHTCPPCPAPELLGGPSVFLFPPKPKDTLMISRTP
EVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSF
FLYSKLTVDKSRWQQGNVFSCSVMHEALHNHYTQKSLSLSPGK

TRAIL-R fusion proteins that bind to and neutralise TRAIL activity may be produced using any technique that provides for the production of recombinant and non-recombinant full length or functional fragments of these proteins by continuous cell lines in culture.

Resulting proteins may be used with or without modifications such as labelling, recombinant joining of antibody stretches or with molecules functioning as reporters. Modifications can be covalent and/or non-covalent.

Variants of SEQ ID NO: 1 may be utilised in the present invention.

FasL-binding protein consisting of the extracellular domain of human Fas fused to the Fc region of human lgG1 has been used to block Fas signalling (Boch et al. 2018; Tuettenberg et al. 2012; Wick et al. 2014). FasL inhibitors include Asunercept (soluble Fas-Fc; also known as APG101; developed by Apogenix AG (Tuettenberg et al. 2012; Wick et al. 2014; Boch et al. 2018)), an anti-human FasL antibody (RNOK203) developed by Mochida Pharmaceuticals (Japan) (Poonia et al. 2009; Nisihara et al. 2001) and a genetically engineered analog of DcR3 termed FLINT (Wroblewski et al. 2003).

The methods of treatment of the present invention may be combination therapies, utilising at least 2, 3, or more agents.

For example, they may include:
(ia) a Fas ligand (FasL)-inhibiting agent that neutralises FasL and/or Fas, i.e. the cellular receptor of FasL, and/or diminishes the interaction of FasL with Fas and/or the activity resulting from this interaction, and/or
(ib) a TRAIL-inhibiting agent that neutralises TRAIL and/or a TRAIL-R which is optionally TRAIL-R2 and/or diminishes the interaction of TRAIL with one or more than one of its receptors, and/or the activity resulting from this interaction, and
(ii) an anti-inflammatory agent.

The agents may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4, or 12 or more hours apart, or even longer periods, e.g. 1, 2, 3, 4, 7, 14, or 21 days, apart where required), the precise dosage regimen being commensurate with the properties of the therapeutic agent(s).

The agents (i.e., a compound as described here, plus one or more other agents) may be formulated together in a single dosage form, or alternatively, the individual agents may be formulated separately and presented together in the form of a kit, optionally with instructions for their use.

In another embodiment the combinatorial therapies in this invention may be administered in combination with at least one other therapeutically active agent, wherein the other therapeutically active agent appropriate to the condition being treated e.g. ARDS or other disease caused by an airway-targeting virus such as a coronavirus or an influenza virus.

Other COVID-19 combination treatments include one or more of: lopinavir-ritonavir; arbidol; azithromycin, remdesivir, favipiravir, anti-inflammatory treatments such as actemra (tocilizumab), corticosteroids such as, e.g. dexamethasone, recently identified SARS-CoV-2-neutralising antibodies (see e.g. (Kreer et al. 2020)) and other treatments such as convalescent plasma (see e.g. (Thorlund et al. 2020)).

In some embodiments the agent or agents may be administered to a subject or individual during late-stage disease.

A treatment regimen based on the agent or agents described herein will preferably extend over a sustained period of time appropriate to the disease and symptoms. The particular duration would be at the discretion of the physician.

For example, the duration of treatment may be:
1 to 14, e.g. 1, 2, 3, 4, 5, 6, 7, 8, 9, 10, 11, 12, 13 or 14 days.
1 to 4, e.g. 1, 2, 3 or 4 weeks.

For prophylaxis, the treatment may be ongoing.

In all cases the treatment duration will generally be subject to advice and review of the physician.

In some embodiments the subject may be a human who has been diagnosed as having (“confirmed”) COVID-19, or wherein said method comprises making said diagnosis.

Diagnosis of COVID-19 may be via any method known in the art. Examples include laboratory testing for the presence of the SARS-CoV-2 virus—for example directly based on the presence of virus itself (e.g. using RT-PCR and isothermal nucleic acid amplification, or the presence of antigenic proteins) or indirectly via antibodies produced in response to infection. Other methods of diagnosis include chest X-ray, optionally in combination with characteristic symptoms as described below (see e.g. Li, Xiaowei, et al. “Molecular immune pathogenesis and diagnosis of COVID-19.” Journal of Pharmaceutical Analysis (2020); Fang, Yicheng, et al. “Sensitivity of chest CT for COVID-19: comparison to RT-PCR.” Radiology (2020): 200432; Chan, Jasper Fuk-Woo, et al. “Improved Molecular Diagnosis of COVID-19 by the Novel, Highly Sensitive and Specific COVID-19-RdRp/Hel Real-Time Reverse Transcription-PCR Assay Validated In Vitro and with Clinical Specimens.” Journal of Clinical Microbiology 58.5 (2020); Tang, Yi-Wei, et al. “The laboratory diagnosis of COVID-19 infection: current issues and challenges.” Journal of Clinical Microbiology (2020)).

In some embodiment the subject is a human who has been assessed as being “at risk” of, COVID-19, or having probable COVID-19, e.g. based on situational or other data.

Those who are at particular risk of COVID-19 include:
People who have been in close contact with one or more COVID-19 cases
People 65 years and older;
People who live in a nursing home, care home, or long-term care facility;
People of all ages with relevant underlying medical conditions, particularly if not well controlled, including:
   People with chronic lung disease or moderate to severe asthma
   People who have serious heart conditions
   People who are immunocompromised
      As is known in the art, many conditions can cause a person to be immunocompromised, including cancer treatment, smoking, bone marrow or organ transplantation, immune deficiencies, poorly controlled HIV or AIDS, and prolonged use of corticosteroids and other immune weakening medications
   People with severe obesity (body mass index [BMI] of 40 or higher)
   People with diabetes
   People with chronic kidney disease undergoing dialysis
   People with liver disease

Symptoms or circumstances which are indicative of potential (“probable”) COVID-19 include:
1) a patient with acute respiratory tract infection (sudden onset of at least one of the following: cough, fever, shortness of breath) AND with no other aetiology that fully explains the clinical presentation AND with a history of travel or residence in a country/area reporting local or community transmission during the 14 days prior to symptom onset;
OR
2) a patient with any acute respiratory illness AND having been in close contact with a confirmed or probable COVID-19 case in the last 14 days prior to onset of symptoms;
OR
3) A patient with severe acute respiratory infection (SARI) (fever and at least one sign/symptom of respiratory disease, e.g., cough, fever, shortness breath) AND requiring hospitalisation AND with no other aetiology that fully explains the clinical presentation. “Close contact” as used herein is defined as:
   A person living in the same household as a COVID-19 case;
   A person having had direct physical contact with a COVID-19 case (e.g. shaking hands);
   A person having unprotected direct contact with infectious secretions of a COVID-19 case (e.g. being coughed on, touching used paper tissues with a bare hand);
   A person having had face-to-face contact with a COVID-19 case within 2 metres and >15 minutes;
   A person who was in a closed environment (e.g. classroom, meeting room, hospital waiting room, etc.) with a COVID-19 case for 15 minutes or more and at a distance of less than 2 metres;
   A healthcare worker (HCW) or other person providing direct care for a COVID-19 case, or laboratory workers handling specimens from a COVID-19 case without recommended personal protective equipment (PPE) or with a possible breach of PPE;
   A contact in an aircraft sitting within two seats (in any direction) of the COVID-19 case, travel companions or persons providing care, and crew members serving in the section of the aircraft where the index case was seated (if severity of symptoms or movement of the case indicate more extensive exposure, passengers seated in the entire section or all passengers on the aircraft may be considered close contacts).

The epidemiological link to a probable or confirmed case may have occurred within a 14-day period before the onset of illness in the suspected case under consideration. In some embodiments the individual or subject may suffer a co-morbidity such as those described above e.g. diabetes or obesity, and/or a secondary bacterial infection and/or suffers lymphopenia.

The specification herein defines methods of treating diseases using specified agents or combinations of agents.

Further disclosed herein are corresponding agents or combinations of agents for use in the treatment of those diseases.

For example, there is disclosed a TRAIL-inhibiting agent that neutralises TRAIL and/or a TRAIL-R which is optionally TRAIL-R2 and/or diminishes TRAIL/TRAIL-R activity and/or a FasL-inhibiting agent that neutralises FasL and/or a receptor thereof and/or diminishes Fas/FasL activity, for use in the treatment of diseases described herein.

For example, there is disclosed a TRAIL-inhibiting agent that neutralises TRAIL and/or a TRAIL-R which is optionally TRAIL-R2 and/or diminishes TRAIL/TRAIL-R activity and/or a FasL-inhibiting agent that neutralises FasL and/or a receptor thereof and/or diminishes Fas/FasL activity, for use in the treatment of diseases described herein, wherein the treatment comprises use of an anti-inflammatory agent.

For example, there is disclosed an anti-inflammatory agent for use in the treatment of diseases described herein, wherein the treatment comprises use of a TRAIL-inhibiting agent that neutralises TRAIL and/or a TRAIL-R which is optionally TRAIL-R2 and/or diminishes TRAIL/TRAIL-R activity and/or a FasL-inhibiting agent that neutralises FasL and/or a receptor thereof and/or diminishes FasL/Fas activity.

Likewise, further disclosed herein are use of the corresponding agents or combinations of agents in the preparation of a medicament for use in the treatment of those diseases.

To summarise some of the foregoing, the reported upregulation of TRAIL, FasL, LT-alpha and TNF in the lungs of infected mice support a rationale whereby the activation of their respective receptors leads to cell death-induced inflammation (CDI) (as shown with the Examples provided above). However, the activation of these receptors by their respective ligands also leads to the transcriptional activation of genes which, independently of the cell death induced by these receptor-ligand interactions, will contribute to inflammation and to the recruitment and infiltration of immune molecules which will, in turn, shape the cellular microenvironment to accommodate the exacerbation of inflammatory stimulation and the rewiring of inflammation-induced cell death (ICD). Importantly, for TRAIL it has been shown that the stimulation of epithelial cells which are not killed by TRAIL leads to the production of various cytokines, including CCL2 (Hartwig et al. 2017). This TRAIL-induced CCL2 production was shown to be responsible for triggering an immune microenvironment which prevented immune recognition of lung cancer and also promoted lung cancer growth through the recruitment of CCR2-expressing myeloid cells into the tumor immune microenvironment (Hartwig et al. 2017). Intriguingly, CCL2 has been implicated in the recruitment of harmful immune cells to the lungs of patients suffering from COVID-19 (Blanco-Melo et al. 2020). Thus, the inhibition of TRAIL, but equally that of other death ligands may therefore owe its therapeutic efficacy not only to the inhibition of cell death but also to the inhibition of an immune microenvironment which prevents or at least hinders immune recognition and, thereby, interferes with the development of an effective and timely adaptive immune response to SARS-CoV-2.

These results are consistent with the results we previously published (Peltzer et al. 2018; Taraborrelli et al. 2018) that concomitant inhibition of various death receptor-ligand systems is able to afford a therapeutic response in an inflammatory disease. This, together with the aforementioned in-vivo results showing that combined inhibition of cell death and inflammation, but not of either cell death or inflammation alone, is of therapeutic value, leads us to propose and support the validity of the therapeutic concept of the inhibition of death ligands, both individually and in combination, in addition to anti-inflammatory drugs for the treatment of a viral disease which induces type 1 and/or type 2 interferons. This includes, but is not limited to, infection by an influenza or a coronavirus, including the coronavirus responsible for COVID-19, SARS-CoV-2.

Consequently, the end stage of COVID-19 can be envisaged as that of an unfair battle field in which cells that are sensitised to death ligand-induced cell death are faced with the overwhelming presence of death ligands, likely mainly expressed on various types of infiltrating immune cells but potentially also by neighbouring epithelial and/or endothelial cells, or by fibroblasts in the lungs of affected individuals.

Importantly, this or a similar mechanism is likely to apply to ARDS induced by other viruses and/or non-viral infection as well as to ARDS in general and may also extend to sepsis and other diseases, including multi-organ failure (as exemplified by the type 1 and type 2 IFN-mediated sensitisation of non-transformed epithelial cells obtained from various human tissues to death ligand-induced cell death), whether induced as a consequence of ARDS or independently thereof.

### Antibodies

For the production of antibodies according to the invention, various host species may be immunised by injection with the above-mentioned proteins to be targeted or any fragments of the two proteins which are immunogenic.

For example, antibodies to neutralise TRAIL activity may be raised against full length human TRAIL, sequences.

An appropriate adjuvant will be chosen depending on the host species in order to increase an immune response. Preferentially, peptides, fragments or oligopeptides used to induce an antibody response against them will contain at least five, but preferably ten amino acids. Monoclonal antibodies against the two proteins may be produced using any technique that provides for the production of antibody molecules or recombinant and non-recombinant functional fragments of these antibodies by continuous cell lines in culture. These include, but are not limited to, the hybridoma technique and the human B-cell hybridoma technique. In addition, techniques developed for the production of chimeric antibodies, e.g. recombinant antibodies, can be used. Resulting antibodies may be used with or without modifications such as labelling, recombinant joining of antibody stretches or with molecules functioning as reporters. Modifications can be covalent and/or non-covalent.

Many different immuno- and non-immuno-assays may be used for screening to identify antibodies with the desired specificity. Various protocols for competitive binding and immuno-radiometric assays using either polyclonal or monoclonal antibodies with already established specificity are well known in the field. These immunoassays typically involve measuring complex formation between the receptor or ligand and their specific antibodies. A “Sandwich”, i.e. two-sided, monoclonal-based immunoassay is preferred that comprises monoclonal antibodies against two non-interfering protein epitopes, but a competitive binding assay may also be used.

More specifically, it is preferred that the antibody is a γ-immunoglobulin (IgG). It will be appreciated that the variable region of an antibody defines the specificity of the antibody and, as such, this region should be conserved in functional derivatives of the antibody according to the invention. The regions beyond the variable domains (C-domains) are relatively constant in sequence. It will be appreciated that the characterising feature of antibodies according to the invention is the V_{H} and V_{L} domains. It will be further appreciated that the precise nature of the C_{H} and C_{L} domains is not, on the whole, critical to the invention. In fact, preferred antibodies according to the invention may have very different C_{H} and C_{L} domains. Furthermore, preferred antibody functional derivatives may comprise the Variable domains without a C-domain (e.g. scFV antibodies). Other preferred derivatives are single-domain antibodies (sdAbs), also known as nanobodies. Bi-specific antibodies are also well known in the art, and may be used in the present invention.

An antibody derivative may have 75% sequence identity, more preferably 90% sequence identity and most preferably it has at least 95% sequence identity to a monoclonal antibody or specific antibody in a polyclonal mix. It will be appreciated that most sequence variation may occur in the framework regions (FRs) whereas the sequence of the CDRs of the antibodies, and functional derivatives thereof, is most conserved.

A number of preferred embodiments of the invention relate to molecules with both Variable and Constant domains. However, it will be appreciated that antibody fragments (e.g. scFV antibodies) are also encompassed by the invention that comprise essentially the Variable region of an antibody without any Constant region.

Antibodies generated in one species are known to have several serious drawbacks when used to treat a different species. For instance, when murine antibodies are used in humans they tend to have a short circulating half-life in serum and are recognised as foreign proteins by the patient being treated. This leads to the development of an unwanted human anti-mouse (or rat) antibody response. This is particularly troublesome when frequent administrations of the antibody are required as it can enhance the clearance thereof, block its therapeutic effect, and induce hypersensitivity reactions. Accordingly, preferred antibodies (if of non-human source) for use in human therapy are humanised.

Monoclonal antibodies are generated by the hybridoma technique which usually involves the generation of non-human mAbs. The technique enables rodent monoclonal antibodies with almost any specificity to be produced. Accordingly, preferred embodiments of the invention may use such a technique to develop monoclonal antibodies against TRAIL or the TRAIL receptors. Although such antibodies are useful therapeutically, it will be appreciated that such antibodies are not ideal therapeutic agents in humans (as suggested above). Ideally, human monoclonal antibodies would be the preferred choice for therapeutic applications. However, the generation of human mAbs using conventional cell fusion techniques has not been very successful to date. The problem of humanisation may be at least partly addressed by engineering antibodies that use V region sequences from non-human (e.g. rodent) mAbs and C region (and ideally FRs from V region) sequences from human antibodies. The resulting ‘engineered’ mAbs are less immunogenic in humans than the rodent mAbs from which they were derived and are therefore better suited for clinical use.

Humanised antibodies may be chimaeric monoclonal antibodies, in which, using recombinant DNA technology, rodent immunoglobulin constant regions are replaced by the constant regions of human antibodies. The chimaeric H chain and L chain genes may then be cloned into expression vectors containing suitable regulatory elements and induced into mammalian cells in order to produce fully glycosylated antibodies. By choosing an appropriate human H chain C region gene for this process, the biological activity of the antibody may be pre-determined. Such chimaeric antibodies are superior to non-human monoclonal antibodies in that their ability to activate effector functions can be tailored for a specific therapeutic application, and the anti-globulin response they induce is reduced.

Such chimaeric molecules are preferred agents for treating disease according to the present invention. RT-PCR may be used to isolate the V_{H} and V_{L} genes from preferred mAbs, cloned and used to construct a chimaeric version of the mAb possessing human domains.

Further humanisation of antibodies may involve CDR-grafting or reshaping of antibodies. Such antibodies are produced by transplanting the heavy and light chain CDRs of a rodent mAb (which form the antibody's antigen binding site) into the corresponding framework regions of a human antibody.

### Peptide Agents

It will be appreciated that peptide or protein agents used or provided according to the invention may be derivatives of native or original sequences, and thus include derivatives that increase the effectiveness or half-life of the agent in vivo. Examples of derivatives capable of increasing the half-life of polypeptides according to the invention include peptoid derivatives, D-amino acid derivatives and peptide-peptoid hybrids.

Proteins and peptide agents according to the present invention may be subject to degradation by a number of means (such as protease activity at a target site). Such degradation may limit their bioavailability and hence therapeutic utility. There are a number of well-established techniques by which peptide derivatives that have enhanced stability in biological contexts can be designed and produced. Such peptide derivatives may have improved bioavailability as a result of increased resistance to protease-mediated degradation. Preferably, a derivative suitable for use according to the invention is more protease-resistant than the protein or peptide from which it is derived. Protease-resistance of a peptide derivative and the protein or peptide from which it is derived may be evaluated by means of well-known protein degradation assays. The relative values of protease resistance for the peptide derivative and peptide may then be compared.

Peptoid derivatives of proteins and peptides according to the invention may be readily designed from knowledge of the structure of the receptor according to the first aspect of the invention or an agent according to the fourth, fifth or sixth aspect of the invention. Commercially available software may be used to develop peptoid derivatives according to well-established protocols.

Retropeptoids, (in which all amino acids are replaced by peptoid residues in reversed order) are also able to mimic proteins or peptides according to the invention. A retropeptoid is expected to bind in the opposite direction in the ligand-binding groove, as compared to a peptide or peptoid-peptide hybrid containing one peptoid residue. As a result, the side chains of the peptoid residues are able to point in the same direction as the side chains in the original peptide.

A further embodiment of a modified form of peptides or proteins according to the invention comprises D-amino acid forms. In this case, the order of the amino acid residues is reversed. The preparation of peptides using D-amino acids rather than L-amino acids greatly decreases any unwanted breakdown of such derivative by normal metabolic processes, decreasing the amounts of the derivative which needs to be administered, along with the frequency of its administration.

### Nucleic Acids

In a further embodiment of the present invention the agent or inhibitor is a nucleic acid effector molecule.

The nucleic acid effector molecule may be DNA, RNA (including siRNA, miRNA and shRNA), PNA or a DNA-RNA-hybrid molecule. These may be specifically directed towards down-regulation of TRAIL or TRAIL-R sequences (see e.g. Example 5). siRNA forms part of a gene silencing mechanism, known as RNA interference (RNAi) which results in the sequence-specific destruction of mRNAs and enables a targeted knockout of gene expression. siRNA used in gene silencing may comprise double stranded RNA of 21 nucleotides length, typically with a 2-nucleotide overhang at each 3′ end. Alternatively, short hairpin RNAs (shRNAs) using sense and antisense sequences connected by a hairpin loop may be used. Both siRNAs and shRNAs can be either chemically synthesized and introduced into cells for transient RNAi or expressed endogenously from a promoter for long-term inhibition of gene expression. siRNA molecules for use as an agent according to the invention may comprise either double stranded RNA of 10-50 nucleotides. Preferably, siRNAs for use as an agent according to the invention comprise 18-30 nucleotides. More preferably, siRNAs for use as an agent according to the invention comprise 21-25 nucleotides. And most preferably, siRNAs for use as an agent according to the invention comprise 21 nucleotides. It will be appreciated that siRNAs will need to be based upon the sequences according to the second aspect of the invention. Preferred double stranded siRNA molecules comprise a sense strand of 21-25 contiguous nucleotides from a sequence of the TRAIL or its receptors bound to the complementary antisense strand. Alternatively, shRNAs using sense and antisense sequences may be used as an agent according to the invention. Preferably, shRNAs using sense and antisense sequences that may be employed as an agent according to the invention comprise 20-100 nucleotides.

In other embodiments the nucleic acid may encode other agents of the invention—for example the fusion proteins described.

The nucleic acid may be single or double-stranded. The nucleic acid effector molecule may be delivered directly as a drug (this could be “naked” or e.g. in liposomes) it may be expressed from a retrovirus, adenovirus, herpes or vaccinia virus or bacterial plasmids for delivery of nucleotide sequences to the targeted organ, tissue or cell population.

These constructs may be used to introduce untranslatable sense or antisense sequences into a cell.

Without integration into the DNA, these vectors may continue to produce RNA molecules until degradation by cellular nucleases. Vector systems may result in transient expression for one month or more with a non-replicating vector and longer if appropriate replication elements are part of the vector system.

Thus, as is well known in the art, recombinant vectors may include other functional elements. For instance, recombinant vectors can be designed such that the vector will autonomously replicate in the cell. In this case, elements which induce DNA replication may be required in the recombinant vector. Alternatively, the recombinant vector may be designed such that the vector and nucleic acid molecule integrates into the genome of a cell. In this case DNA sequences which favour targeted integration (e.g. by homologous recombination) are desirable. Recombinant vectors may also have DNA coding for genes that may be used as selectable markers in the cloning process. The recombinant vector may also further comprise a promoter or regulator to control expression of the nucleic acid as required.

### Variants

Wherever amino acid and nucleic acid sequences are discussed herein (for example in respect of coding fusion proteins or other agents), it will be appreciated by the skilled technician that functional derivatives of the amino acid, and nucleic acid sequences, disclosed herein, are also envisaged-such derivatives may have a sequence which has at least 30%, preferably 40%, more preferably 50%, and even more preferably, 60% sequence identity with the amino acid/polypeptide/nucleic acid sequences of any of the sequences referred to herein. An amino acid/polypeptide/nucleic acid sequence with a greater identity than preferably 65%, more preferably 75%, even more preferably 85%, and even more preferably 90% to any of the sequences referred to is also envisaged.

Preferably, the amino acid/polypeptide/nucleic acid sequence has 92% identity, even more preferably 95% identity, even more preferably 97% identity, even more preferably 98% identity and, most preferably, 99% identity with any of the referred to sequences.

For example, such variants of SEQ ID NO 1 are encompassed, provided they retain the biological activity of binding TRAIL-R.

Calculation of percentage identities between different amino acid/polypeptide/nucleic acid sequences may be carried out as follows. A multiple alignment is first generated by the ClustalX program (pair wise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

Alternatively, a substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention.

Due to the degeneracy of the genetic code, it is clear that any nucleic acid sequence could be varied or changed without substantially affecting the sequence of the receptor protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid.

The accurate alignment of protein or DNA has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA.

Frequently, automatically generated alignments require manual alignment, exploiting the trained user's knowledge of the protein family being studied, e.g., biological knowledge of key conserved sites. One such alignment editor programs is Align (http://www.gwdg.de/˜dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYLIP.

Other modifications in protein sequences are also envisaged and within the scope of the claimed invention, i.e. those which occur during or after translation, e.g. by acetylation, amidation, carboxylation, phosphorylation, proteolytic cleavage or linkage to a ligand.

### Compositions, Dosages and Regimens

Where novel combinations of agents are described herein, the combinations per se form aspects of the present invention (independent of use).

The agents utilised in the present invention may be provided as a “pharmaceutical composition” (e.g., formulation, preparation, medicament) comprising one or more agents described herein, and a pharmaceutically acceptable carrier, diluent, or excipient.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In some embodiments, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

The pharmaceutical compositions detailed in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual or rectal means.

Pharmaceutical compositions will generally comprise the agents in an effective amount to achieve the intended purpose.

The determination of an effective dose is well within the capability trained personnel. For any compounds, the therapeutically effective dose can be estimated initially either in cell culture assays, e.g., of cell lines or in animal models, usually but not exclusively mice. The animal model may also be used to determine the appropriate concentration range and route of administration. Based on such pilot experiments, useful doses and routes for administration in humans can be determined. A therapeutically effective dose refers to that amount of active ingredient, for example a nucleic acid or a protein of the invention or an antibody, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as LD50/ED50. Pharmaceutical compositions, which exhibit large therapeutic indices, are preferred. The dosage is preferably within a range of circulating concentrations that include the ED50 with little or no toxicity. The dosage varies within this range depending upon the dosage employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells and conditions as detailed above.

### General Statements

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress (prolonged survival), a halt in the rate of progress, regression of the condition, amelioration of the condition, and cure of the condition.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen.

The invention also embraces treatment as a prophylactic measure is also included and “treating” will be understood accordingly. Prophylactic treatment may utilise a “prophylactically effective amount,” which, where used herein, pertains to that amount of an agent which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

Wherever a method of treatment employing an agent is described herein, it will be appreciated that an agent (any one of the first, second, third agents) for use in that method is also described, as is an agent (any one of the first, second, third agents) for use in the manufacture of a medicament for treating the relevant inflammatory disease. Also described is any one of the first, second, third agents for use in methods of enhancing the activity of the other two agents.

Wherever a composition is described herein, it will be appreciated that the same composition for use in the therapeutic methods (including prophylactic methods) described herein is also envisaged, as is the composition for use in the manufacture of a medicament for treating the relevant inflammatory disease.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

### FIGURES

FIG. 1: A. Mice deficient in TNFR1 constitutively and lacking HOIL-1 specifically in the epidermis develop, TNFR1-independent severe skin inflammation around 30 days (left image) and have to be culled at around 70 days. These mice treated from 30 days onwards with a combination of RIPK1 kinase inhibitor (GSK457A) plus the corticosteroid Triamcinolone (TM) are protected from disease progression and skin lesions are almost resolved. B. Quantification of lesions in mice in A, including controls. Character of lesion is evaluated with increasing scored based on punctuated lesions, erosion, ulcers. Lesions first appear in the neck and subsequently expand to the back and flanks of the mouse. Regions affected is the sum of the areas in the body where lesions were found. Bars represent mean plus/minus SEM of n=6 or 7 mice. Statistics were performed by 2-way ANOVA of stacked graphs using GraphPad Prism v8.4.

FIG. 2: Correlation between the expression of individual genes of interest (X axis) and the computationally estimated cell subpopulation abundance (Y axis) in bulk RNA-seq data from C57BL/6 mice infected with 10\hat{ }2, 10\hat{ }3, 10\hat{ }4 or 10\hat{ }5 plaque-forming units (PFU) of SARS COV MA15, measured on day 1, 2, 4 and 7 post-infection (dataset GSE33266 deposited at the Gene Expression Omnibus). Cell subpopulations are coloured according to class and their abundance is estimated using Gene Set Variation Analysis. Individual datapoints correspond to different RNA-seq samples. The Pearson correlation and p-value are displayed in each subplot. Significant correlations suggest a dependency between the expression of the respective gene and the abundance of the respective cell type.

FIG. 3: NIH3T3 cells were treated with the indicated combinations of IFNs and 1 μg/ml of Death Ligand in the presence of the pan caspase inhibitor zVAD-fmk. Cells were incubated with IFNs for 24 hr and subsequently treated with IFNs and Death Ligands as indicated. Cell death was assessed via Sytox green incorporation by the Incucyte cell system. Cell death is expressed as total green object-integrated intensity as calculated via the adherent cell to cell analysis software provided by the Incucyte system.

FIG. 4: Human pancreatic ductal epithelial cells (HPDECs) were incubated with the indicated IFNs overnight before being treated with 100 ng/ml of the indicated Death Ligand, either alone or in combination with the pan caspase inhibitor zVAD or with zVAD and RIPK1 kinase inhibitor Nec-1s. Cell death was assessed via Sytox green incorporation by the Incucyte cell system. Cell death is expressed as total green object-integrated intensity as calculated via the adherent cell to cell analysis software provided by the Incucyte system.

FIG. 5: Primary human lung fibroblasts (MRC5 cells) were incubated with the indicated IFNs overnight before being treated with 1 μg/ml of the indicated Death Ligand, either alone or in combination with the pan-caspase inhibitor zVAD or with zVAD and the RIPK1 kinase inhibitor Nec-1s. Cell death was assessed via Sytox green incorporation by the Incucyte cell system. Cell death is expressed as total green object integrated intensity as calculated via the adherent cell to cell analysis software provided by the Incucyte system.

FIG. 6: MRC5 cells were treated with the indicated IFNs for 24 h before cells were lysed and cell lysates were analysed for presence of the indicated proteins by western blotting.

FIG. 7: Freshly isolated Lung endothelial cells (LECs) were incubated with the indicated IFNs overnight before being treated with 1 μg/ml of the indicated Death Ligands either alone or in combination with the pan caspase inhibitor zVAD or with zVAD and the RIPK1 kinase inhibitor Nec-1s. Cell death was assessed via Sytox green incorporation by the Incucyte cell system. Cell death is expressed as total green object-integrated intensity as calculated via the adherent cell-to-cell analysis software provided by the Incucyte system.

FIG. 8: Freshly isolated LECs were treated with the indicated IFNs for 24 h before cells were lysed and cell lysates were analysed for presence of the indicated proteins by western blotting.

FIG. 9: MRC5 cells were incubated overnight with the indicated IFNs before being treated with 1 μg/ml of the indicated Death Ligands in combination with the pan-caspase inhibitor zVAD or zVAD with BV6 (SMAC mimetic) and/or the TRAIL-blocking recombinant TRAIL-R2-Fc protein. Cell death was assessed via Sytox green incorporation by the Incucyte cell system. Cell death is expressed as total green object-integrated intensity as calculated via the adherent cell-to-cell analysis software provided by the Incucyte system.

FIG. 10: Kaplan-Meier graphs showing the survival curves of female BALB/c mice (n=5 for each group) infected with 2×10\hat{ }5 TCID50 of SARS-CoV MA15 on day 0, and treated on day 2 post-infection with the indicated combinations of soluble blockers of murine TNF, murine FasL or with the small molecule MCC950, an inhibitor of the NALP3 inflammasome. In this embodiment, the indicated recombinant soluble fusion proteins (mTNFR2-Fc and mFas-Fc) comprised the extracellular domains of the naturally expressed murine (Mus musculus-derived) proteins, TNFR2 and Fas, respectively, fused to the Fc-portion of murine IgG2a. A recombinantly produced non-binding monoclonal antibody of the mouse IgG2a isotype served as control protein. All recombinant proteins were administered intraperitoneally once at a concentration of 0.5 mg/mouse diluted in PBS. All mice received daily intraperitoneal injections of PBS+5% Glucose from day 1 to day 5 post infection, except for the days on which they were treated with the specified recombinant proteins. MCC950 was administered intraperitoneally daily from day 2 to day 5 at a dose of 0.1 mg/mouse diluted in 5% Glucose in PBS.

FIG. 11: Kaplan-Meier graphs showing the survival curves of female BALB/c mice (n=5 for each group) infected with 2×10\hat{ }5 TCID50 of SARS-CoV MA15 on day 0, and treated on day 1 with soluble murine TNF-blocking agent either alone or in combination with the anti-inflammatory agent Dexamethasone (Dexa) at two different concentrations (1 μg/mouse or 3 μg/mouse). In this embodiment, the indicated soluble murine TNF blocking agent (namely mTNFR2-Fc) was comprised of the extracellular domain of murine TNFR2 fused to the Fc-portion of murine IgG2a. A recombinantly produced non-binding monoclonal antibody of the mouse IgG2a isotype served as control protein. All mice received daily intraperitoneal injections of PBS+5% Glucose from day 1 to day 7 post infection, except for the days on which they were treated with the specified recombinant proteins. All Fc-fusion proteins were administered intraperitoneally once at a concentration of 0.5 mg/mouse diluted in PBS. Dexamethasone was administered intraperitoneally daily from day 3 to day 7 at the indicated dose diluted in PBS+5% Glucose.

FIG. 12: Kaplan-Meier graphs showing the survival curves of K18 hACE2 transgenic C57BL/6 mice (n=5 for each group) infected with 4×10\hat{ }4 TCID50 of SARS-CoV-2 on day 0, and treated on day 3 post-infection with the indicated combinations of soluble blockers of murine TNF and murine FasL. In this embodiment, the indicated recombinant soluble fusion proteins (mTNFR2-Fc and mFas-Fc) comprised the extracellular domains of the naturally expressed murine (Mus musculus-derived) proteins, TNFR2 and Fas, respectively, fused to the Fc-portion of murine IgG2a. A recombinantly produced non-binding monoclonal antibody of the mouse IgG2a isotype served as control protein. All recombinant proteins were administered intraperitoneally once at a concentration of 0.5 mg/mouse diluted in PBS. All mice received daily intraperitoneal injections of PBS+5% Glucose from day 1 to day 7 post infection, except for the days on which they were treated with the recombinant proteins.

### EXAMPLES

### Example 1: Inhibition of Cell Death and Inflammation Resolved Chronic Dermatitis

LUBAC, an E3 ligase that regulates cell death and inflammation downstream of various immune receptors, contains three components, HOIL-1, HOIP and SHARPIN. Loss of HOIL-1 in the skin results in aberrant cell death which causes severe chronic inflammation which is only partially mediated by TNF. In this cell death-driven lethal inflammatory model (Hoil-1^{E-KO} mice), prophylactic inhibition of cell death by genetic ablation of caspase-8 and RIPK3 was sufficient to prevent chronic inflammatory skin disease (Taraborrelli et al. 2018).

Likewise, prophylactic pharmacological inhibition of RIPK1 kinase activity in combination with loss of TNFR1 (Tnfr1^{KO}Hoil-1^{E-KO} mice) in pregnant females prevented onset of dermatitis in newborn mutant mice (Taraborrelli et al. 2018).

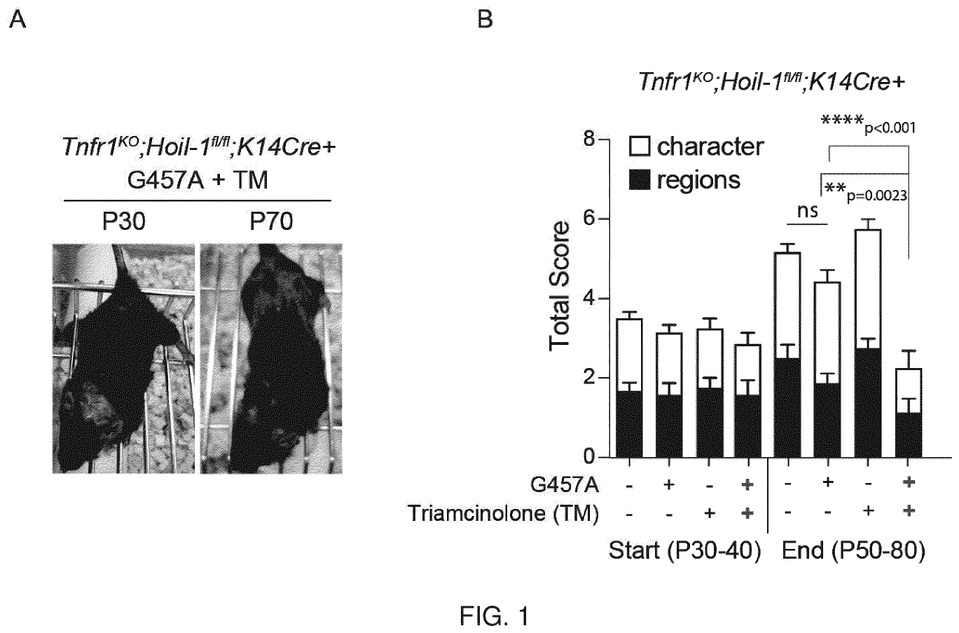

Crucially, however, we made the unexpected observation that treatment with the same RIPK1 kinase inhibitor that completely prevented onset of dermatitis in newborn Tnfr1^{KO}Hoil-1^{E-KO} mice following treatment of their pregnant mothers, i.e. when the RIPK1 inhibitor was given prophylactically, neither ameliorated nor cured the inflammation in Tnfr1^{KO}Hoil-1^{E-KO} mice, when it was applied at later times, i.e. once inflammation has been initiated. The latter scenario more closely resembles the situation that is usually encountered in the clinic, i.e. a patient presenting with an ongoing inflammatory disease rather than before the onset of inflammation. Strikingly, however, when we combined the inhibition of RIPK1 with corticosteroid treatment, which alone was also insufficient to achieve a meaningful therapeutic effect, we were able to cure the mice of the otherwise lethal inflammation (FIG. 1).

Thereby, we discover the new therapeutic principle according to which in a situation in which cell death drives inflammation and/or inflammation drives tissue damage and/or cell death and neither cell death-inhibitory nor anti-inflammatory therapeutics alone are sufficient to resolve or significantly or sustainably ameliorate an ongoing inflammation or prevent it from continuing and/or exacerbating, it is possible to provide significant therapeutic benefit through concomitant inhibition of cell death and inflammation as demonstrated in FIG. 1.

Notably, cell death and inflammation in Tnfr1^{KO};Hoil-1^{E-KO} mice was prevented by genetic loss of both Trail-r and the death domain of CD95 (Fas) but not by loss of either one alone (Taraborrelli et al. 2018). This supports the improved therapeutic potential of combinatorial therapies in which more than one death receptor/ligand system is inhibited or neutralised.

### Example 2: Global RNA-Seq Expression Data of the Lungs of Mice Infected with SARS-CoV-MA15

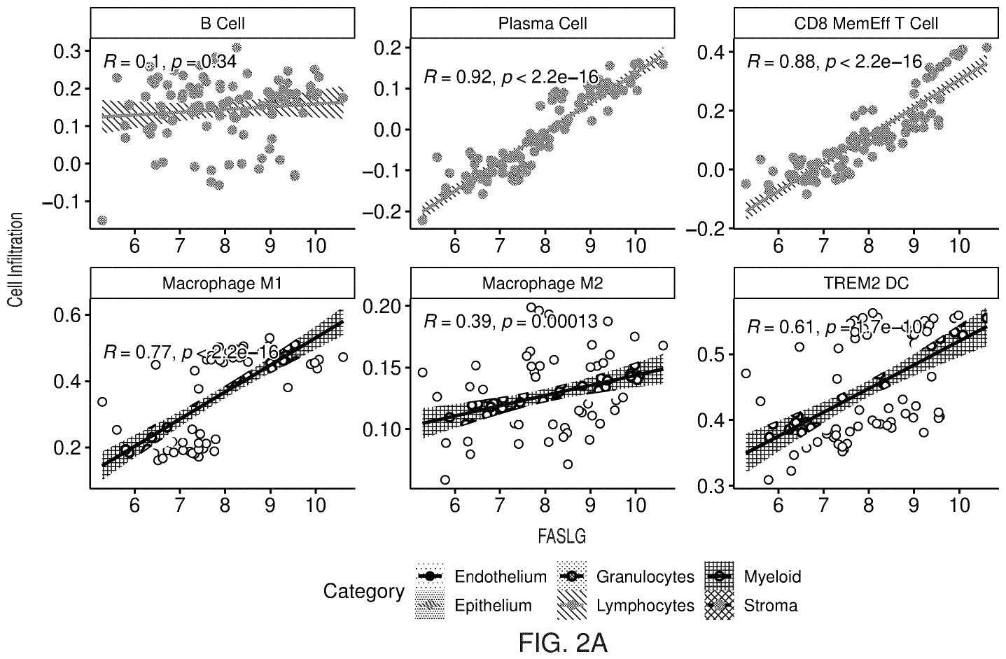

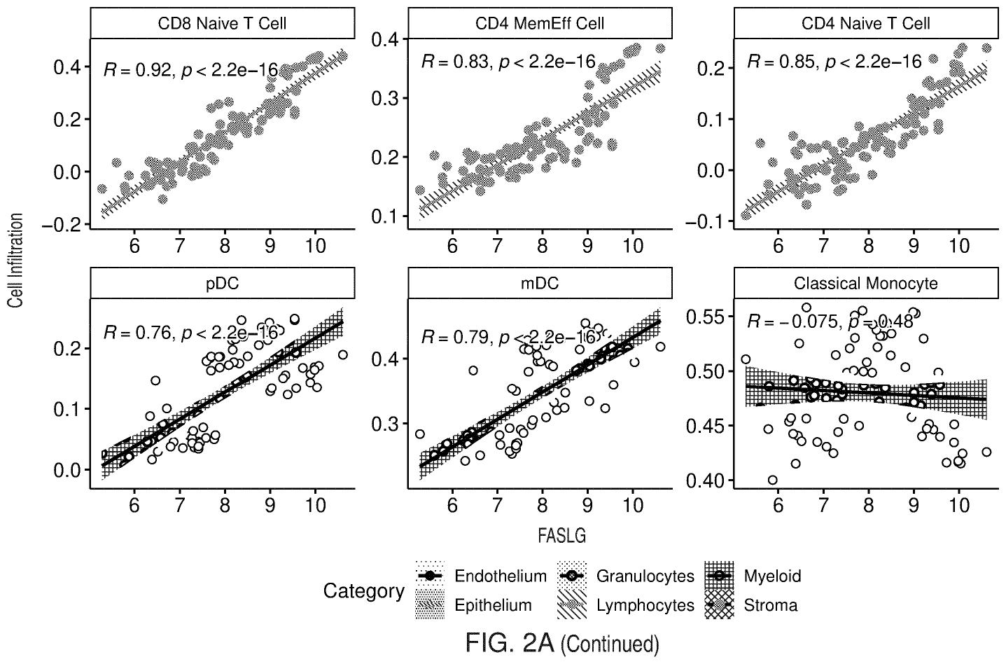

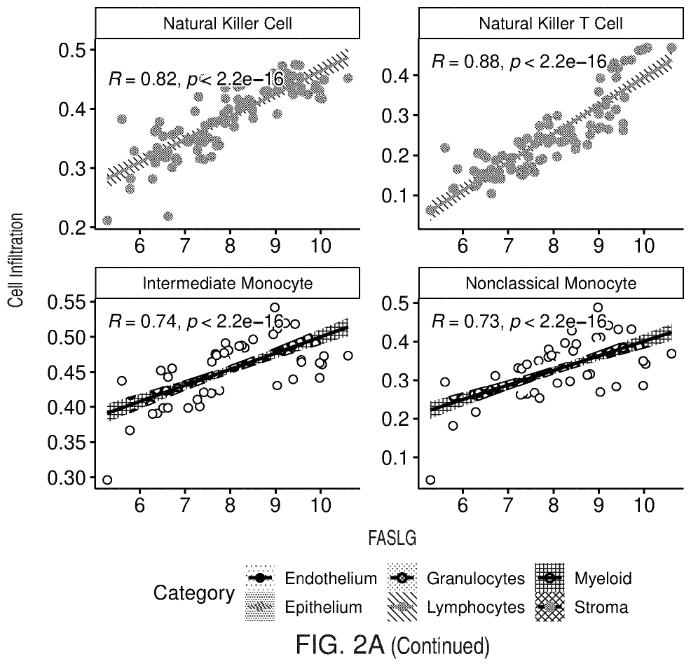

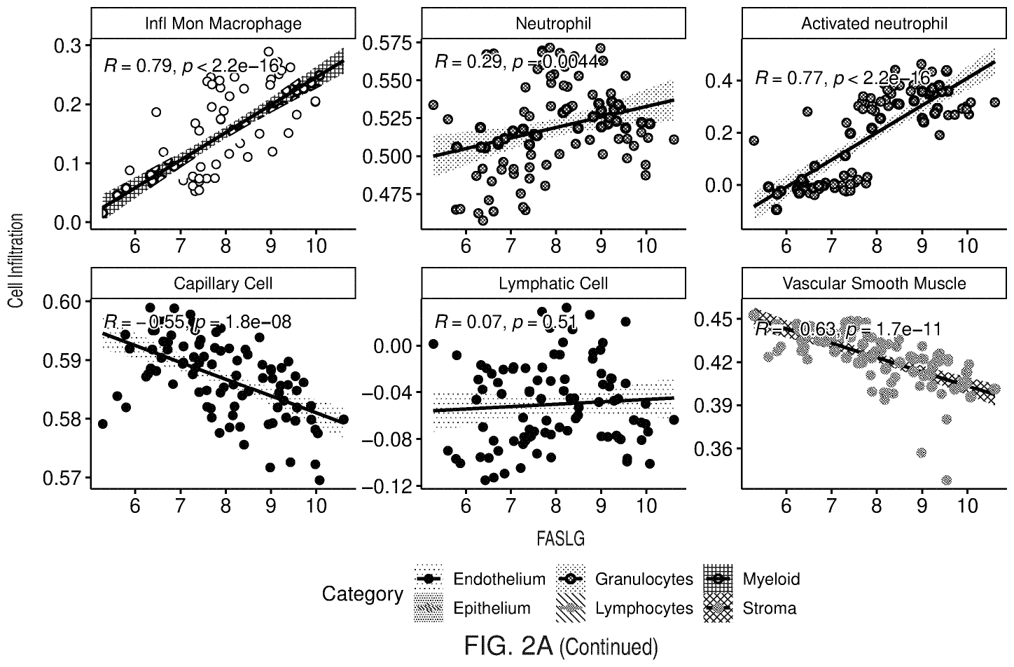

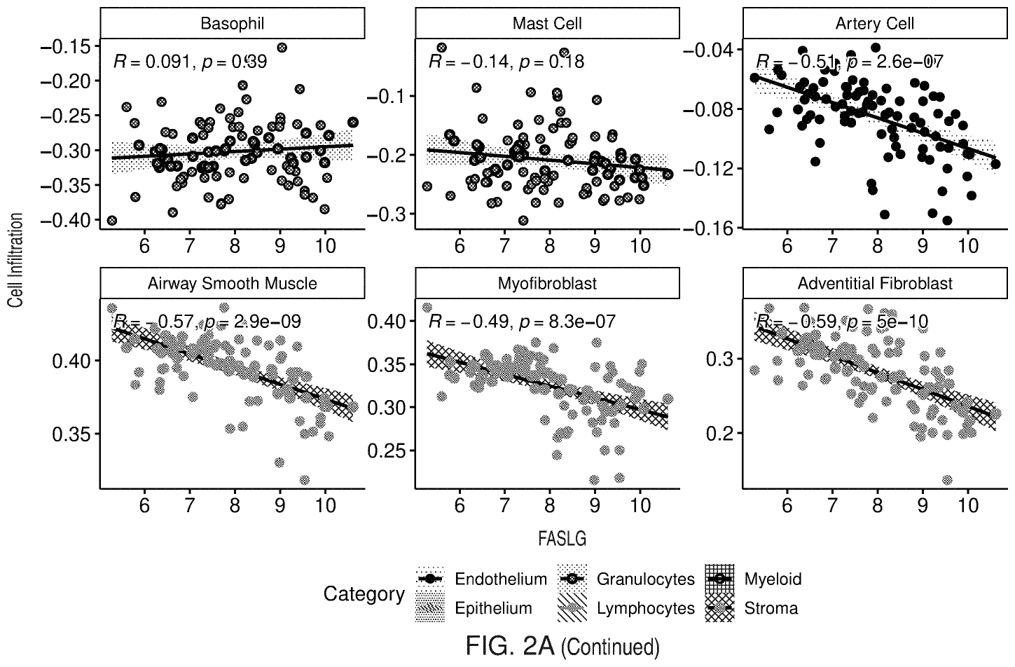

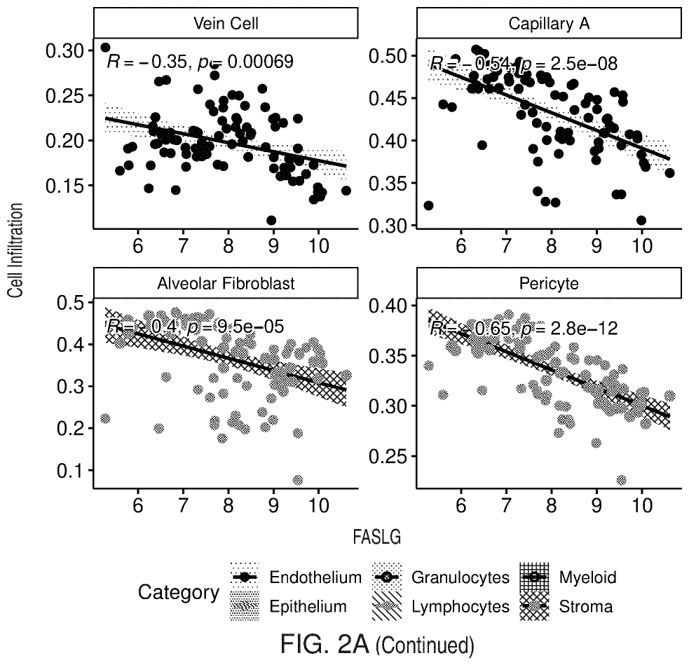

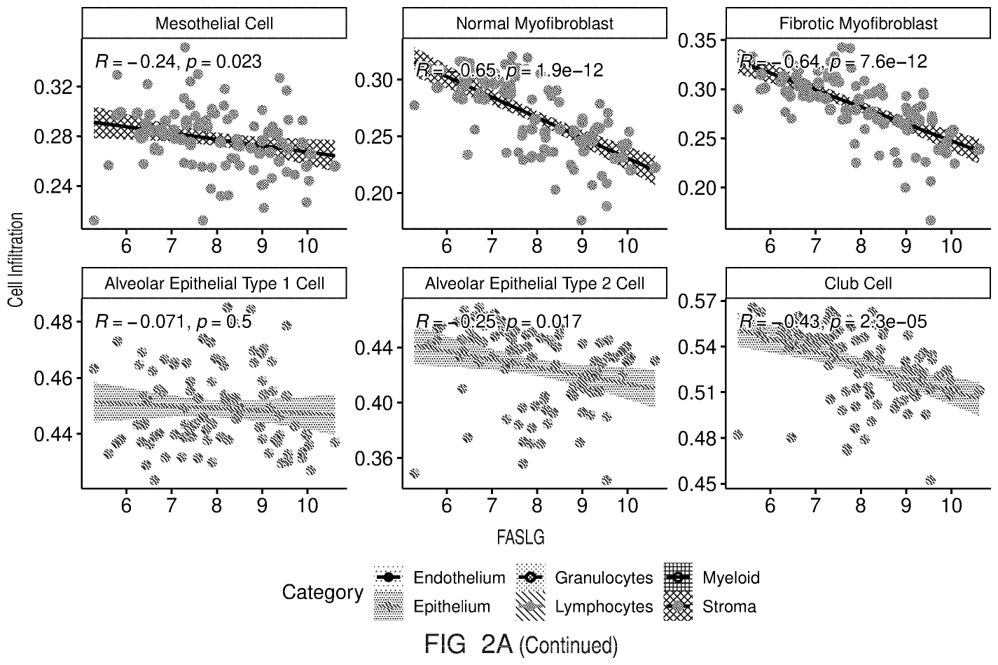

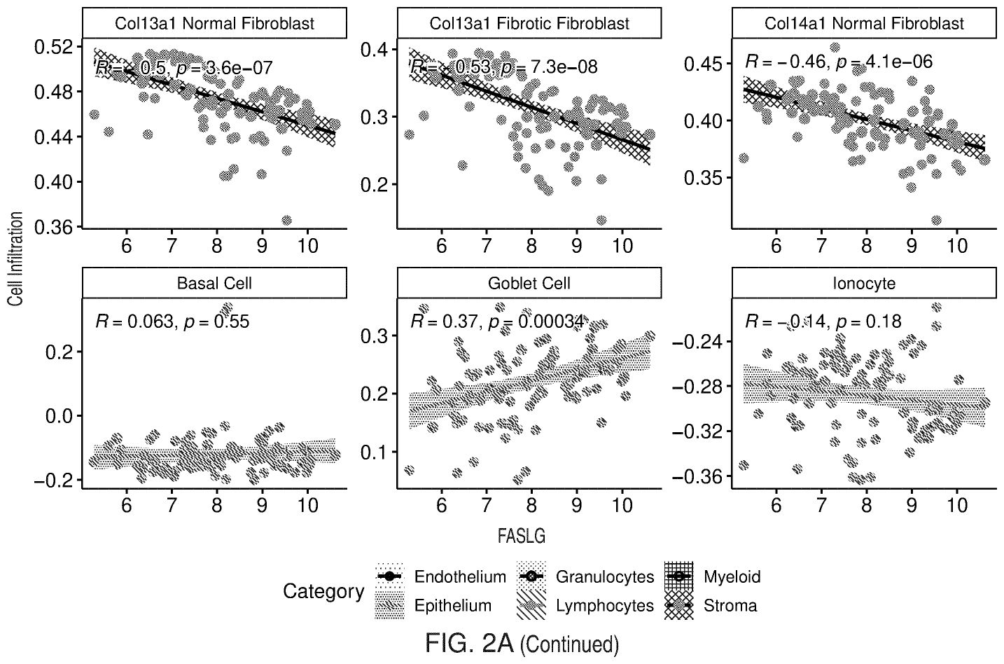

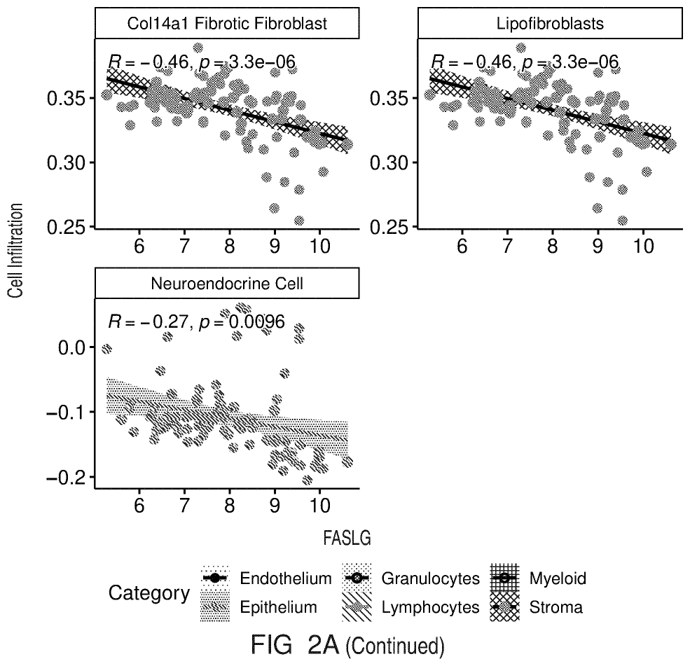

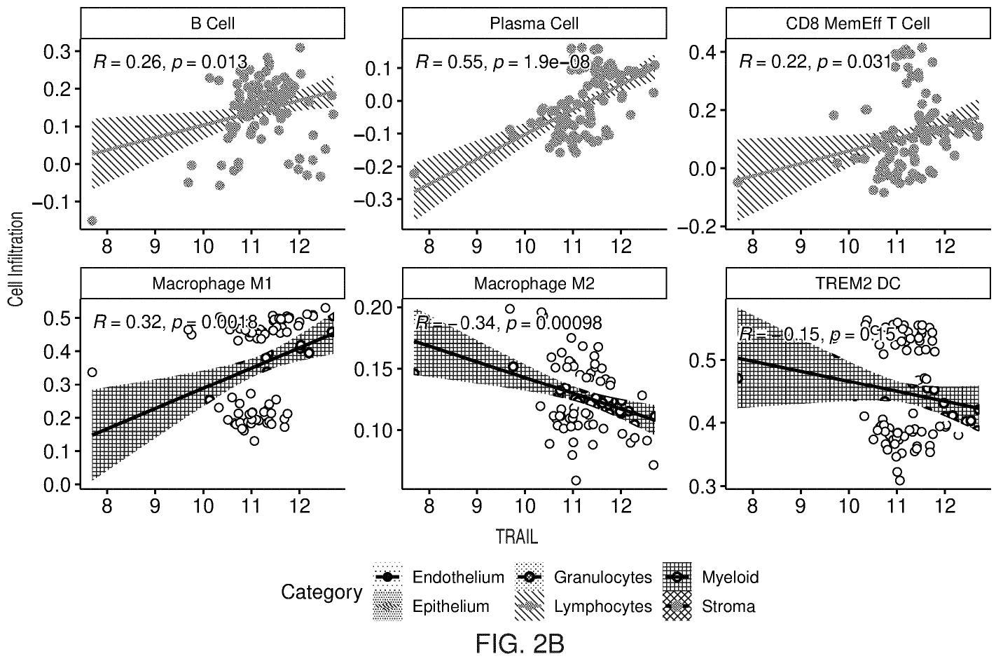

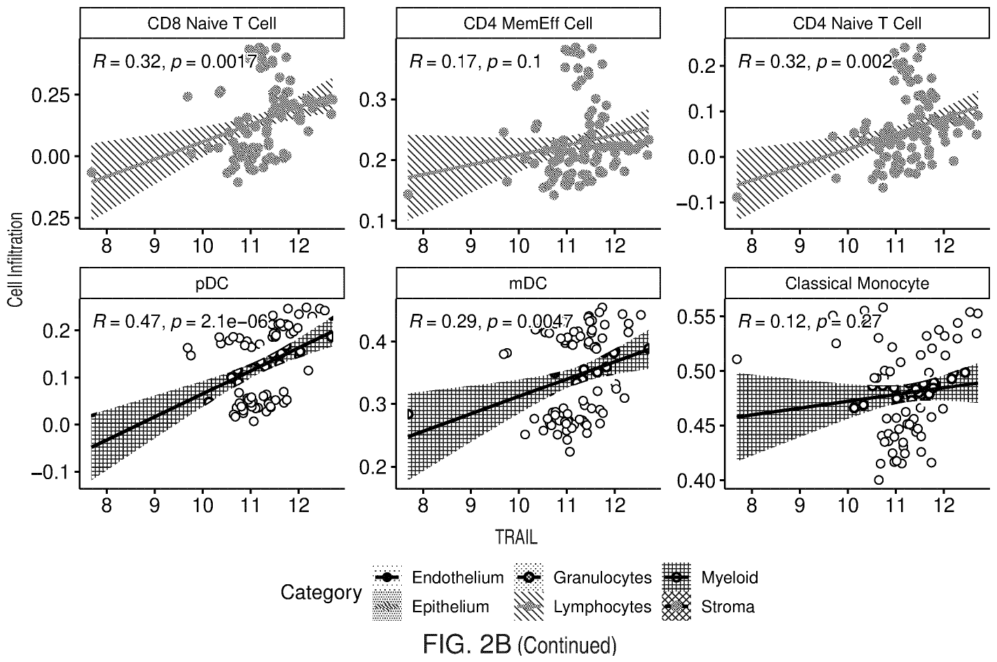

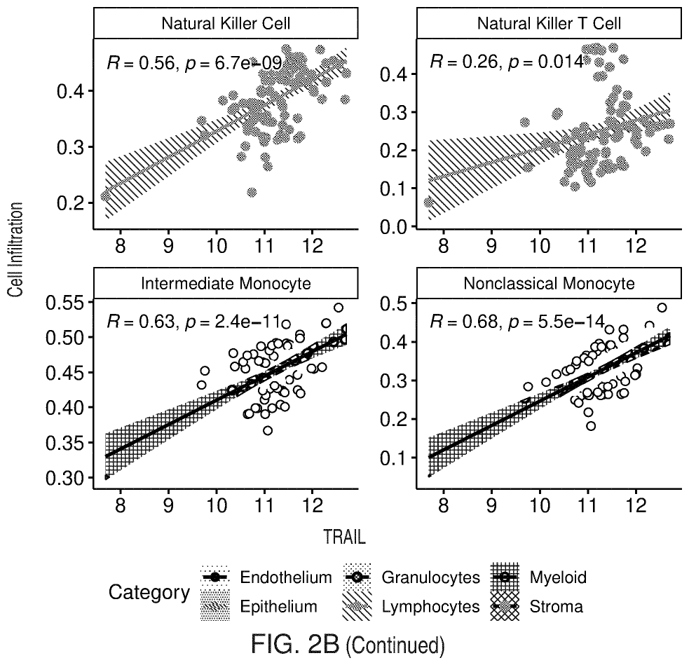

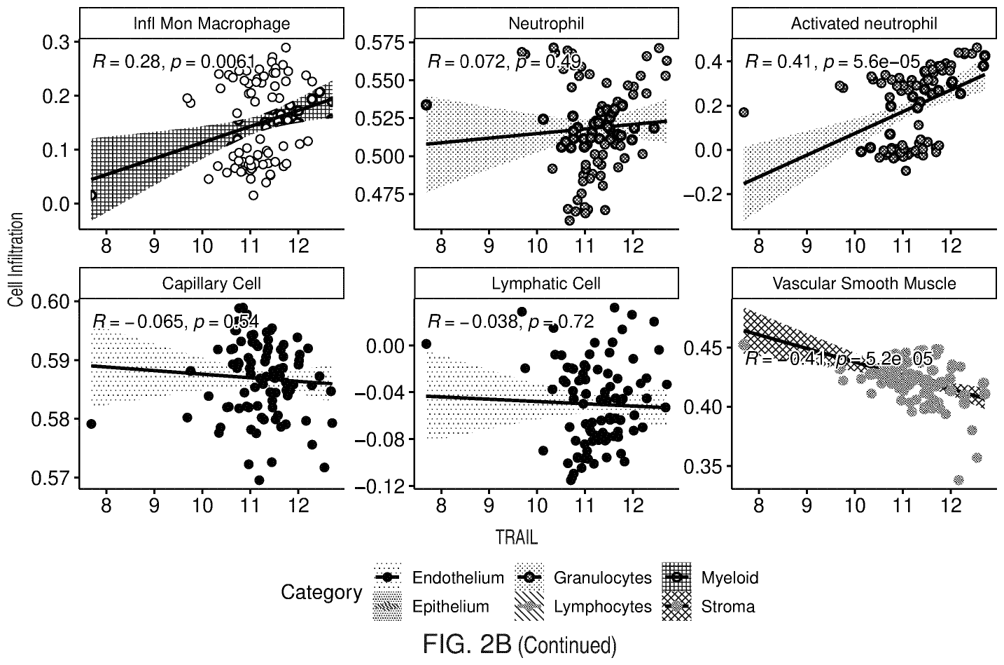

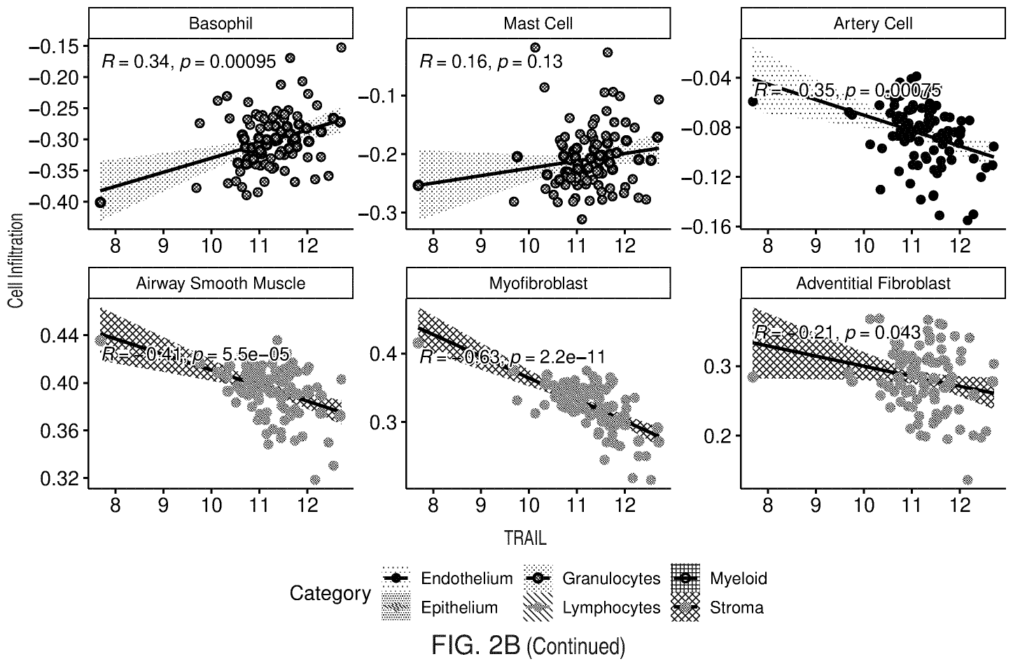

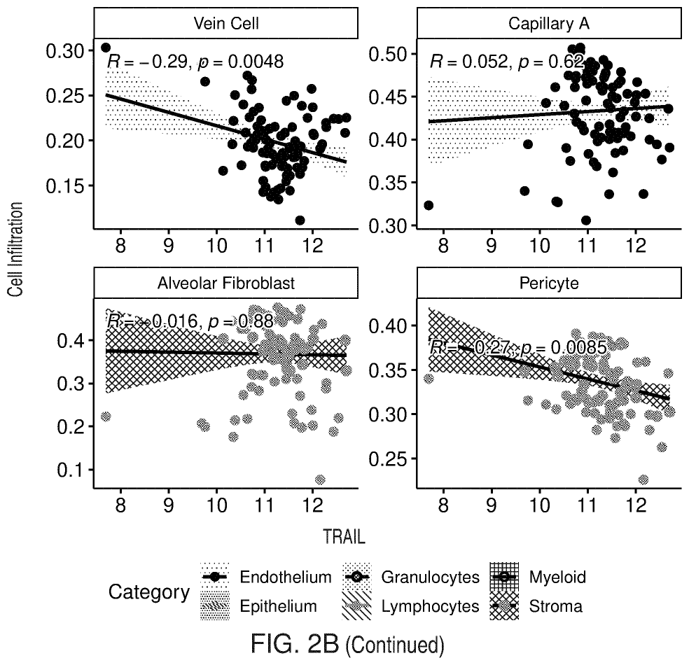

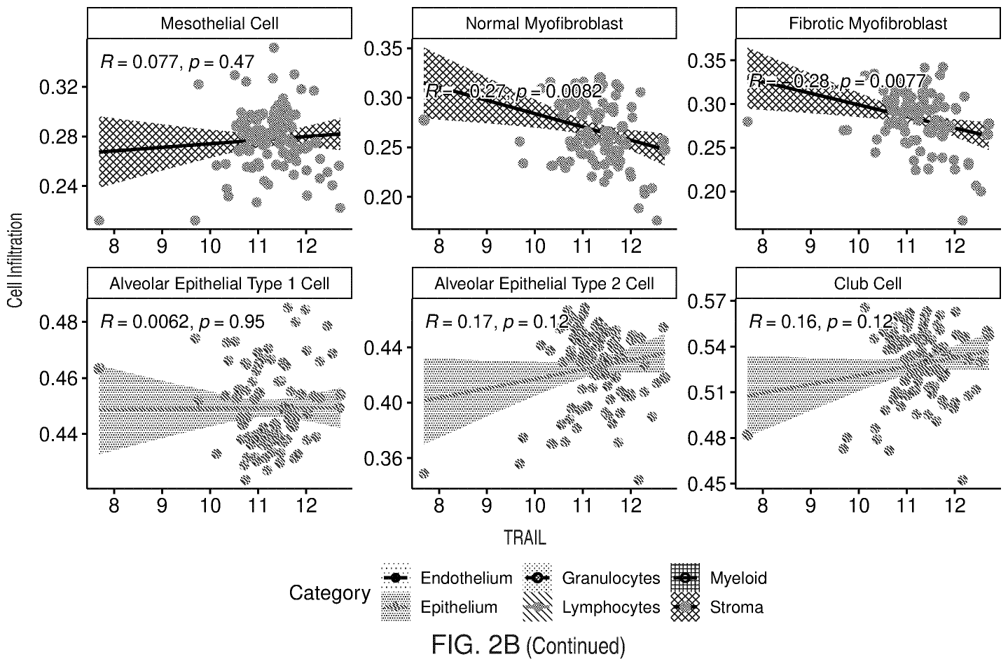

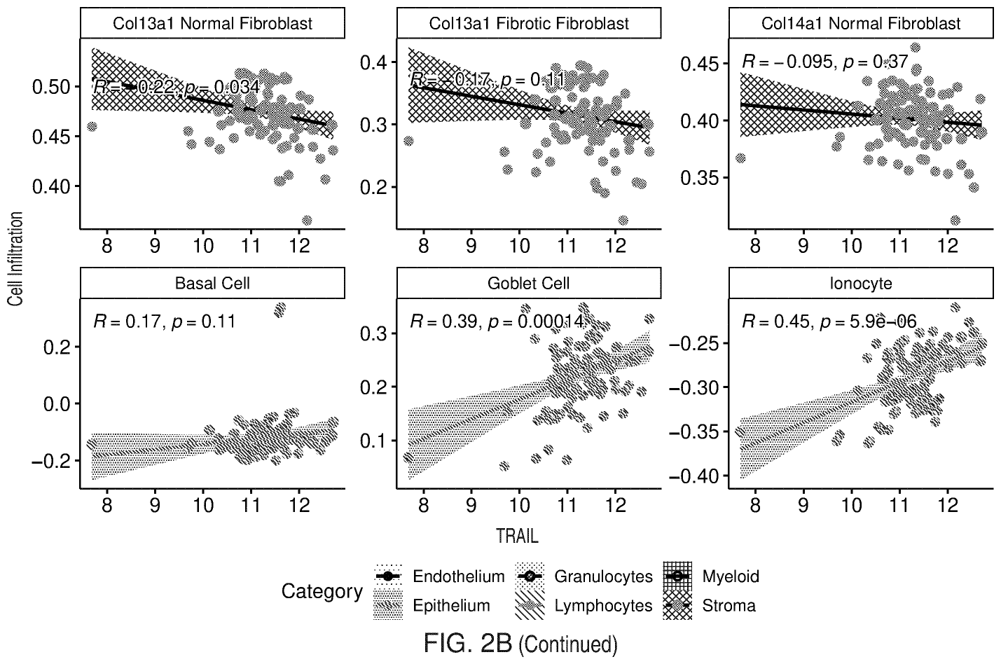

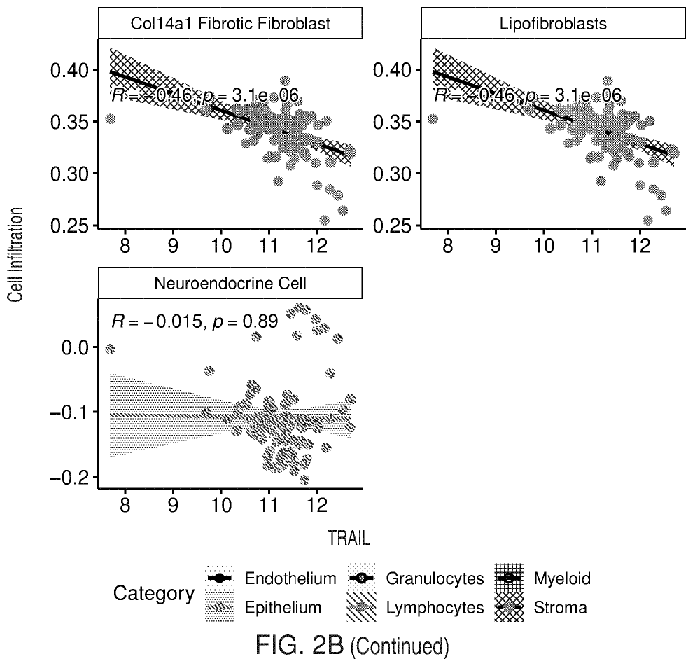

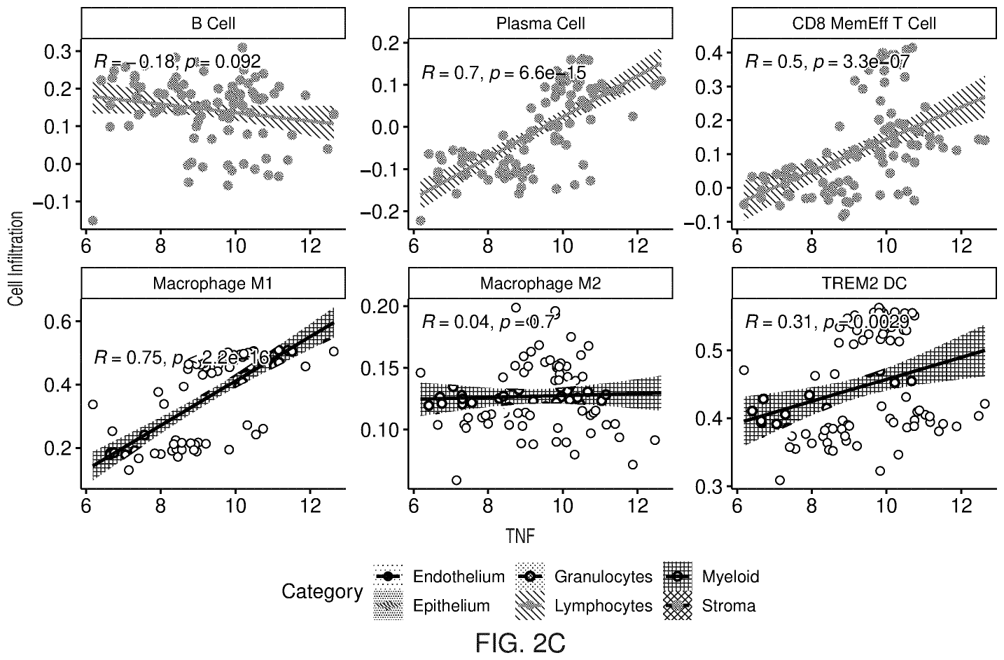

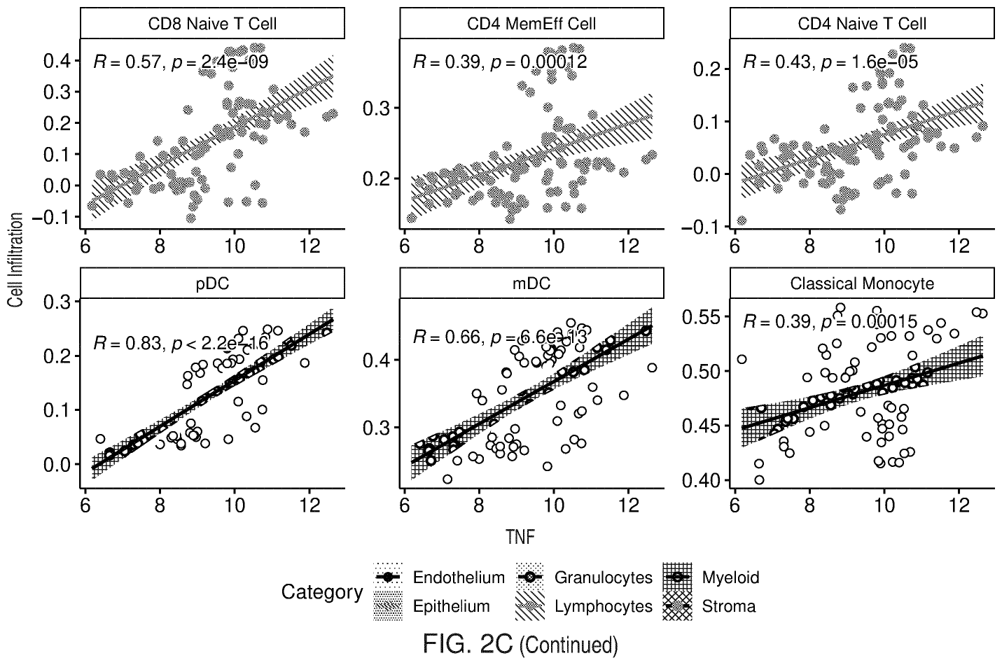

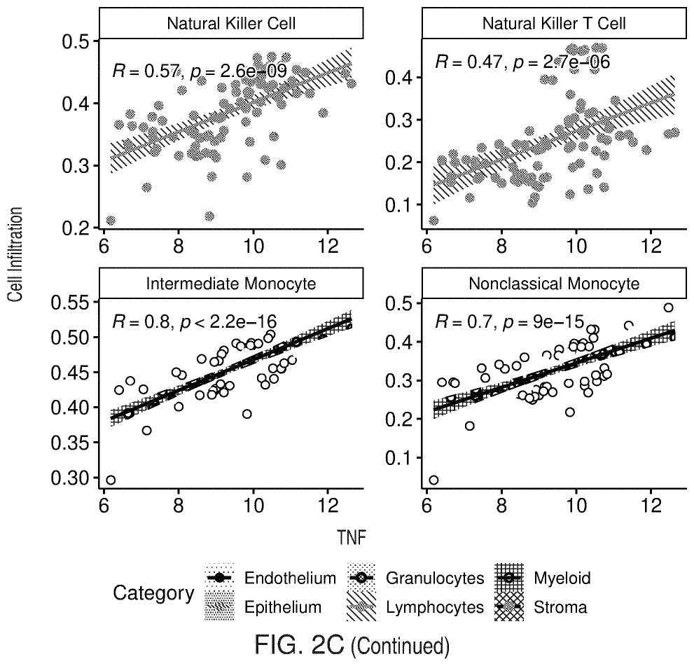

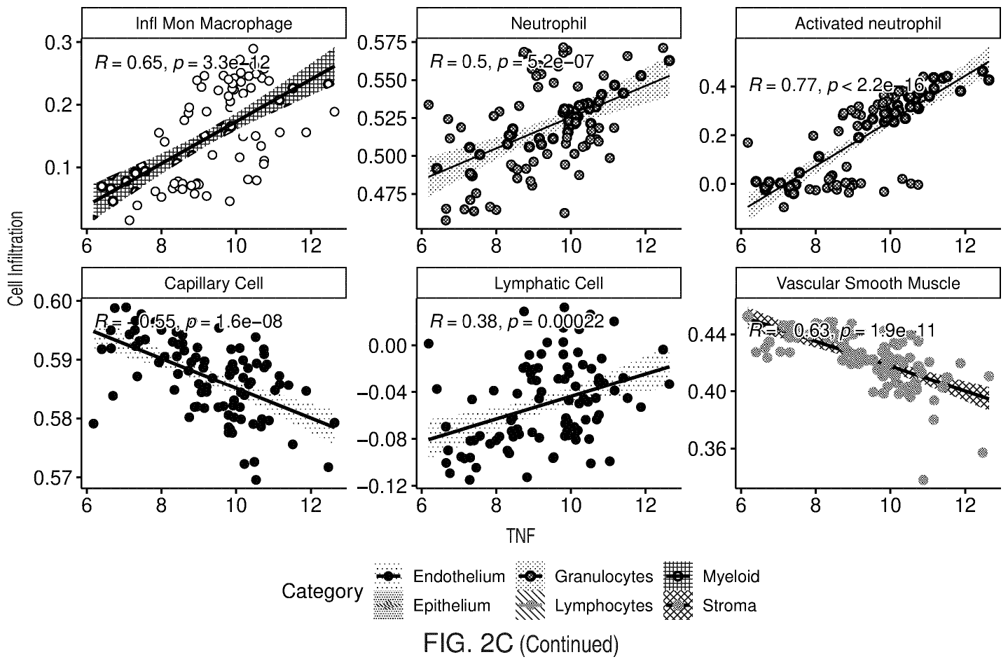

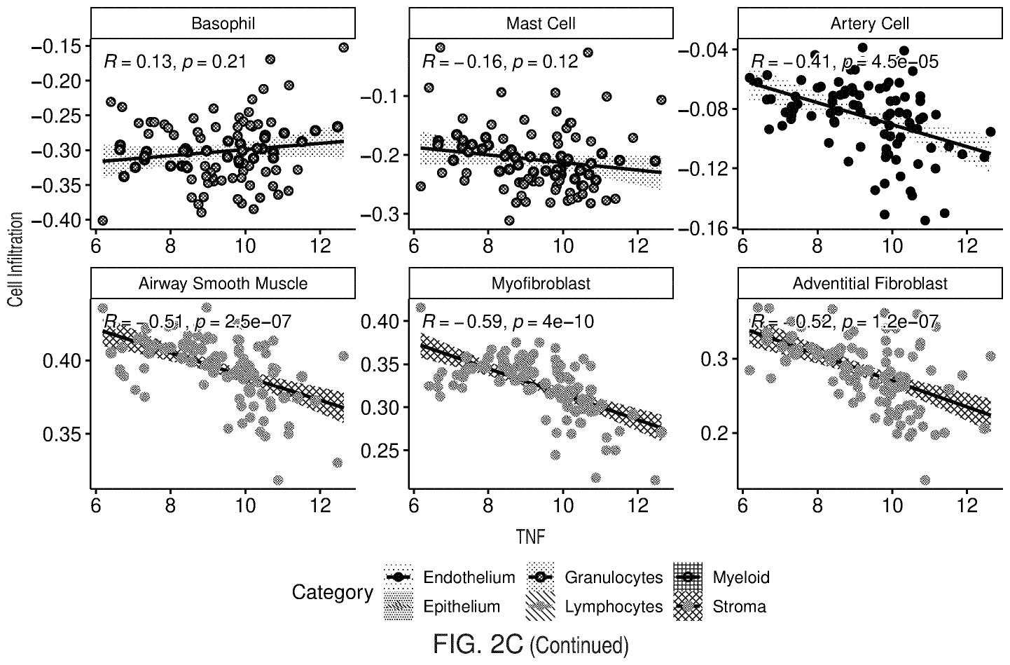

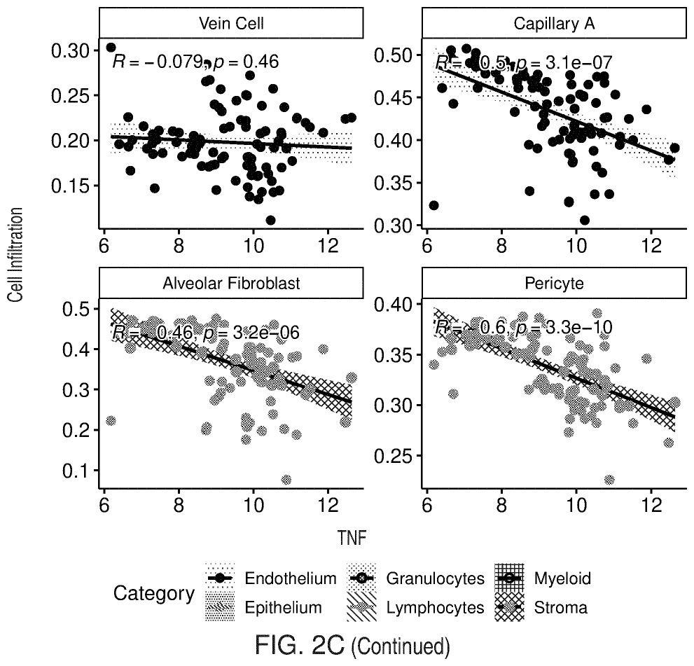

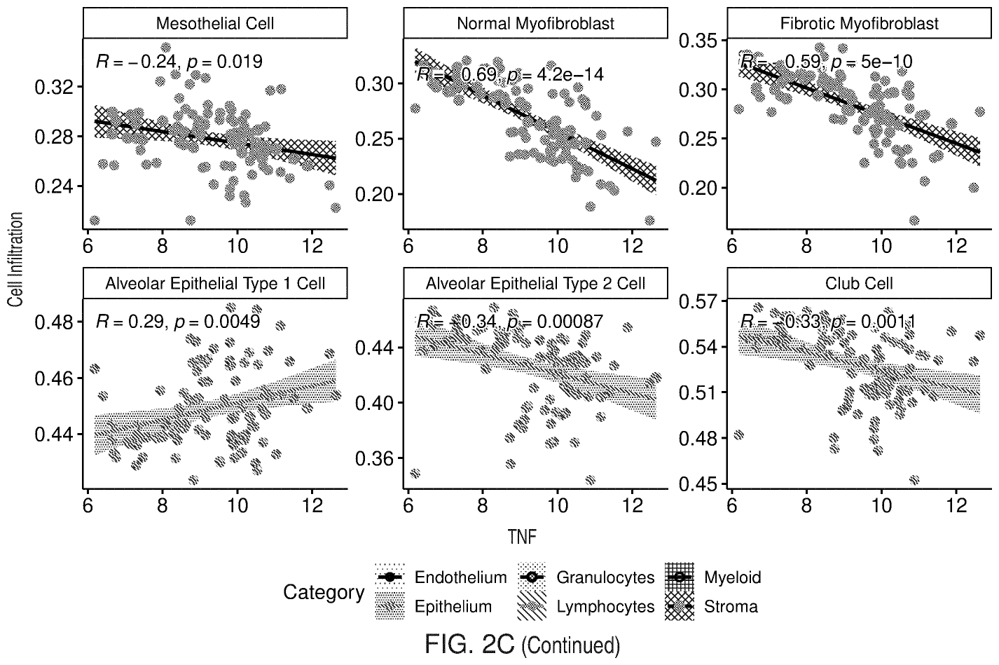

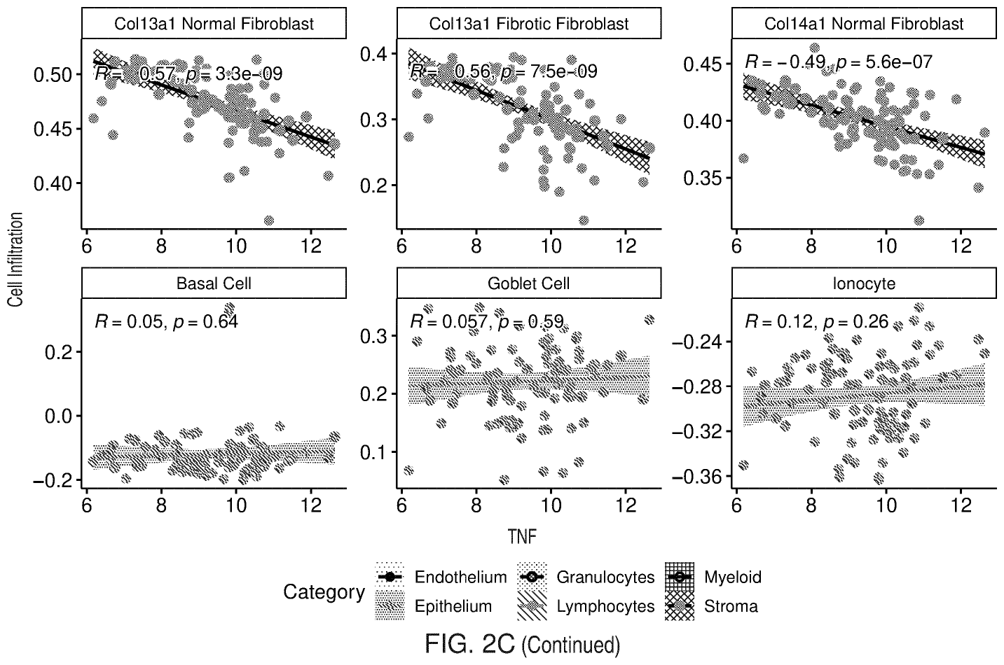

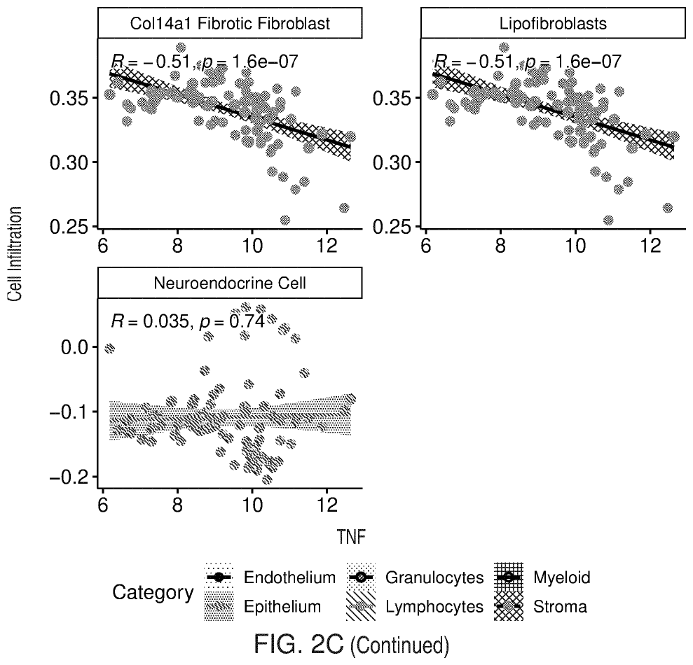

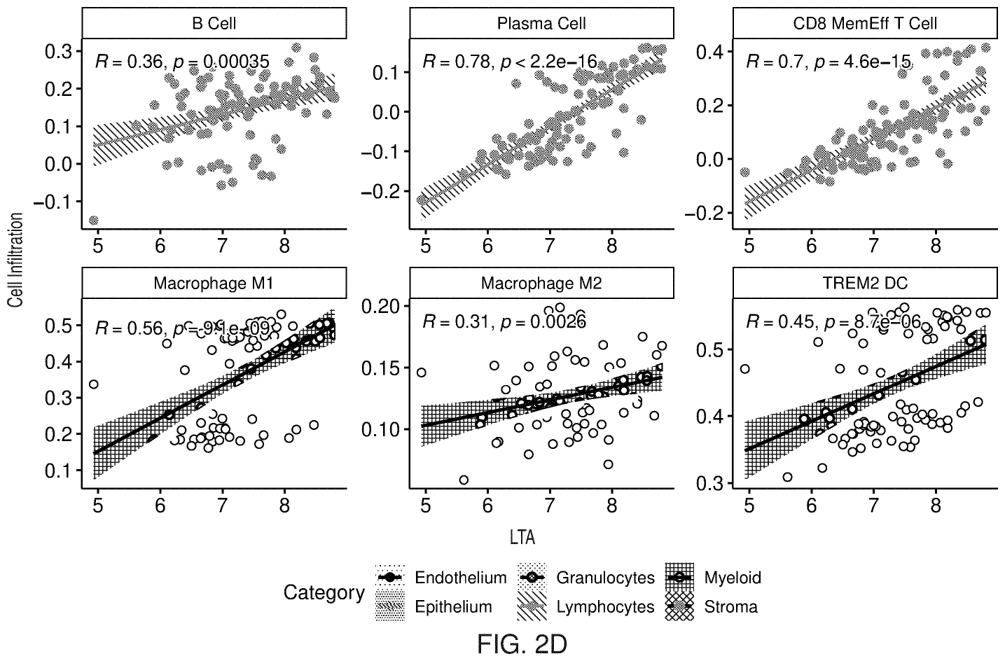

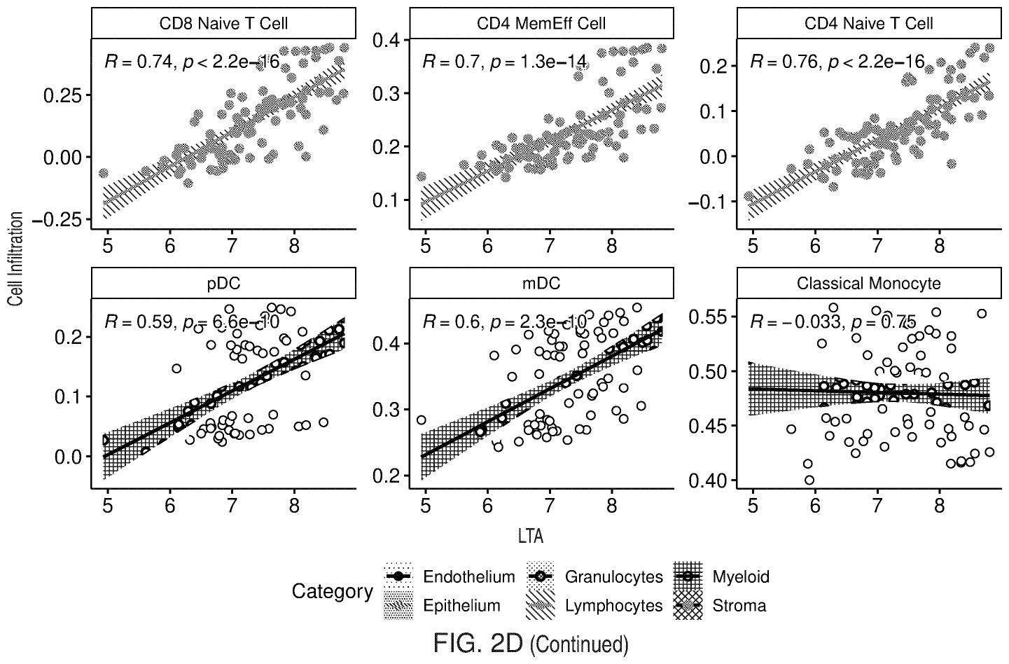

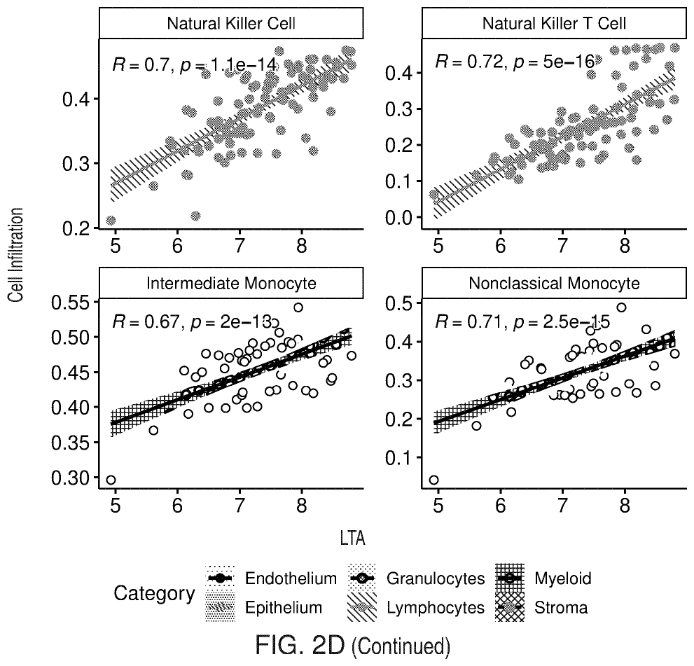

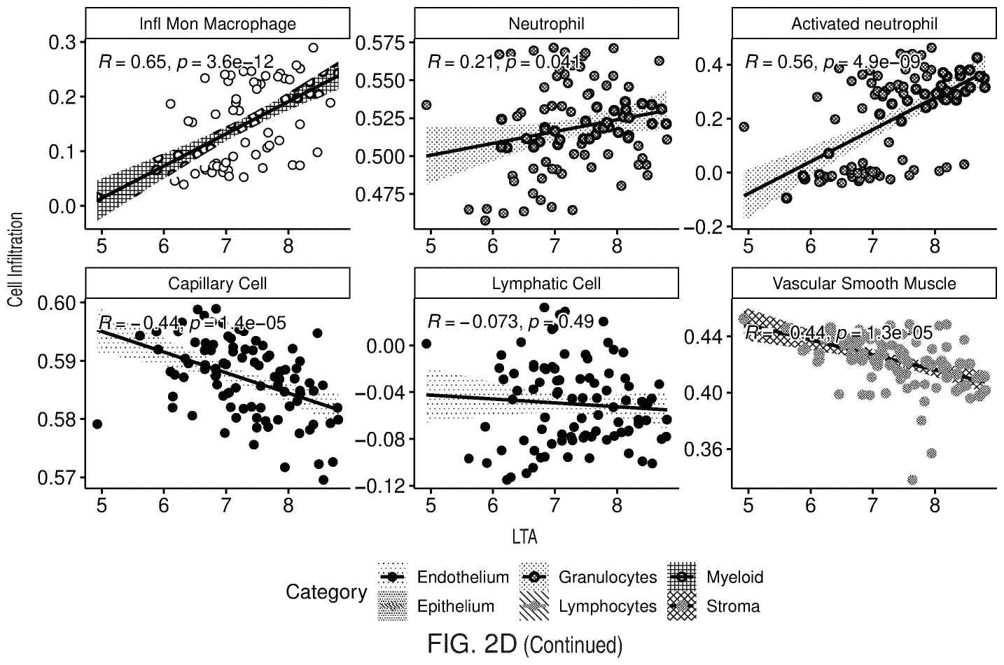

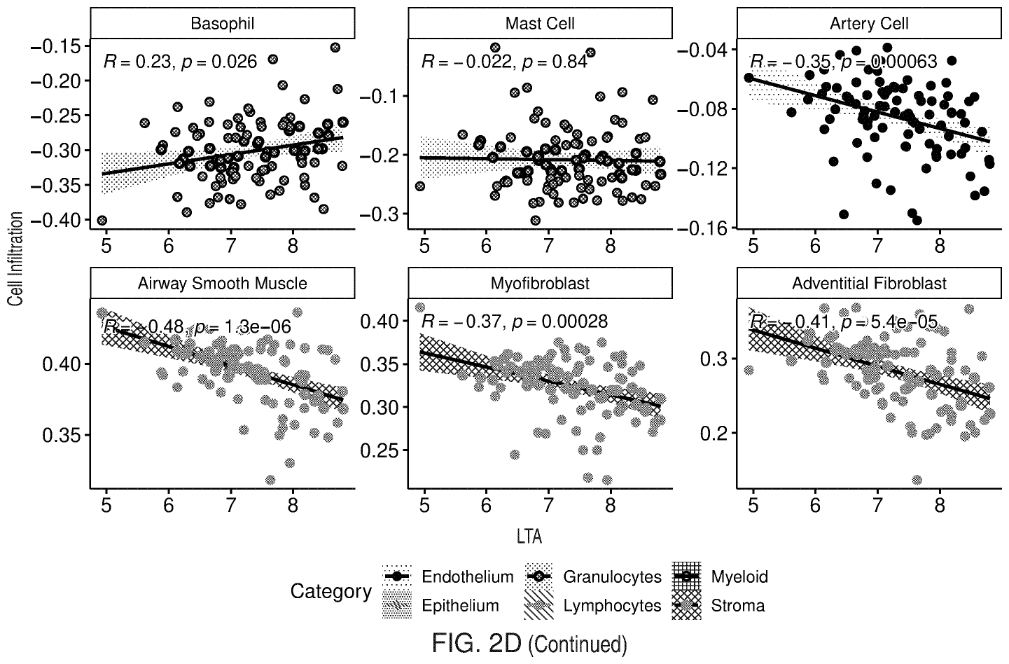

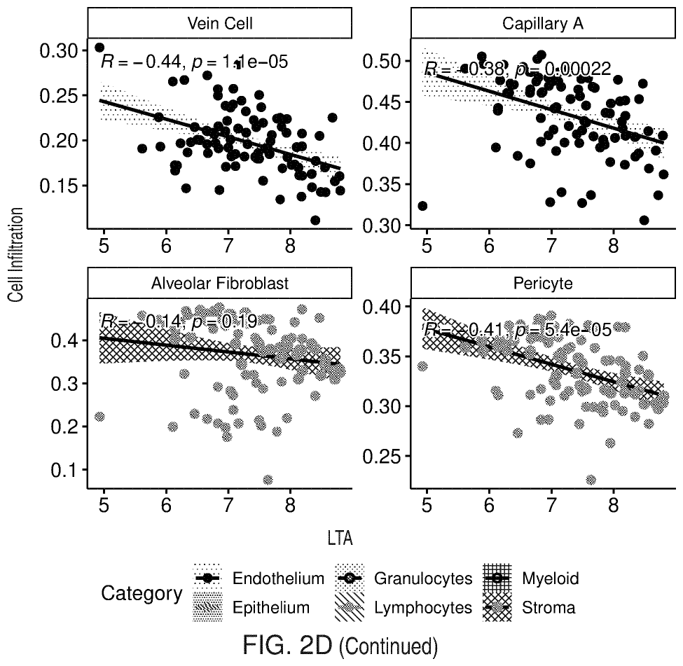

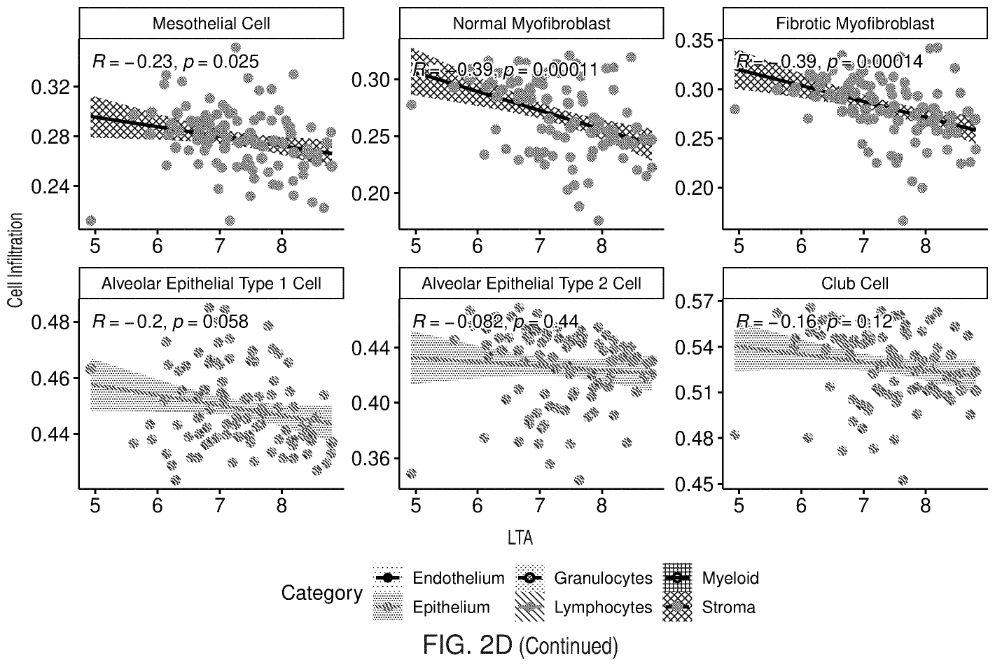

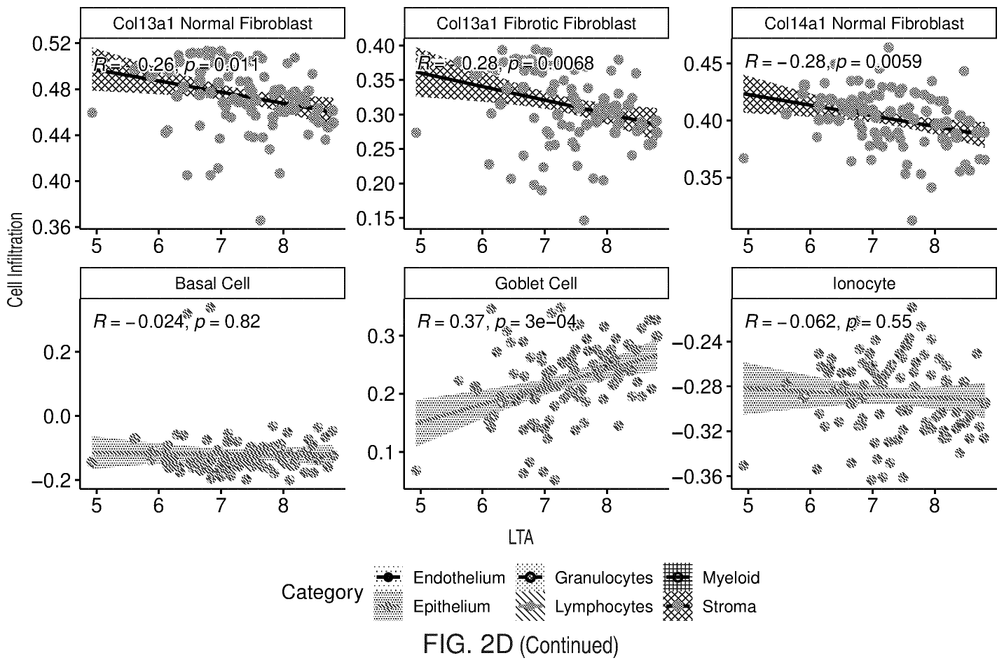

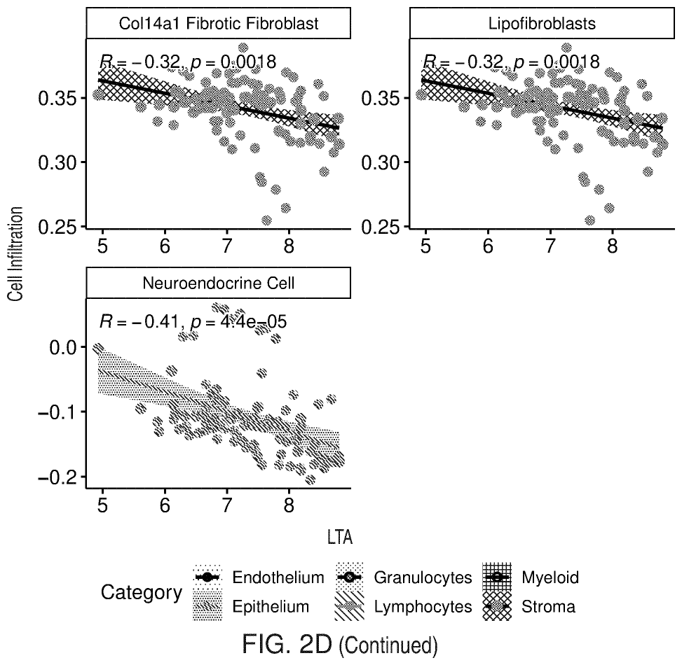

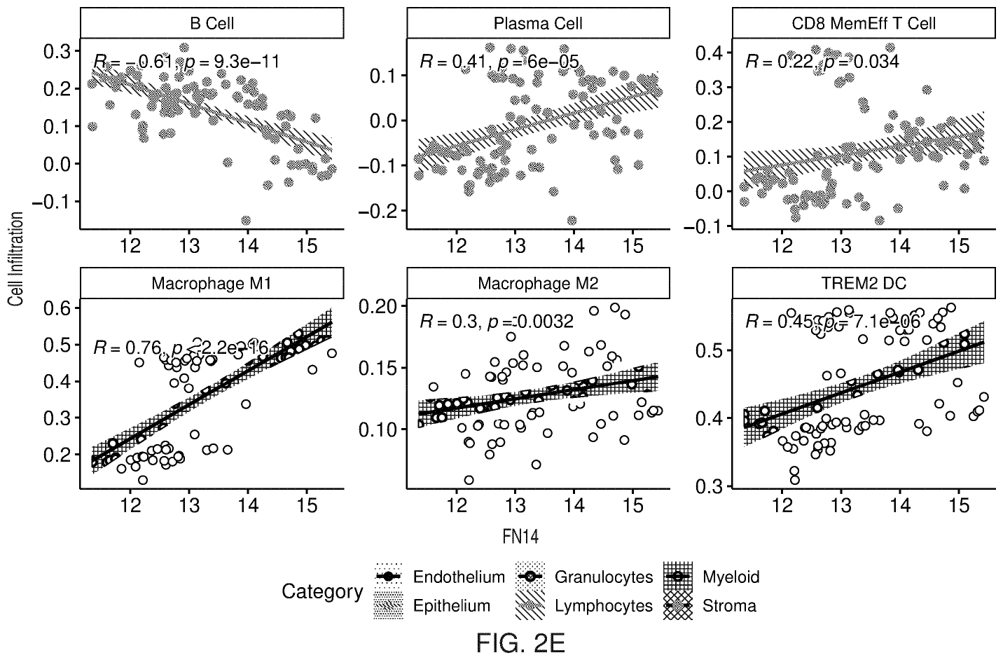

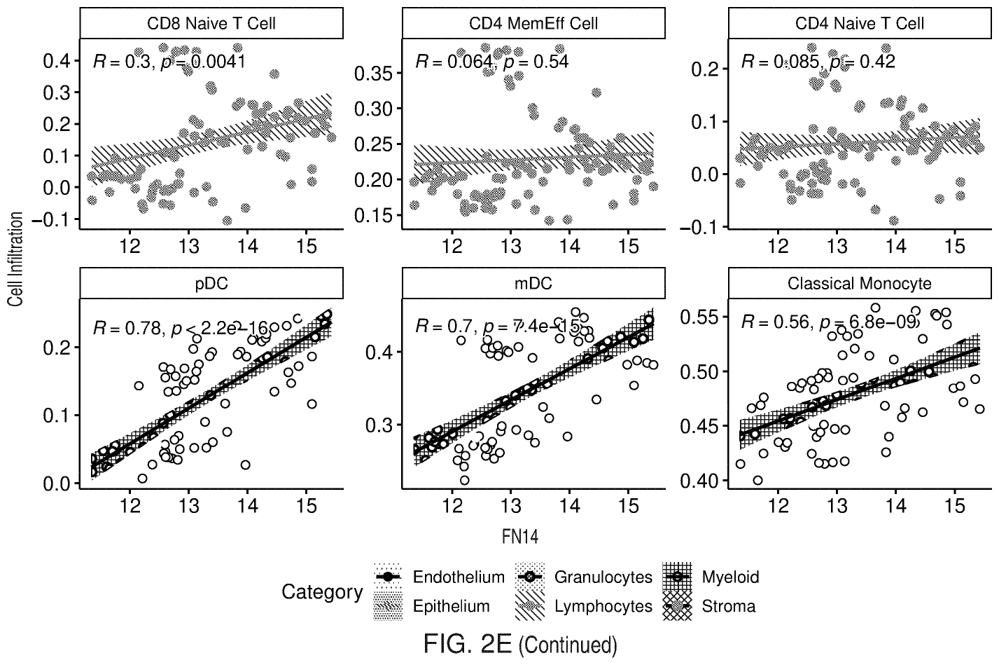

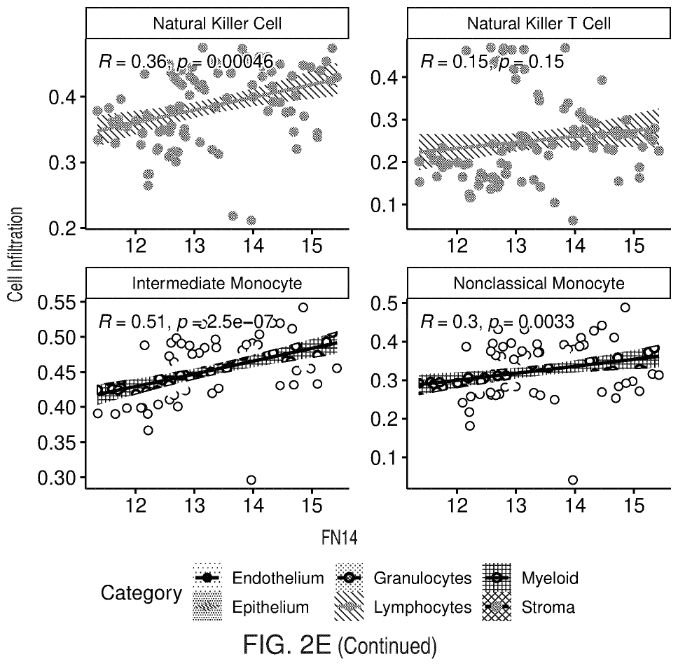

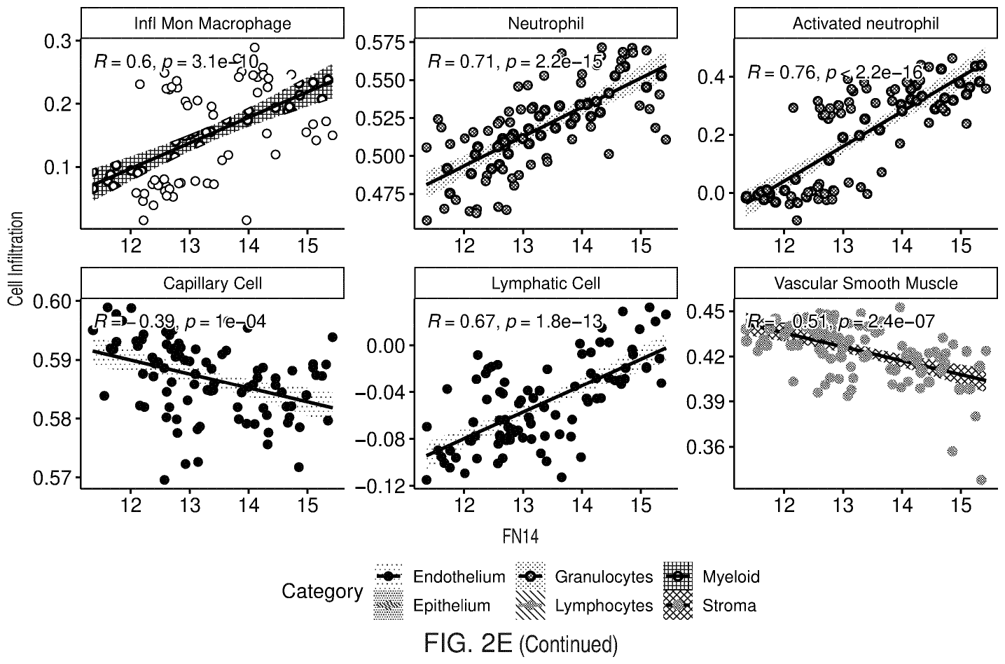

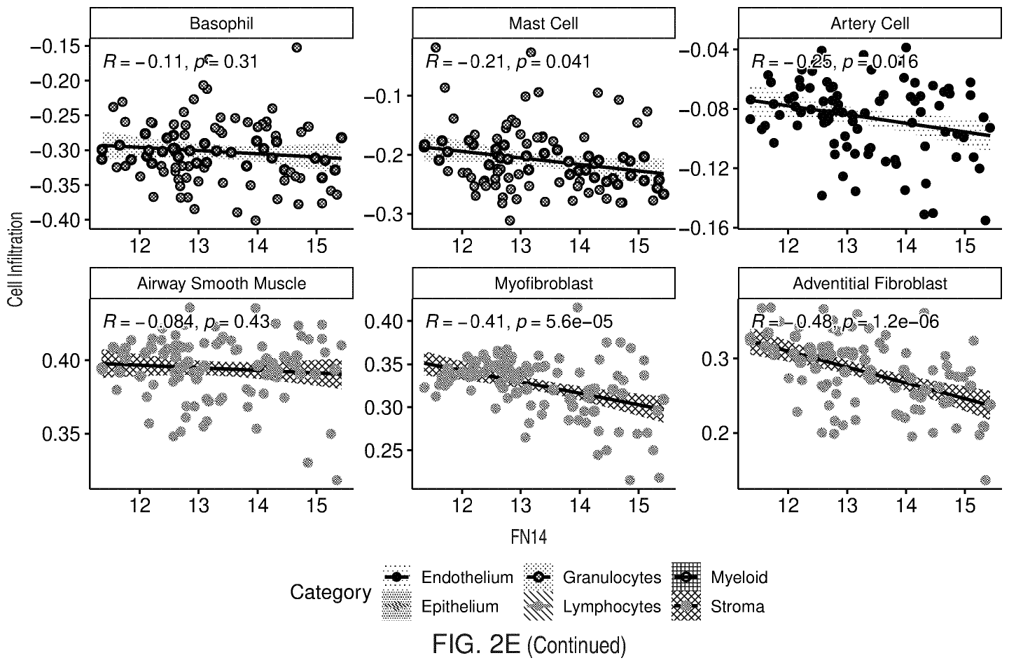

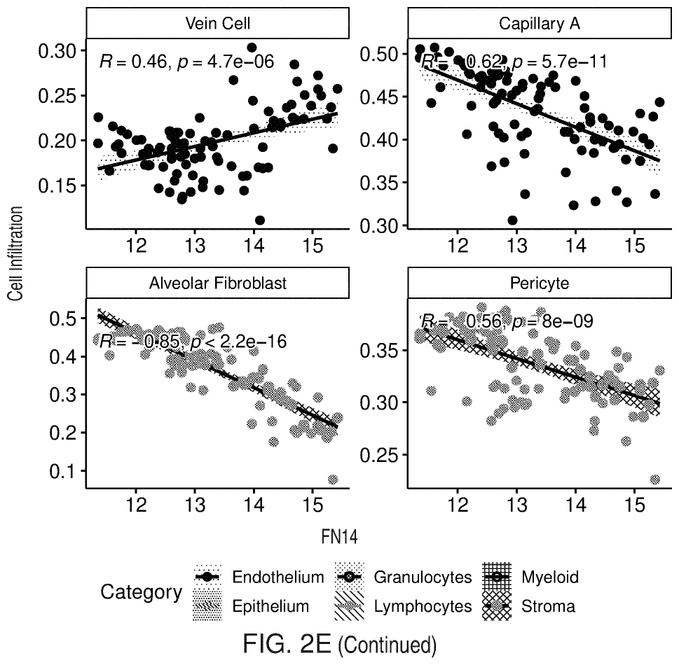

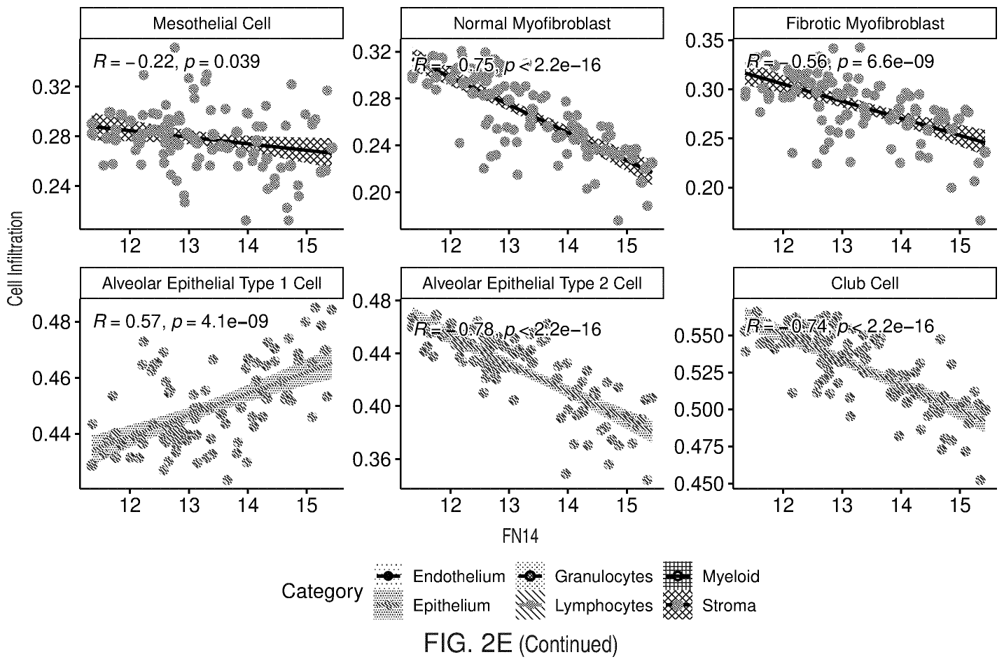

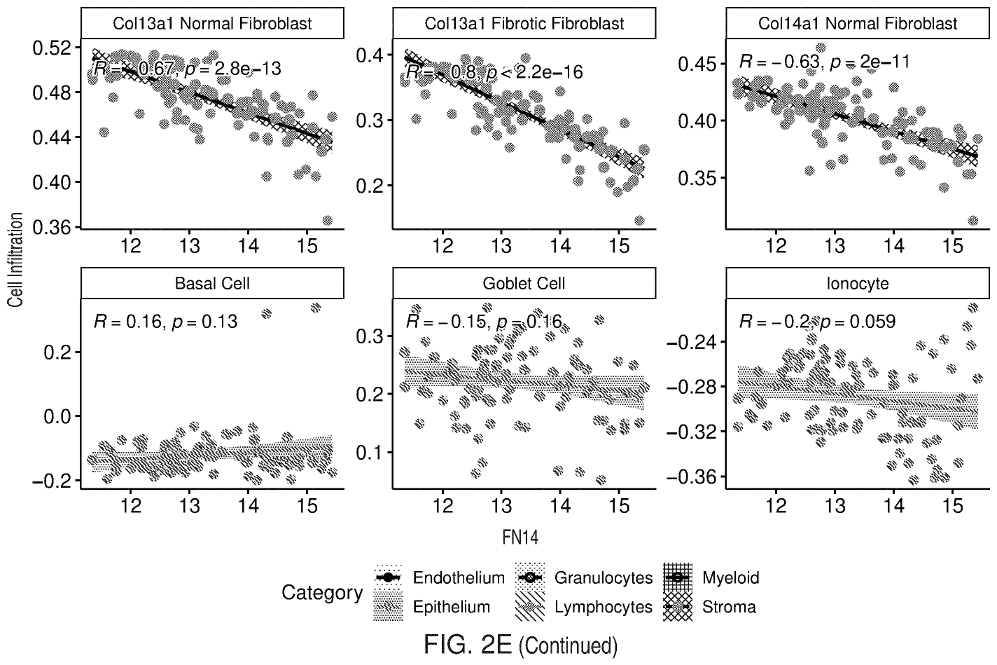

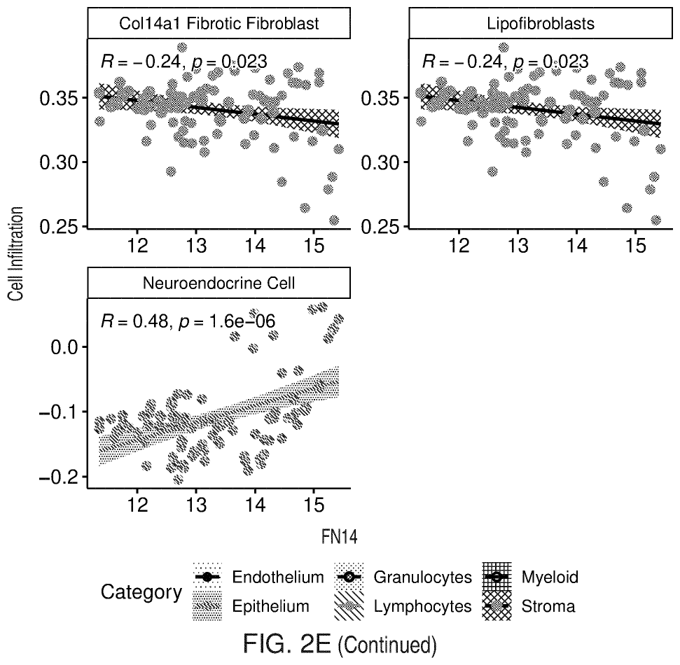

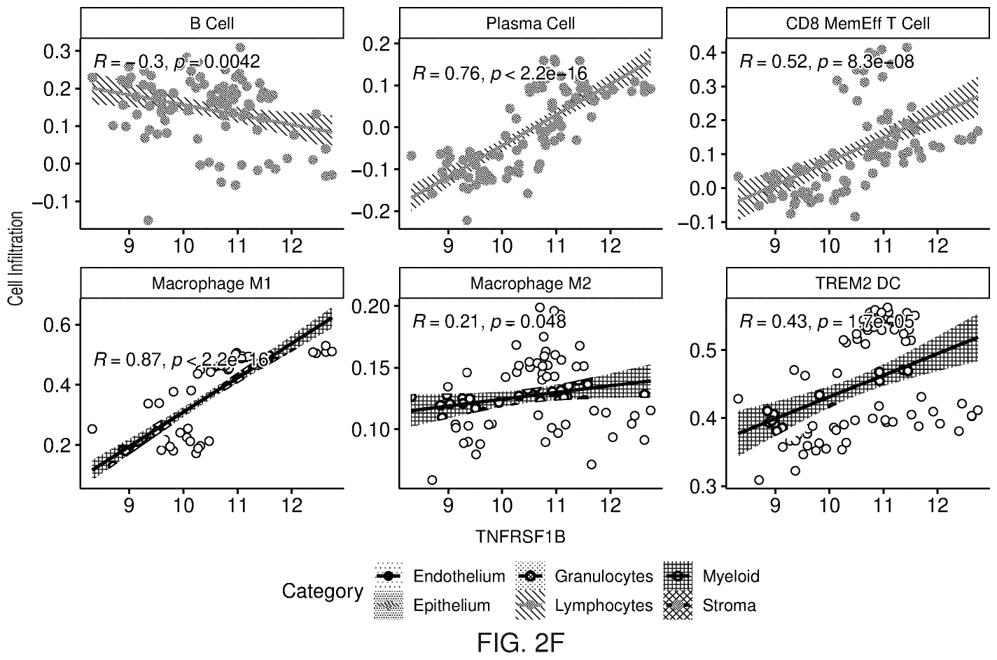

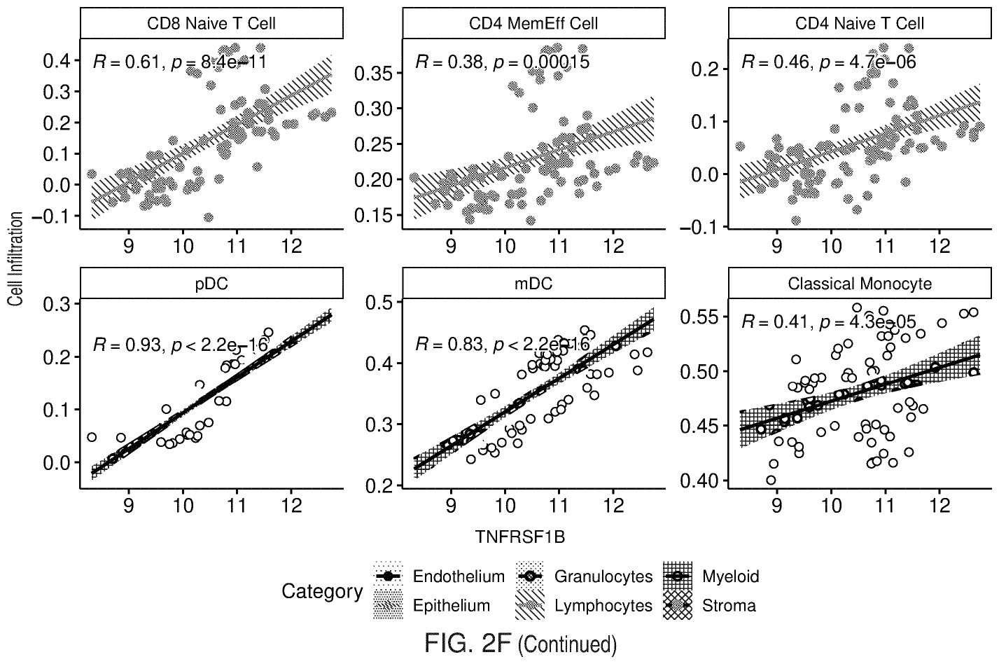

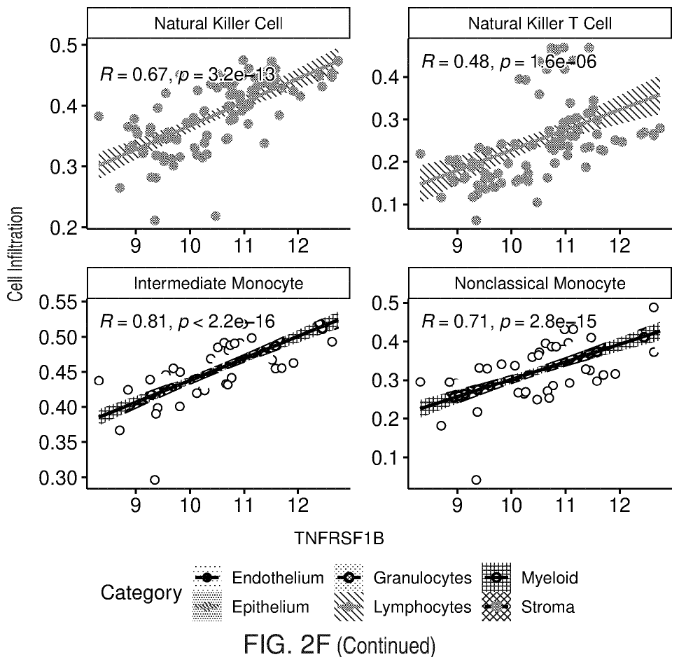

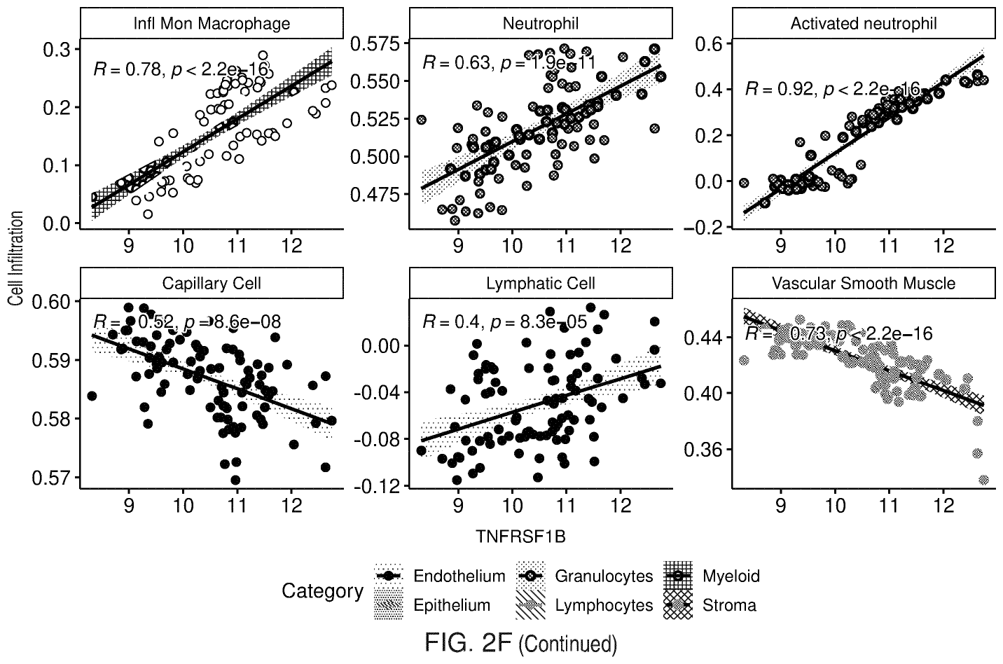

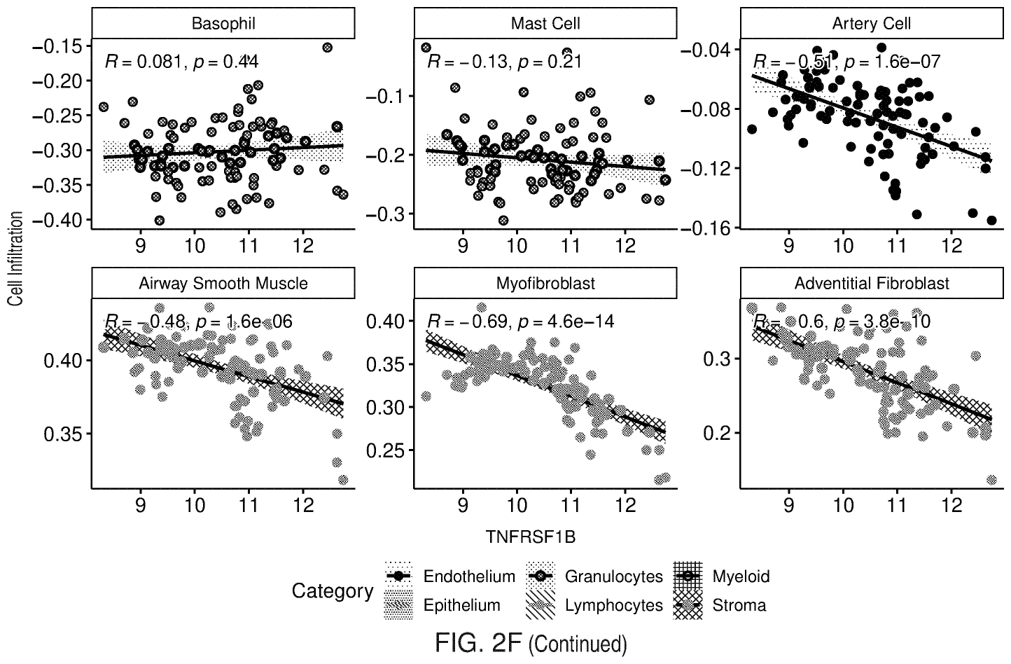

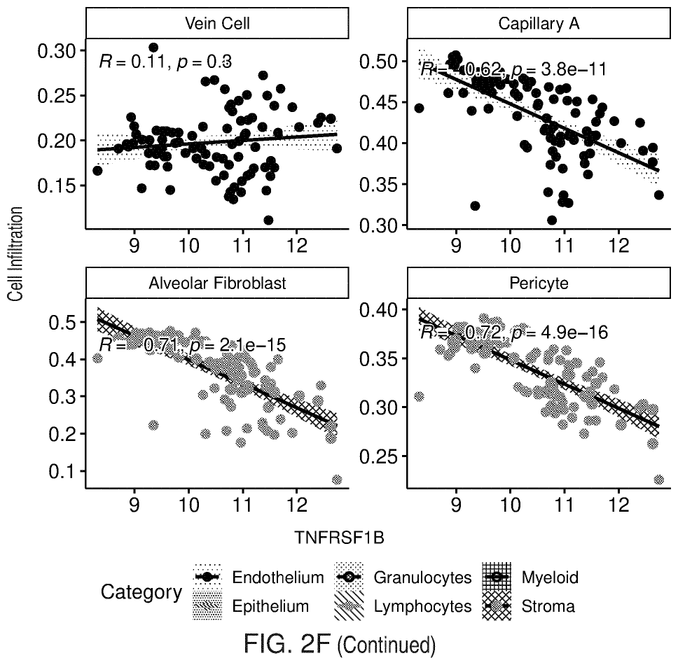

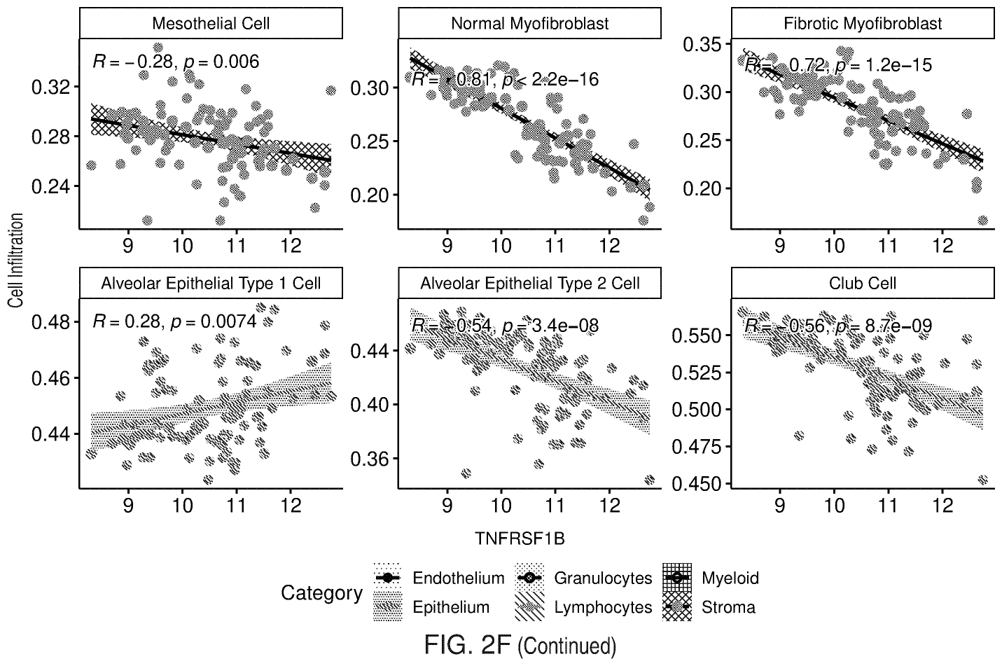

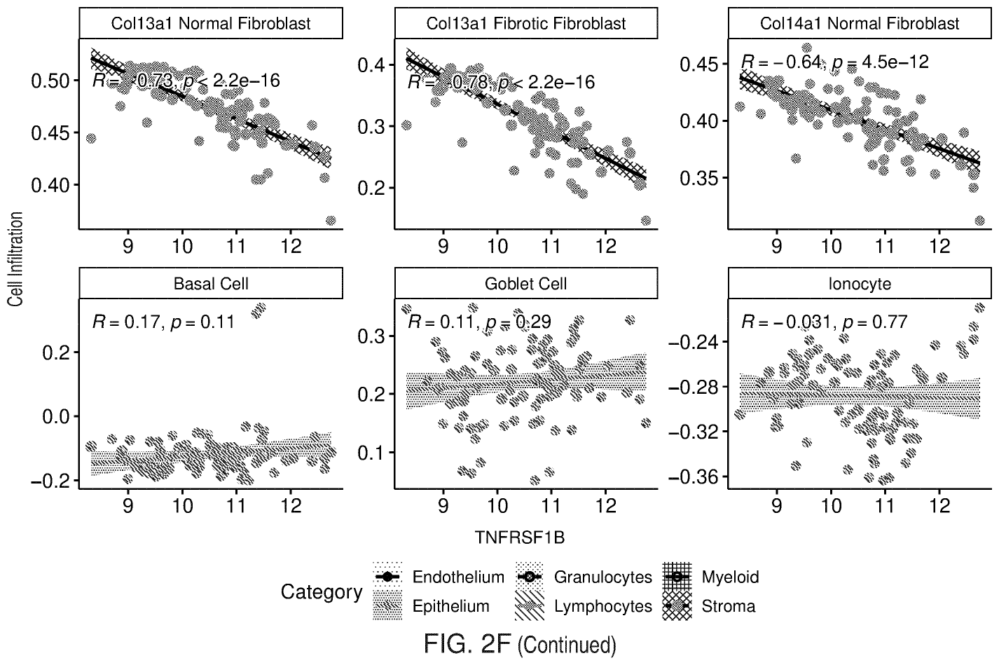

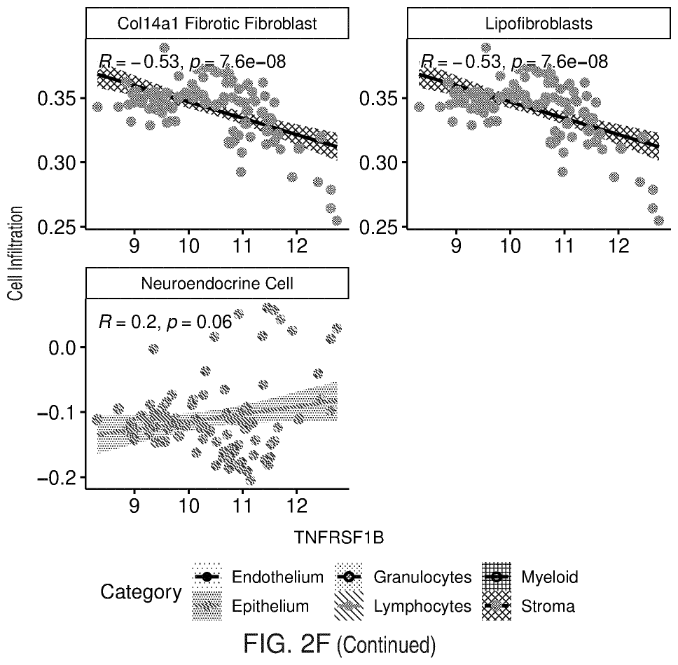

SARS-CoV-MA15 is a version of SARS-CoV(-1) which was adapted to the mouse and to causing an ARDS-resembling pathology in infected mice through 15 rounds of in-vivo infection and consequent mouse adaption of SARS-CoV(-1) (hence the name SARS-CoV-MA15) (Roberts et al. 2007). In 2016, global RNA-Seq expression data of the lungs of mice infected with SARS-CoV-MA15 at different doses (i.e. at 102, 103, 104 or 105 plaque-forming units (pfu)) or mock-infected was obtained at different times following infection, i.e. at 1 day, 2 days, 4 days and 7 days post infection (McDermott et al. 2016). The present inventors have performed a targeted bioinformatic analysis of the expression levels of the death ligands FasL, TRAIL, TNF and LT-alpha and the TNF receptor superfamily members FN14 and TNFR2 (see FIG. 2A for FasL (indicated as FASLG on the bottom of the page), FIG. 2B for TRAIL, FIG. 2C for TNF, FIG. 2D for LT-alpha (indicated as LTA), FIG. 2E for FN14 and FIG. 2F for TNFR2 (indicated as TNFRSF1B)). Importantly, this analysis was performed in a way that allows for the deconvolution of immune and non-immune cell types which means that the expression level of a given gene (or group of genes) can be correlated with the presence of (a) particular cell type(s) in the lungs of each one of these individual mice, importantly, including with an increased or decreased presence of such (a) cell type(s). The cell types analysed in this way include different types of immune cells as well as non-immune cell types such as, e.g., but not limited to, different types of epithelial and endothelial cells as well as other cells such as pericytes, vascular and airway smooth muscle cells and different types of fibroblasts.

This analysis revealed that the amount of mRNA encoding TRAIL detected in the lungs of infected mice positively and highly significantly correlates with the presence of lymphocytes, such as plasma and NK cells as well as with myeloid cells and granulocytes, such as pDCs and activated neutrophils, respectively (FIG. 2). Interestingly, the amount of TRAIL-encoding mRNA also positively correlates, with high statistical significance, with a decrease in, e.g. endothelial and stromal cells such as arterial cells and airway smooth muscle cells and fibroblasts, e.g. myofibroblasts, respectively (FIG. 2). Hence, the majority of the cell types whose presence correlates with increased expression of TRAIL are of immune cell origin whereas those whose decreased presence correlates with increased TRAIL expression are exclusively non-immune-cells. These results suggest the implication of TRAIL expressed by one or more of the immune cell subsets in the killing of one or more of the endothelial and/or stromal cell types whose decrease correlates with increased presence of TRAIL and the immune cell types that express it.

Examining the same mRNA expression data set as above for TRAIL, the present inventors have also analysed which cell types were enriched or decreased in the lungs of SARS-CoV-MA15-infected mice and how an increase or decrease in any of these cell types correlated with the presence of FasL mRNA in the lung of each individual SARS-CoV-MA15-infected or mock-infected mouse.

### Example 3: Effect of Different Types of Interferons on Death Ligand-Induced Cell Death on Mouse Fibroblasts

To further validate the potential involvement of TRAIL, FasL, TNF and/or LT-alpha in cell-autonomous and/or immune-dependent death of different types of cells in the lungs of SARS-CoV-MA15-infected mice, we tested the ability of different types of IFNs to sensitise different types of human and mouse cells towards death ligand-induced cell death.

We performed these experiments with and without pre-treatment with type I IFNs (IFN-alpha and/or IFN-beta) and type II IFN (IFN-gamma) to mimic the increased, albeit delayed induction of type I and II IFNs which have been reported to be present in the lungs of patients suffering from SARS coronavirus-induced pathologies and that orchestrate an inflammatory response, yet possibly not being solely responsible for the pathogenesis of the disease (Thompson, Chambers, and Liu 2017; Ackermann et al. 2020; Memish et al. 2020; Fan et al. 2020; Israelow et al. 2020).

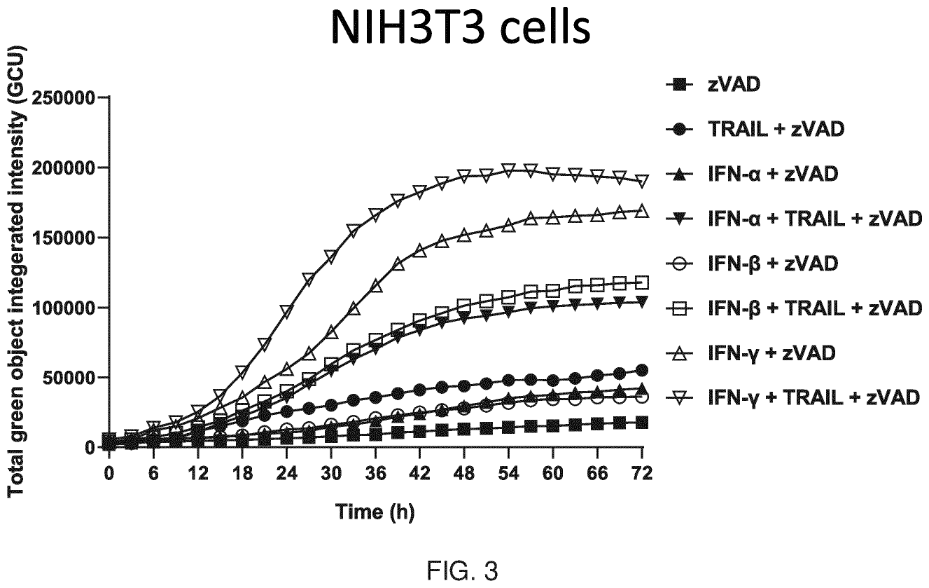

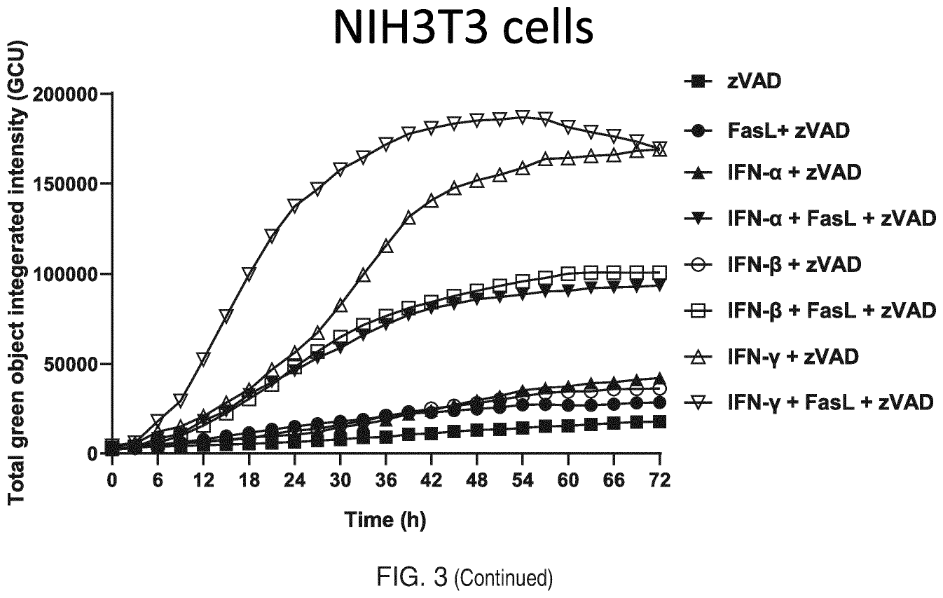

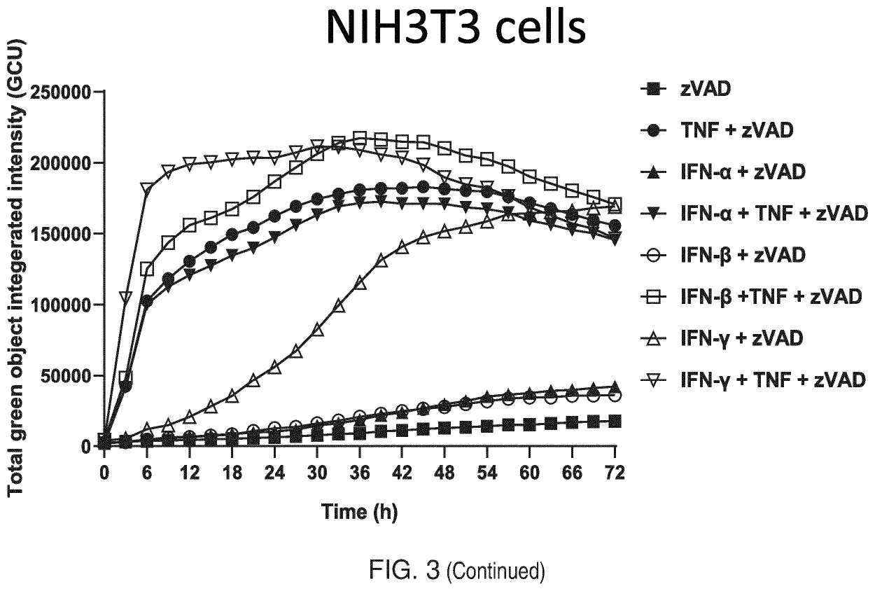

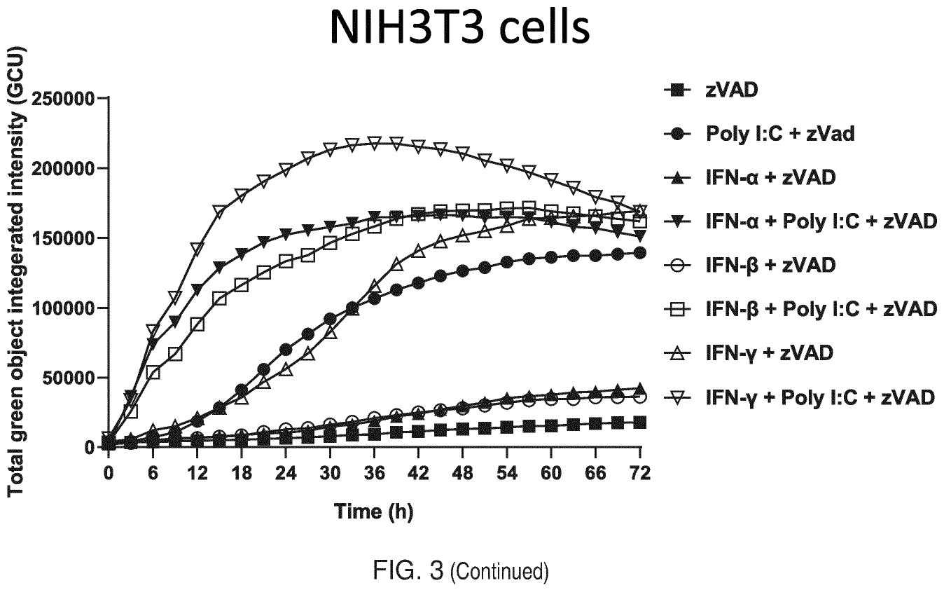

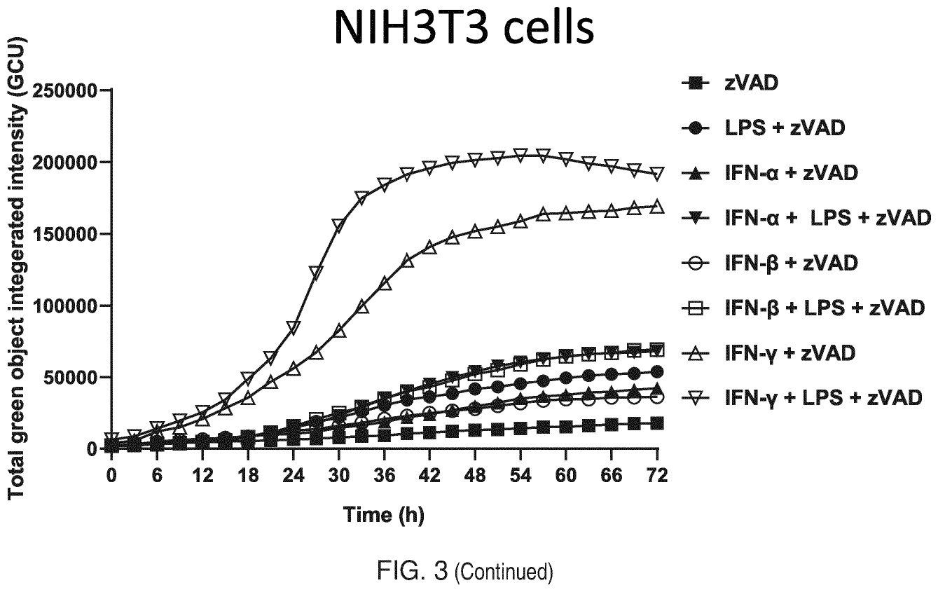

Pretreatment of NIH3T3 mouse fibroblasts with IFN-alpha, IFN-beta or IFN-gamma significantly sensitised them to death induction by TRAIL or FasL in the presence of caspase inhibition (zVAD-fmk, zVAD). Notably, treatment with IFN-gamma alone was sufficient to significantly induce cell death, albeit to a lesser extent than when combined with TRAIL or FasL (FIG. 3).

### Example 4: Effect of Different Types of Interferons on Death Ligand-Induced Cell Death in Human Cells

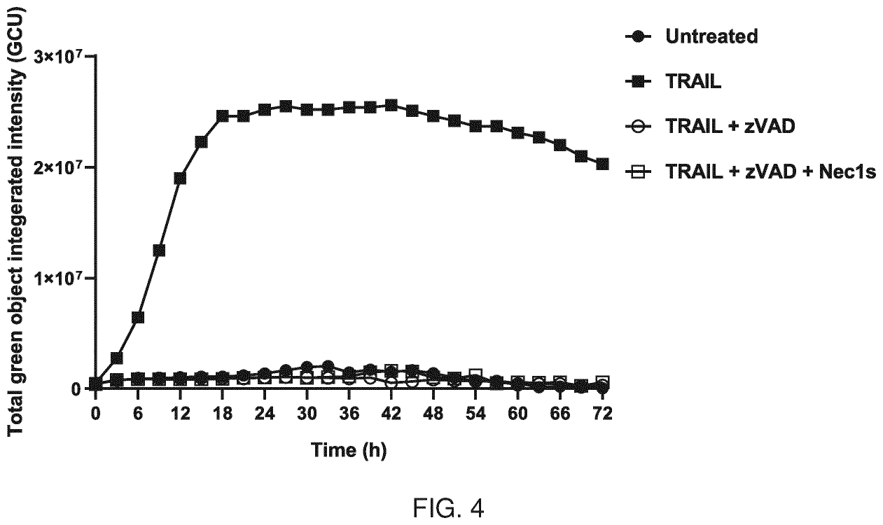

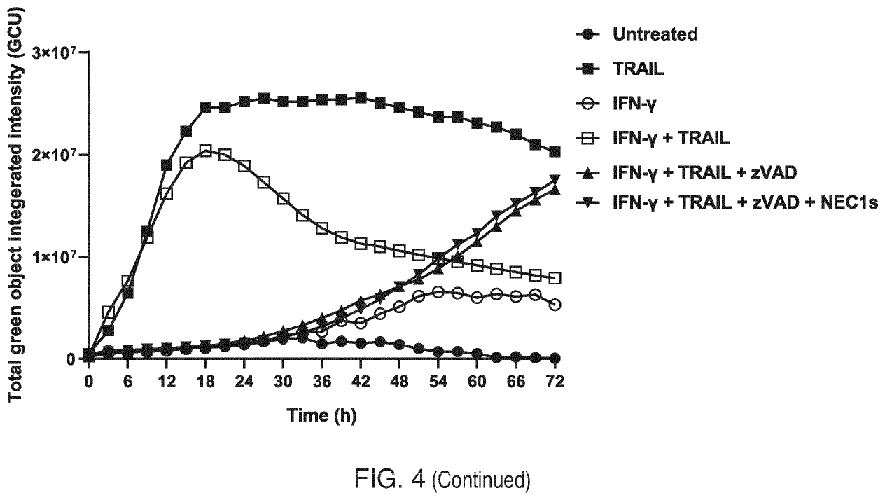

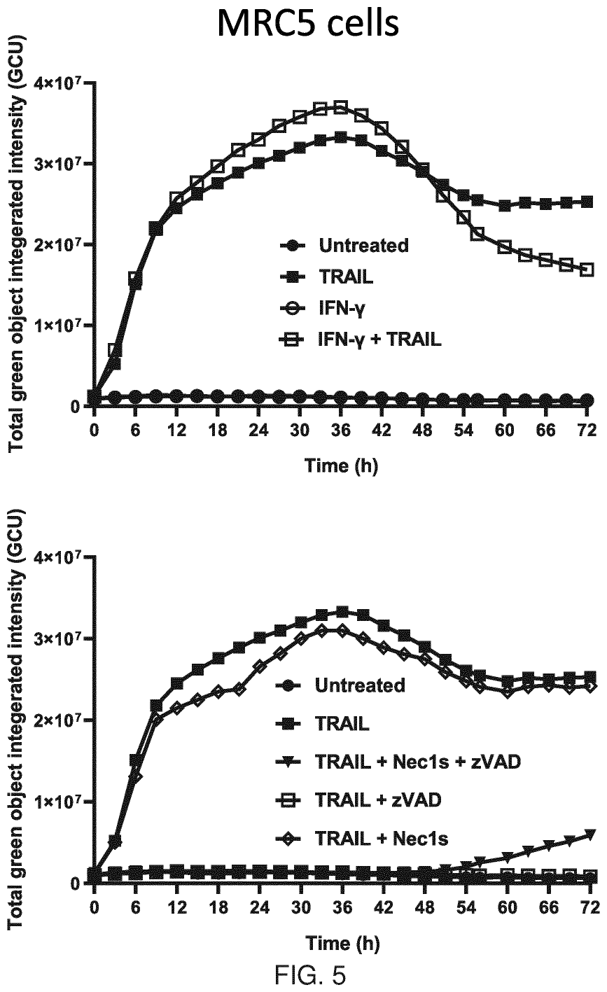

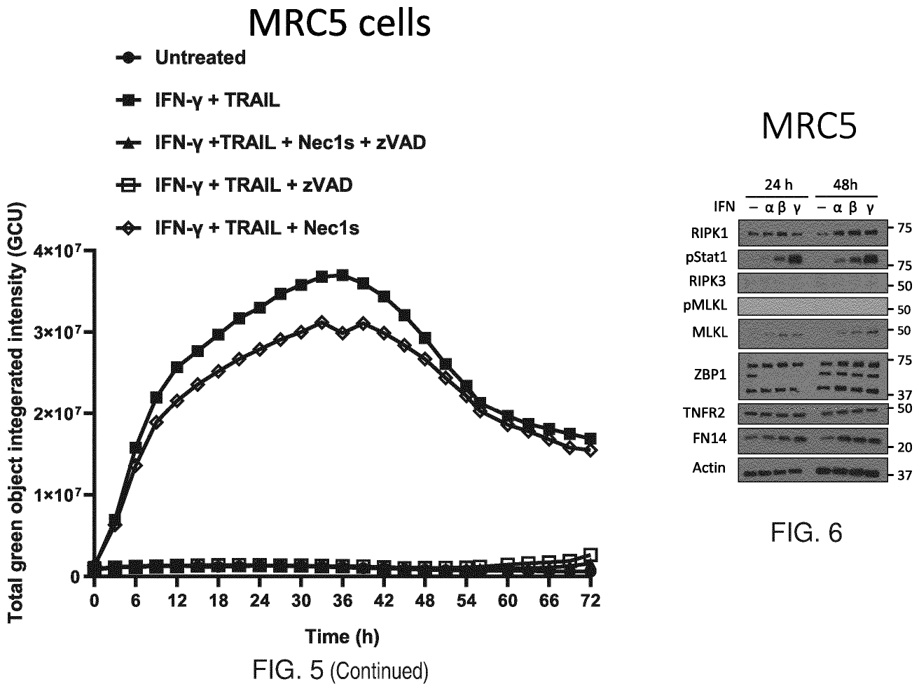

We next treated non-transformed human pancreatic ductal epithelial cells (HPDEC) (FIG. 4) and primary human lung fibroblasts (MRC5 cells) (FIGS. 5, 6). Whilst the choice of primary human lung fibroblast is consistent with those aspects of the present invention concerning the pathology in inflammatory disease caused in the respiratory tract as a consequence of infection by a SARS coronaviruses or other viruses, HPDEC cells were specifically selected for two reasons: (i) ACE2 has been found to be highly expressed in the pancreas, especially by ductal epithelial cells cells (Liu, Long, et al. 2020), and (ii) individuals infected with SARS-CoV-2, including individuals who underwent asymptomatic infection, appear to develop a previously unknown new form of Type I Diabetes (T1D) (Li et al. 2020; Yang et al. 2010; Chee, Ng, and Yeoh 2020; Rubino et al. 2020), thereby expanding the potential usefulness of the therapies provided herein to preventing damage to organs other than the lung, including e.g. the pancreas but, by the use of the pancreas as an example, also to other organs, e.g. liver, kidney, heart and brain.

The results of these experiments show that HPDECs and MRC5 cells are both exquisitely sensitive to TRAIL-induced apoptosis (FIGS. 4, 5). They also reveal that pre-treatment with IFN-gamma did not increase their sensitivity to cell death induction by TRAIL even though in HPDECs, IFN-gamma pre-treatment accelerated TRAIL-induced cell death when compared to treatment with TRAIL alone (FIG. 4). Whereas the observed cell death in MRC5 cells could not be blocked by inhibition of RIPK1 kinase (achieved with the RIPK1 kinase inhibitor Nec-1s), this was completely blocked by inhibition of caspases (achieved with zVAD-fmk) alone (FIG. 5). By contrast, whilst treatment with zVAD, Nec-1s or the combination of zVAD with Nec-1s delayed the death of HPDECs induced by TRAIL in combination with IFN-gamma, it could not prevent it (FIG. 4). These results suggest that the combination of IFN-gamma and TRAIL induces, at least at later times, a type of death in HPDECs that may be triggered by programmed cell death pathways that are independent of caspases or the kinase activity of RIPK1, e.g. ZBP1-mediated necroptosis and/or inflammasome-mediated pyroptosis, and/or by CRS induced by the activation of inflammatory pathways, e.g as a consequence of cGAS/STING signalling (Hopfner and Hornung 2020; Ma et al. 2020). Consequently, specific inhibitors of caspases and/or RIPK1 kinase activity may only exert limited, if any, protection for these cells, whereas inhibition of the death-inducing ligands, e.g. of TRAIL, FasL, TNF and/or LT-alpha, achieved, e.g. as explained above (that is, e.g., with a ligand-inhibiting antibody or with a soluble receptor-Fc protein, e.g. TRAIL-R2-Fc in the case of TRAIL, Fas-Fc (Asunercept) in the case of FasL, anti-TNF (e.g. Remicade or Humira) or TNF-R2-Fc (Etanercept/Enbrel) in the case of TNF, and e.g. Etanercept/Enbrel in the case of TNF and LT-alpha), may prevent the killing of these cells, either alone or when combined further with caspase inhibitors and/or inhibitors of RIPK1, RIPK3 and/or MLKL and/or with inhibitors of cGAS/STING signalling and/or with inhibitors of the inflammasome, e.g. inhibitors of the NLRP3 inflammasome such as, e.g. MAS825, and, thereby, protect these organs from failure, i.e. in the case of the pancreas, prevent diabetes, in the case of the heart prevent chronic heart disease, in the case of the liver prevent chronic liver damage, including, e.g. liver fibrosis, and in the case of the brain, loss of certain brain functions up to dementia. Hence, the therapeutic consequences we can extrapolate from these results extend well beyond the treatment of respiratory virus-induced pathologies of the lung and other organs to acute or chronic failure of and/or damage to the lung and other organs in the afore-mentioned pathologies.

The sensitisation shown in both cells lines is clearly indicative of the potent combination a type II IFN response activation can exert in the presence of a death ligand, especially in the presence of TRAIL. However, the biological differences shown by the two cell death mechanisms, i.e. the finding that the human lung fibroblast cell line MRC5 is sensitised by pre-treatment with type II IFN to a TRAIL-induced cell death which is caspase dependent whereas HPDECs are sensitised by the same pre-treatment to a TRAIL-induced cell death which cannot be fully blocked with caspase and/or RIPK1 kinase inhibitors, highlights the contribution of different cell death modalities to the pathogenesis of IFN-mediated inflammatory diseases. This discovery also proposes that the inhibition of the death ligands that induce the cell death in combination with the inhibition of signalling exerted by IFNs, including type I, type II and/or type III IFNs, may be preferable to death ligand inhibition alone or to the inhibition of cell death induction downstream of death receptor engagement by its cognate ligand, i.e. in the case of TRAIL with TRAIL-R1 and/or TRAIL-R2, by the use of inhibitors of caspases, RIPK1, RIPK3 and/or MLKL. The fact that MRC5 cells and HPDECs originate from different organs and cell lineages implies fundamental differences in cell death mechanisms in these distinct cell types. These include differences among various cell types which can be present in the same organ or tissue but also among different organs and tissues and their corresponding sensitivity to cell death induction by different death ligands in the presence of IFNs, including, but not limited to, including type I, type II and/or type III IFNs. This highlights the importance of considering the combined inhibition of one or more different death ligands and/or cell death modalities, either alone or together with the inhibition of different IFNs and/or the pathways they stimulate (N.B. IFN signalling requires JAK1/2-mediated phosphorylation of STAT1 (Schindler et al. 1992; Müller et al. 1993; Abramovich et al. 1994)), potentially not only to prevent or reduce damage to one tissue, e.g. the lung, but also to prevent or reduce damage to other, additional organs, whether potentially infected by the virus in question or not. Specifically, the results we report here propose that the therapeutic inhibition of TRAIL, of type I and/or type II IFNs or the signalling they exert, of the kinases JAK1/2, i.e. the kinases required for signalling by type I and/or type II IFNs, may all exert a therapeutic effect on their own but that a synergistic therapeutic effect can be expected from the combined inhibition of TRAIL and JAK1/2 or of TRAIL and type I and/or type II IFN signalling, e.g. by antagonistic antibodies to these IFNs or, preferably to the specific receptors they bind to, e.g. IFN-alpha Receptor (IFNAR) for type I IFNs. This can be achieved, e.g., but not limited to, by Anifrolumab, an Anti-Interferon-α Receptor Monoclonal Antibody which is currently in clinical trials for the treatment of SLE (Furie et al. 2017) or by BIIB059, a humanised monoclonal antibody that binds blood DC antigen 2 (BDCA2), a plasmacytoid dendritic cell (pDC)-specific receptor that inhibits the production of type I IFN and other inflammatory mediators (Furie et al. 2019). Because of the afore-mentioned results, such efficacies of these therapies are likely to increase significantly through combination with at least one anti-inflammatory agent.

Interestingly, western blot analysis of MRC5 cells revealed upregulation of key cell death factors following treatment with all IFNs (FIG. 6), particularly factors involved in the induction of necroptosis, including RIPK1 and ZBP1. Similarly, MLKL was strongly upregulated in MRC5 cells, particularly following treatment with IFN-gamma, whilst ZBP1 was upregulated following treatment with all IFNs tested. This suggests that IFN-gamma treatment in combination with TRAIL could involve activation of RIPK1 and of downstream effectors of RIPK1 such as, but not limited to, MLKL, but also of ZBP1, which can activate RIPK3 and MLKL independently of RIPK1. This proposes the utilisation of a combination approach between a TRAIL inhibitor such as, e.g., TRAIL-R2-Fc, and a drug capable of inhibiting the kinase activity of RIPK1 including, but not limited to, the RIPK1 kinase inhibitors GSK2982772, GSK095, DNL747 and DNL758 (Hamilton et al. 2019; Hou et al. 2019; Wang et al. 2019) and/or a drug capable of inhibiting the kinase activity of RIPK3 and/or a drug capable of inhibiting the cell death-promoting activity of MLKL.

### Example 5: Effect of Different Types of Interferons on Death Ligand-Induced Cell Death in Lung Endothelial Cells

To further expand our biological understanding of death ligand-induced cell death in different cellular populations of lung origin, we utilised primary mouse lung endothelial cells. This cellular system is particularly relevant in the context of COVID-19 given that lung endothelial cells normally limit the influx of fluid to the interstitial and alveolar compartments. In addition, normal physiological function of the lung endothelium appears to be disrupted in many patients with severe or critical COVID-19 (Zhou et al. 2020; Xu et al. 2020; Yang et al. 2020), specifically but also more generally, in patients suffering from ARDS (Chambers, Rounds, and Lu 2018; Abadie et al. 2005; Gill, Rohan, and Mehta 2015; Gill et al. 2014; Fujita et al. 1998). Moreover, the interaction with blood-borne cells and vasoactive mediators, renders the lung endothelium capable of sensing and responding to different mechanical, chemical and cellular stimuli. While this makes the endothelial cells able to regulate local inflammation, the disruption of the endothelium can lead from an initial anti-inflammatory response to an activated pro-inflammatory phenotype that propagates and amplifies lung inflammation (Zimmermann et al. 1999) and consequently cell death. In addition, this cell type is of particular interest given the discovery that the level of expression of certain death ligands and the level of presence of certain immune cell types showed significant correlation with the decrease in the presence of different types of endothelial cells from the lungs of mice infected with SARS-CoV-MA15 (FIG. 2), suggestive of these endothelial cells being killed by the death ligands expressed by one or more of these immune cells.

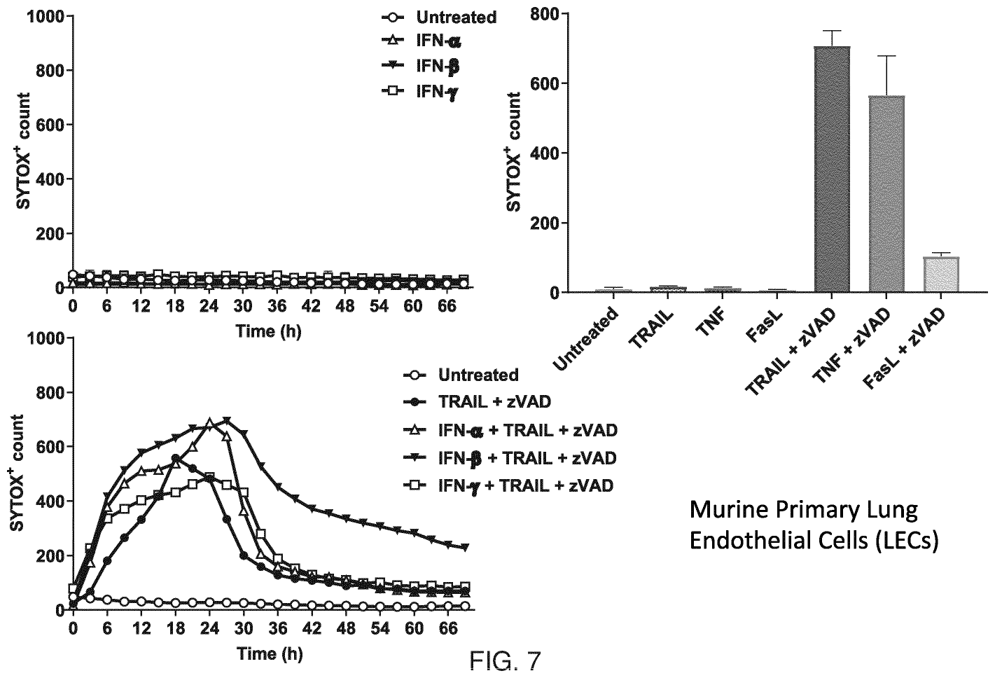

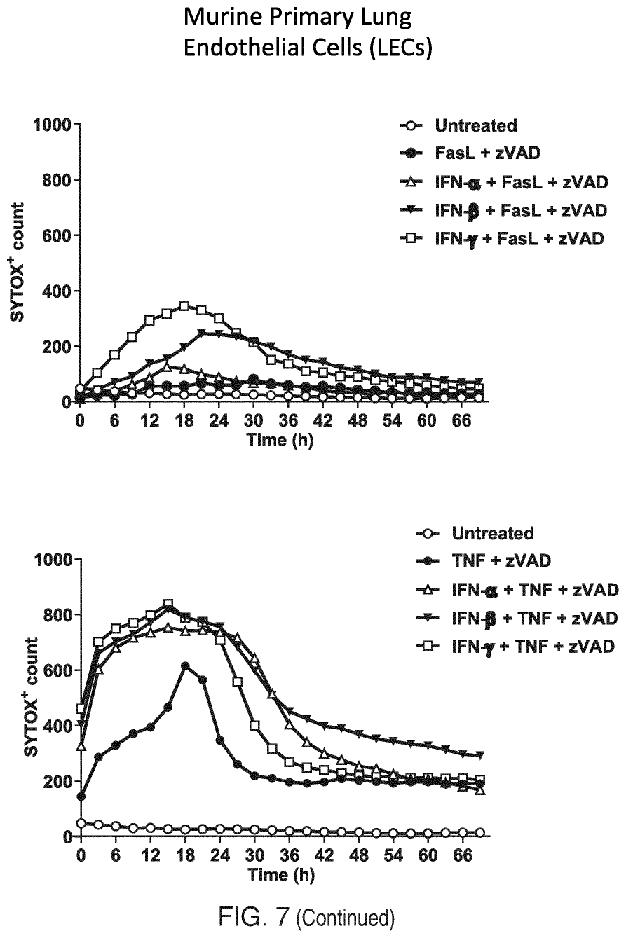

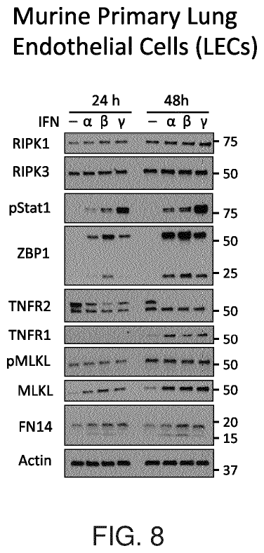

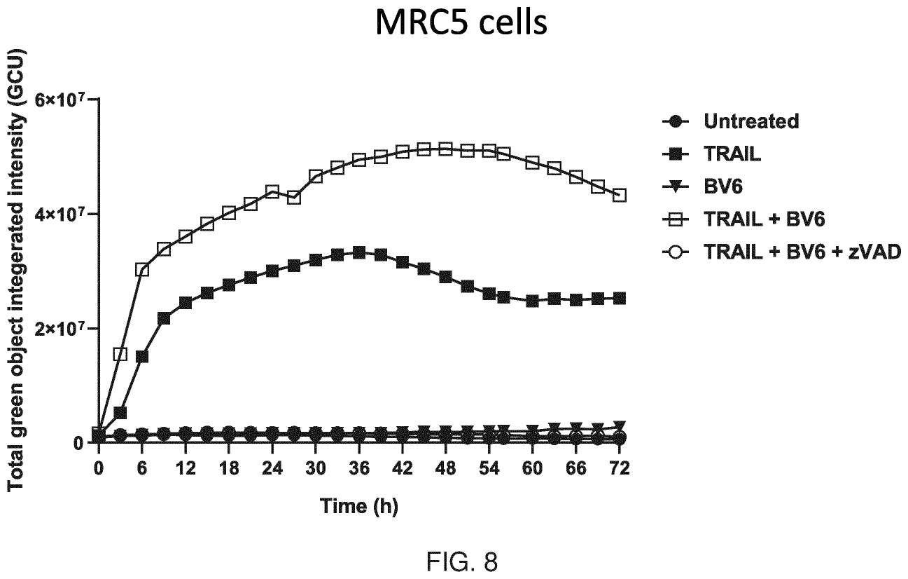

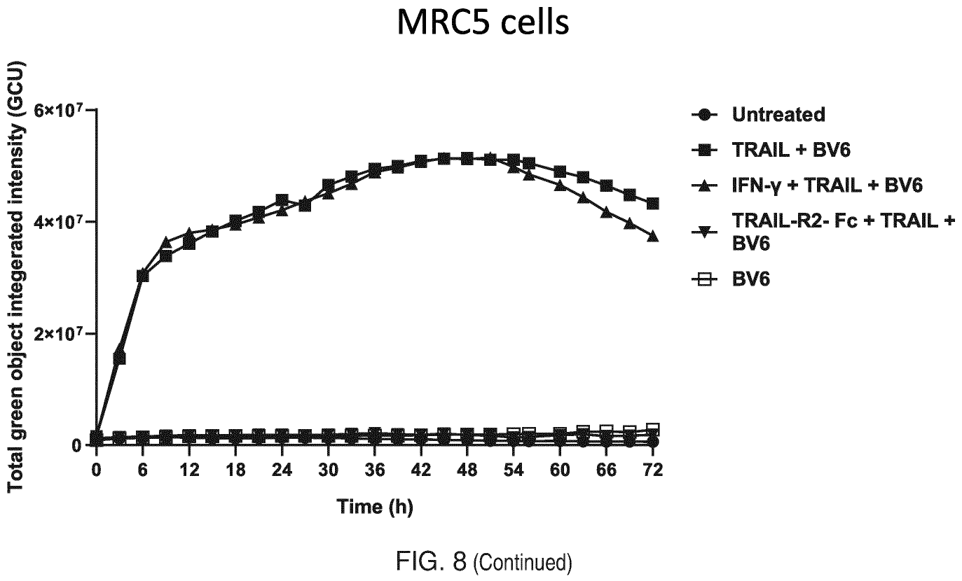

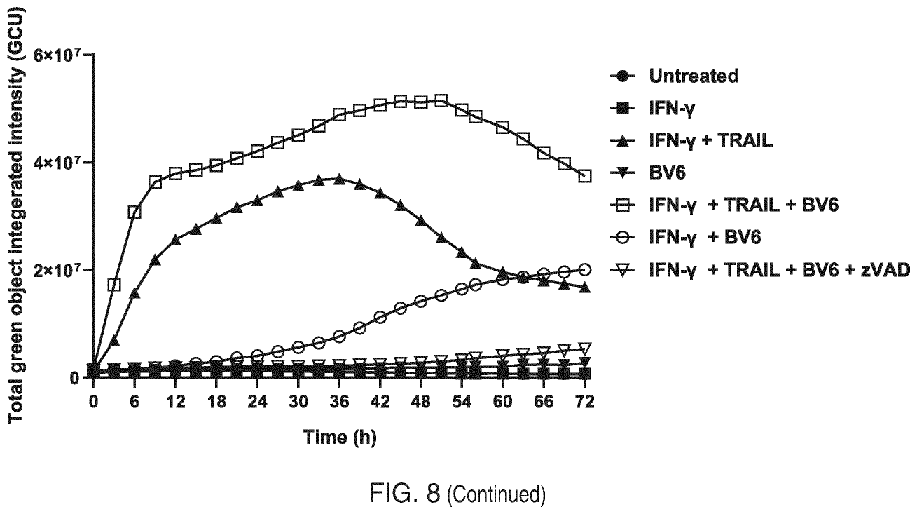

Freshly isolated mouse lung endothelial cells (LECs) were highly sensitive to cell death induction by TRAIL or TNF, yet they were relatively insensitive to treatment with FasL (FIG. 7). Treatment with the different types of IFNs further increased sensitivity to TRAIL or TNF-induced cell death and, importantly, particularly pre-treatment with IFN-gamma also sensitised these cells to FasL-mediated death (FIG. 7). These results imply that lung endothelial cells may be rendered sensitive to a cell-to-cell-induced death in the presence of IFNs, especially of type II IFN (FIG. 7). In all cases the cell death observed in mouse lung endothelial cells required inhibition of caspases by zVAD suggesting a non-apoptotic modality of cell death at play (FIG. 7). Consistent with this, lung endothelial cells upregulated RIPK1, MLKL and both, the short and the long forms of ZBP1 following treatment with the different IFNs, indicating a strong upregulation of the necroptosis pathway components' (FIG. 8). Therefore, this supports the use of a combinatorial approach between necroptosis inhibitors (specifically but not limited to RIPK1 kinase inhibitors) and death ligand blockers.

### Example 6: Effect of Different Types of Interferons on Death Ligand-Induced Cell Death Under Conditions Simulating the Patho-Physiological Conditions as Identified by the Results Shown in Example 2

As mentioned above, the targeted analysis of the mRNA expression data obtained from the lungs of SARS-CoV-MA15-infected mice (Chen et al. 2010; McDermott et al. 2016) revealed that the death ligands TRAIL, FasL, TNF and LT-alpha are all significantly upregulated on various and varying types of cells, particularly immune cells, that are present in the lungs of these mice (FIG. 2A-D). Intriguingly, this was also true for TNFR2 and FN14 which were indeed amongst the most highly upregulated genes in the lungs of infected mice (FIG. 2E, F). Triggering of TNFR2 or FN14 by their respective ligands, TNF and TWEAK, results in non-canonical NF-kB activation (Saitoh et al. 2003; Roos et al. 2010; Rauert et al. 2010; Munroe and Bishop 2004). This signalling involves the proteasomal degradation of the cellular inhibitor of apoptosis proteins 1 and 2 (clAP1 and clAP2) (Saitoh et al. 2003; Rauert et al. 2010; Zarnegar et al. 2008). Intriguingly, degradation of clAP1 and clAP2 is known to sensitise cells to death induction by TNF, TRAIL, FasL, poly-I:C and LPS (Guicciardi et al. 2011; Tamm et al. 1998; Feoktistova et al. 2011; Varfolomeev et al. 2008; Tuthill et al. 2015). Ligands for TLR3 can be derived from both, the viral RNA itself or from endogenous RNA (Zinngrebe et al. 2016; Alexopoulou et al. 2001; Brentano et al. 2005; Kariko et al. 2004; Jiao et al. 2020) and can trigger cell death via this receptor when the normal physiological balance of factors that determine the signalling output by TLR3 is disturbed, e.g. by TNFR2- or FN14-mediated degradation of clAP1/2 (Saitoh et al. 2003; Roos et al. 2010; Rauert et al. 2010; Munroe and Bishop 2004), which can be pharmacologically mimicked, e.g., by the use of so-called IAP antagonists (also referred to as SMAC mimetics) which deplete cells of clAP1 and clAP2 and, depending on which type of IAP antagonist is used, also neutralise XIAP, and, consequently, tip the balance of death receptor-induced signalling, including of signalling induced by TLR3, towards increased cell death which can be causative for inflammation and inflammation-related pathologies, including pathologies induced by respiratory viruses such as influenza A virus (Zinngrebe et al. 2016).

Considering the observed upregulation of FN14 induced by all IFNs including IFN-gamma in MRC5 cells (FIG. 6), we treated these cells with a bivalent IAP antagonist (BV6) which induces the degradation of clAP1 and clAP2 (Saitoh et al. 2003; Rauert et al. 2010; Zarnegar et al. 2008). Whereas treatment with BV6 alone did not sensitise these cells, the combination of BV6 with TRAIL or with IFN-gamma alone or with IFN-gamma and TRAIL revealed enhanced toxicity (FIG. 9). Treatment with TRAIL-R2-Fc could completely block the cell death observed by the combination of BV6 with TRAIL (FIG. 9). All of the cell death induced in these cells by the different combinations was entirely apoptotic as it was completely inhibited by z-VAD (FIG. 9).

### Example 7: Treatments Based on TRAIL Inhibition

As explained above, the present inventors have noted that soluble TRAIL is found at significantly increased levels in the bronchoalveolar lavage (BAL) fluid of patients with ARDS (Lee et al. 2008; Bem et al. 2010). Furthermore, it has been reported that activation of the TRAIL-TRAIL-R pathway induces alveolar epithelial cell death and consequent lung injury (Bem et al. 2010; Hogner et al. 2013; Katalan et al. 2017; Rong et al. 2018; Girkin et al. 2017).

Based on the disclosure herein it is plausible that inhibition of cell death induced by one or more death ligands can prevent a significant portion of the pathological cell death that is induced in COVID-19 patients and, whilst this alone may be capable of providing therapeutic benefit to certain patients, on the basis of our above-described disclosure, it is believed that the therapeutic benefit can be substantially increased when the cell death-inhibitory therapy is combined with an anti-inflammatory therapy.

One of the present inventors, together with other authors, previously showed that TRAIL blockade reduces virus production following infection of cells with IAV and other reported similar findings following Rhinovirus 1B (R1B) infection (Wurzer et al. 2004; Girkin et al. 2017). In addition, genetic deletion of TRAIL was shown to ameliorate R1B-induced lung injury (Girkin et al. 2017). Preventative pharmacologic TRAIL blockade or genetic deletion of TRAIL preserves alveolar fluid clearance, reduces alveolar epithelial cell death and, consequently, increases survival following IAV infection in mice (Herold et al. 2008; Davidson et al. 2014; Peteranderl et al. 2016).

Finally, some of the present inventors, together with other authors, previously showed that TRAIL signalling induces the expression of CCL2 which results in the recruitment of monocytic cells (Hartwig et al. 2017). Inflammatory monocyte macrophages (IMMs) were shown to be the main culprits of the inflammatory disease which is triggered by a delayed but overshooting type I IFN response in a SARS-CoV-MA15 model (Channappanavar et al. 2016). Importantly, these IMMs are recruited to the lungs in a CCL2-dependent manner (Matsushima et al. 1989; Gendelman et al. 2009).

Therefore, apart from its function as a factor that kills cells, TRAIL could also contribute to, or even be responsible for, the recruitment of IMMs to the lungs of infected individuals. In line with the results shown in FIG. 2, these IMMs could kill, or contribute to the killing of, e.g. alveolar type I and/or type II epithelial cells, alveolar and/or bronchial endothelial cells, pericytes, vascular and/or airway smooth muscle cells, fibroblasts, myofibroblast, lipofibroblasts, club cells, or other cells in the lungs and/or other organs of patients with acute organ damage, including ARDS and including when caused, e.g., by viral infection, including, e.g., by R1B or related viruses, IAV, IBV, SARS-CoV(-1), MERS-COV, SARS-CoV-2 or any other virus or other infectious or non-infectious agent.

### Example 8: Novel Treatments Based on Combinations of Therapeutics, Optionally Including Anti-Inflammatory Agents

On the basis of these results, when considered in combination with the results shown in FIG. 1, one can conclude that whilst inhibiting TRAIL, FasL, TNF and/or LT-alpha, e.g. with ligand-inhibiting antibodies and/or with soluble receptor-Fc proteins, e.g. TRAIL-R2-Fc in the case of TRAIL, Fas-Fc (Asunercept) in the case of FasL, anti-TNF (e.g. Remicade or Humira) or TNF-R2-Fc (Etanercept/Enbrel) in the case of TNF, and e.g. Etanercept/Enbrel in the case of TNF and LT-alpha, may suffice to inhibit the diminution of different cell types in the lungs of SARS-CoV-MA15-infected mice, most likely by preventing the induction of their untoward death, it is highly likely that the combination with at least one anti-inflammatory drug will result in a substantial increase in the efficacy of these treatments. This rationale likely also applies to the treatment of patients infected by a SARS coronavirus such as, e.g., SARS-CoV(-1), MERS-COV, SARS-CoV-2 and/or, importantly, any SARS coronavirus or also other viruses, including but not limited to influenza viruses, rhinoviruses or reoviruses or other viruses, including coronaviruses and influenza viruses which may emerge as viruses capable of infecting humans in the future.

On the basis of these analyses, we expect that (an) agent(s) that inhibit(s) TRAIL, FasL, TNF and/or LT-alpha and/or the cell death and/or other signals they induce, can achieve a significant therapeutic effect in certain COVID-19 patients, especially in patients in whom the inflammation is not yet very severe and/or advanced.

To maximise the achievable therapeutic effect in other patients suffering from COVID-19, for example in patients in whom the inflammation is more severe and/or advanced, it is likely that such (an) agent(s) need(s) to be combined with one or more anti-inflammatory drug.

At the same time, these results imply that the inhibition of TRAIL, FasL, TNF and/or LT-alpha alone may be sufficient to achieve a significant therapeutic effect in certain patients suffering from ARDS induced by other viruses, bacteria or fungi or when ARDS is not caused by an infectious agent, e.g. by reflux or by other means, whilst combination with other active agents, as explained herein, preferably with at least one anti-inflammatory agent, may be required to achieve a more pronounced therapeutic effect in other patients.

Another important aspect to consider is that these data, together with the reported upregulation of TRAIL, FasL, TNF, and LT-alpha in the lungs of infected mice, do not only support a rationale whereby the activation of their respective receptors leads to cell death-induced inflammation (as shown with the data provided above). The ligand-mediated activation of these receptors also induces the transcriptional activation of genes which, independently of the capacity of these receptor-ligand systems to induce cell death, will contribute to inflammation and to recruitment, infiltration and activation of different types of immune cells which will shape the cellular immune microenvironment and thereby contribute to the exacerbation of inflammation and prevention of immunity. Importantly, for TRAIL we previously reported that the stimulation of epithelial cells which are not killed by TRAIL leads to the production of various cytokines, including CCL2, which we found to be responsible for triggering an immune microenvironment which prevented immune recognition of lung cancer and, at the same time, promoted lung cancer growth through the recruitment of CCR2-expressing myeloid cells into the tumour immune microenvironment (Hartwig et al. 2017) which are known to be immuno-suppressive cells (Lesokhin et al. 2012).

Intriguingly, CCL2 has been implicated in the recruitment of harmful immune cells into the lungs of patients suffering from COVID-19 (Blanco-Melo et al. 2020).

Thus, inhibiting TRAIL may exert its therapeutic efficacy not only through inhibition of TRAIL-mediated cell death but also through inhibition of the TRAIL-dependent production of cytokines, e.g., but not limited to, the production of CCL2. In the absence of a TRAIL inhibitor such cytokines create an immune microenvironment in which the development of an adaptive immune response is actively interfered with, thereby preventing immune recognition and development of an effective adaptive immune response to SARS-CoV-2.

We therefore propose that, apart from limiting tissue damage in COVID-19 patients, therapeutic inhibition of TRAIL may also enable more effective immune recognition and more effective and accelerated development of immunity to, and consequently elimination of, SARS-CoV-2. Since FasL, TNF and LT-alpha have also previously been shown to be capable of inducing non-cell-death-related pro-tumourigenic and/or immuno-suppressive signalling (Chavez-Galan et al. 2017; Salomon et al. 2018; Leclerc et al. 2016; Suganuma et al. 1999; Moore et al. 1999; Pikarsky et al. 2004; Popivanova et al. 2008; Or et al. 2010; Charles et al. 2009; Hehlgans et al. 2002; Haybaeck et al. 2009; Ammirante et al. 2010), similar considerations can be made for inhibitors of FasL, TNF and/or LT-alpha.

In summary, together with the further insights and analysis described herein, there is a scientifically sound rationale for therapeutic intervention by inhibition of the death ligands TRAIL, FasL, TNF and/or LT-alpha, and/or the activities resulting from these interactions, with the intention of providing an effective treatment for COVID-19 patients by preventing cell death in the lungs of these patients and the consequent cytokine release syndrome (CRS) and inflammation which causes ARDS. Similar considerations apply to the interactions of dsRNA with TLR3, of LPS with TLR4, of TWEAK with FN14 and of TL1A with DR3.

Given our recent observation of the highly synergistic activity of inhibiting pro-inflammatory cell death and concomitantly inhibiting inflammation, particularly in a pathological situation in which the inflammation is already ongoing (FIG. 1), the therapeutic effect achieved thereby will likely increase substantially when such a therapy is combined with an anti-inflammatory treatment.

### Example 9: In-Vivo Confirmation of Example Treatments of the Invention

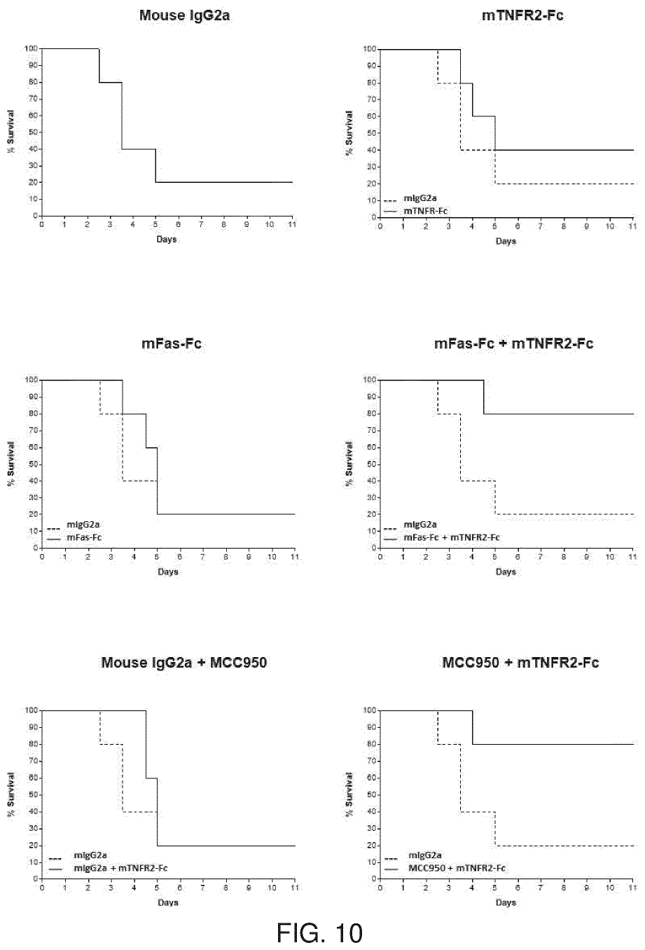

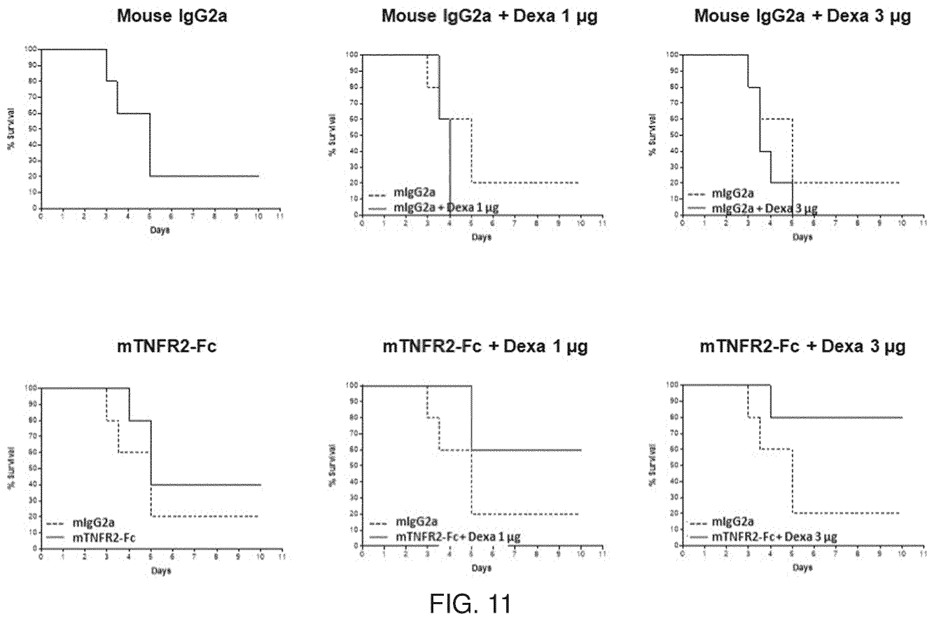

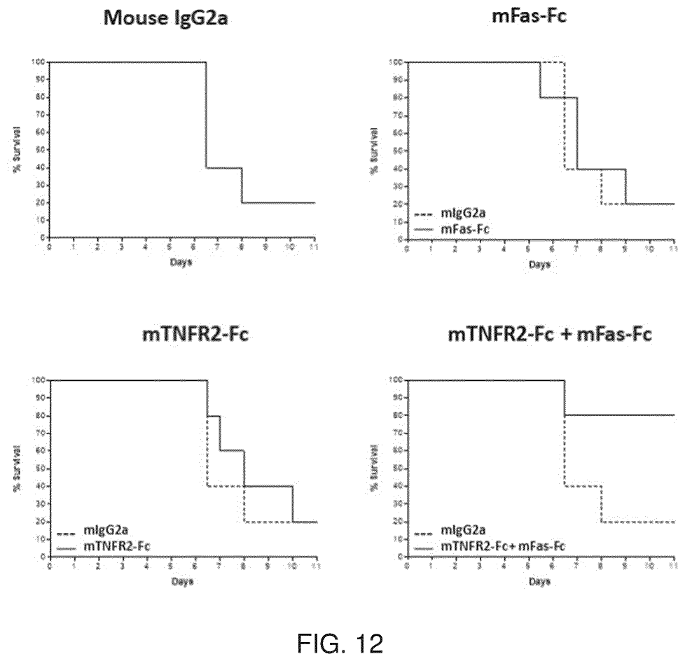

FIGS. 10-12 clearly confirm the benefit of combining inhibition or neutralisation of different death ligands with each other or with anti-inflammatory agents in the treatment of diseases such as ARDS. In each case, combinations according to the invention provided substantial improvements in survival in SARS coronavirus infection-induced mouse models of ARDS, compared to the agents when administered alone.

### REFERENCES

Abadie, Y., F. Bregeon, L. Papazian, F. Lange, B. Chailley-Heu, P. Thomas, P. Duvaldestin, S. Adnot, B. Maitre, and C. Delclaux. 2005. ‘Decreased VEGF concentration in lung tissue and vascular injury during ARDS’, Eur Respir J, 25:139-46.
Abramovich, C., L. M. Shulman, E. Ratovitski, S. Harroch, M. Tovey, P. Eid, and M. Revel. 1994. ‘Differential tyrosine phosphorylation of the IFNAR chain of the type I interferon receptor and of an associated surface protein in response to IFN-alpha and IFN-beta’, Embo J, 13:5871-7.
Ackermann, M., S. E. Verleden, M. Kuehnel, A. Haverich, T. Welte, F. Laenger, A. Vanstapel, C. Werlein, H. Stark, A. Tzankov, W. W. Li, V. W. Li, S. J. Mentzer, and D. Jonigk. 2020. ‘Pulmonary Vascular Endothelialitis, Thrombosis, and Angiogenesis in Covid-19’, N Engl J Med, 383:120-28.
Albertine, K. H., M. F. Soulier, Z. Wang, A. Ishizaka, S. Hashimoto, G. A. Zimmerman, M. A. Matthay, and L. B. Ware. 2002. ‘Fas and fas ligand are up-regulated in pulmonary edema fluid and lung tissue of patients with acute lung injury and the acute respiratory distress syndrome’, Am J Pathol, 161:1783-96.
Alexopoulou, L., A. C. Holt, R. Medzhitov, and R. A. Flavell. 2001. ‘Recognition of double-stranded RNA and activation of NF-kappaB by Toll-like receptor 3’, Nature, 413:732-8.
Alhazzani, Waleed, Morten Hylander Møller, Yaseen M. Arabi, Mark Loeb, Michelle Ng Gong, Eddy Fan, Simon Oczkowski, Mitchell M. Levy, Lennie Derde, Amy Dzierba, Bin Du, Michael Aboodi, Hannah Wunsch, Maurizio Cecconi, Younsuck Koh, Daniel S. Chertow, Kathryn Maitland, Fayez Alshamsi, Emilie Belley-Cote, Massimiliano Greco, Matthew Laundy, Jill S. Morgan, Jozef Kesecioglu, Allison McGeer, Leonard Mermel, Manoj J. Mammen, Paul E. Alexander, Amy Arrington, John E. Centofanti, Giuseppe Citerio, Bandar Baw, Ziad A. Memish, Naomi Hammond, Frederick G. Hayden, Laura Evans, and Andrew Rhodes. 2020. ‘Surviving Sepsis Campaign: guidelines on the management of critically ill adults with Coronavirus Disease 2019 (COVID-19)’, Intensive care medicine, 46:854-87.
Ammirante, Massimo, Jun-Li Luo, Sergei Grivennikov, Sergei Nedospasov, and Michael Karin. 2010. ‘B-cell-derived lymphotoxin promotes castration-resistant prostate cancer’, Nature, 464:302-05.
Arends, J. E., A. I. M. Hoepelman, N. M. Nanlohy, F. J. P. Hoppener, K. R. Hirsch, J. G. Park, and D. van Baarle. 2011. ‘Low doses of the novel caspase-inhibitor GS-9450 leads to lower caspase-3 and -8 expression on peripheral CD4+ and CD8+ T-cells’, Apoptosis: an international journal on programmed cell death, 16:959-66.
Atreya, R., J. Mudter, S. Finotto, J. Müllberg, T. Jostock, S. Wirtz, M. Schütz, B. Bartsch, M. Holtmann, C. Becker, D. Strand, J. Czaja, J. F. Schlaak, H. A. Lehr, F. Autschbach, G. Schürmann, N. Nishimoto, K. Yoshizaki, H. Ito, T. Kishimoto, P. R. Galle, S. Rose-John, and M. F. Neurath. 2000. ‘Blockade of interleukin 6 trans signaling suppresses T-cell resistance against apoptosis in chronic intestinal inflammation: evidence in crohn disease and experimental colitis in vivo’, Nat Med, 6:583-8.
Badley, A. D., D. H. Dockrell, A. Algeciras, S. Ziesmer, A. Landay, M. M. Lederman, E. Connick, H. Kessler, D. Kuritzkes, D. H. Lynch, P. Roche, H. Yagita, and C. V. Paya. 1998. ‘In vivo analysis of Fas/FasL interactions in HIV-infected patients’, The Journal of clinical investigation, 102:79-87.
Barkhausen, Tanja, Thomas Tschernig, Philip Rosenstiel, Martijn van Griensven, Ralf-Peter Vonberg, Martina Dorsch, Annika Mueller-Heine, Athena Chalaris, Jürgen Scheller, and Stefan Rose-John. 2011. ‘Selective blockade of interleukin-6 trans-signaling improves survival in a murine polymicrobial sepsis model’, Read Online: Critical Care Medicine|Society of Critical Care Medicine, 39:1407-13.
Bauer, Torsten T, Santiago Ewig, Arne C Rodloff, and Eckhard E Müller. 2006. ‘Acute respiratory distress syndrome and pneumonia: a comprehensive review of clinical data’, Clinical Infectious Diseases, 43:748-56.
Becker, C., M. C. Fantini, C. Schramm, H. A. Lehr, S. Wirtz, A. Nikolaev, J. Burg, S. Strand, R. Kiesslich, S. Huber, H. Ito, N. Nishimoto, K. Yoshizaki, T. Kishimoto, P. R. Galle, M. Blessing, S. Rose-John, and M. F. Neurath. 2004. ‘TGF-beta suppresses tumor progression in colon cancer by inhibition of IL-6 trans-signaling’, Immunity, 21:491-501.
Bem, R. A., A. P. Bos, R. M. Wosten-van Asperen, M. Bruijn, R. Lutter, M. R. Sprick, and J. B. van Woensel. 2010. ‘Potential role of soluble TRAIL in epithelial injury in children with severe RSV infection’, Am J Respir Cell Mol Biol, 42:697-705.
Bhatraju, P. K., B. J. Ghassemieh, M. Nichols, R. Kim, K. R. Jerome, A. K. Nalla, A. L. Greninger, S. Pipavath, M. M. Wurfel, L. Evans, P. A. Kritek, T. E. West, A. Luks, A. Gerbino, C. R. Dale, J. D. Goldman, S. O'Mahony, and C. Mikacenic. 2020. ‘Covid-19 in Critically Ill Patients in the Seattle Region-Case Series’, N Engl J Med, 30.
Blanco-Melo, Daniel, Benjamin E. Nilsson-Payant, Wen-Chun Liu, Skyler Uhl, Daisy Hoagland, Rasmus Møller, Tristan X. Jordan, Kohei Oishi, Maryline Panis, David Sachs, Taia T. Wang, Robert E. Schwartz, Jean K. Lim, Randy A. Albrecht, and Benjamin R. tenOever. 2020. ‘Imbalanced Host Response to SARS-CoV-2 Drives Development of COVID-19’, Cell, 181:1036-45.e9.
Boch, T., T. Luft, G. Metzgeroth, M. Mossner, J. C. Jann, D. Nowak, F. Meir, C. Schumann, J. Klemmer, S. Brendel, H. Fricke, C. Kunz, C. Weiss, W. K. Hofmann, and F. Nolte. 2018. ‘Safety and efficacy of the CD95-ligand inhibitor asunercept in transfusion-dependent patients with low and intermediate risk MDS’, Leuk Res, 68:62-69.
Brentano, F., O. Schorr, R. E. Gay, S. Gay, and D. Kyburz. 2005. ‘RNA released from necrotic synovial fluid cells activates rheumatoid arthritis synovial fibroblasts via Toll-like receptor 3’, Arthritis Rheum, 52:2656-65.
Chambers, Eboni, Sharon Rounds, and Qing Lu. 2018. ‘Pulmonary Endothelial Cell Apoptosis in Emphysema and Acute Lung Injury’, Advances in anatomy, embryology, and cell biology, 228:63-86.
Channappanavar, R., A. R. Fehr, R. Vijay, M. Mack, J. Zhao, D. K. Meyerholz, and S. Perlman. 2016. ‘Dysregulated Type I Interferon and Inflammatory Monocyte-Macrophage Responses Cause Lethal Pneumonia in SARS-CoV-Infected Mice’, Cell Host Microbe, 19:181-93.
Charles, K. A., H. Kulbe, R. Soper, M. Escorcio-Correia, T. Lawrence, A. Schultheis, P. Chakravarty, R. G. Thompson, G. Kollias, J. F. Smyth, F. R. Balkwill, and T. Hagemann. 2009. ‘The tumor-promoting actions of TNF-alpha involve TNFR1 and IL-17 in ovarian cancer in mice and humans’, The Journal of clinical investigation, 119:3011-23.
Chavez-Galan, Leslie, Dominique Vesin, Husnu Uysal, Guillaume Blaser, Mahdia Benkhoucha, Bernhard Ryffel, Valérie F. J. Quesniaux, and Irene Garcia. 2017. ‘Transmembrane Tumor Necrosis Factor Controls Myeloid-Derived Suppressor Cell Activity via TNF Receptor 2 and Protects from Excessive Inflammation during BCG-Induced Pleurisy’, Frontiers in Immunology, 8.
Chawla-Sarkar, M., D. W. Leaman, and E. C. Borden. 2001. ‘Preferential induction of apoptosis by interferon (IFN)-beta compared with IFN-alpha2: correlation with TRAIL/Apo2L induction in melanoma cell lines’, Clin Cancer Res, 7:1821-31.
Chee, Y. J., S. J. H. Ng, and E. Yeoh. 2020. ‘Diabetic ketoacidosis precipitated by Covid-19 in a patient with newly diagnosed diabetes mellitus’, Diabetes Res Clin Pract.
Cheng, Kui, Xiaohui Wang, and Hang Yin. 2011. ‘Small-molecule inhibitors of the TLR3/dsRNA complex’, Journal of the American Chemical Society, 133:3764-67.
Chiang, Eugene Y, Ganesh A Kolumam, Xin Yu, Michelle Francesco, Sinisa Ivelja, Ivan Peng, Peter Gribling, Jean Shu, Wyne P Lee, and Canio J Refino. 2009. ‘Targeted depletion of lymphotoxin-α-expressing TH 1 and TH 17 cells inhibits autoimmune disease’, Nature medicine, 15:766-73.
Chow, W. A., J. J. Fang, and J. K. Yee. 2000. ‘The IFN regulatory factor family participates in regulation of Fas ligand gene expression in T cells’, J Immunol, 164:3512-8.
Croft, M., and R. M. Siegel. 2017. ‘Beyond TNF: TNF superfamily cytokines as targets for the treatment of rheumatic diseases’, Nature reviews. Rheumatology, 13:217-33.
Czock, David, Frieder Keller, Franz Maximilian Rasche, and Ulla Haussler. 2005. ‘Pharmacokinetics and pharmacodynamics of systemically administered glucocorticoids’, Clinical pharmacokinetics, 44:61-98.
Dashkevich, A., A. Raissadati, S. O. Syrjälä, G. Zarkada, M. A. Keränen, R. Tuuminen, R. Krebs, A. Anisimov, M. Jeltsch, V. M. Leppänen, K. Alitalo, A. I. Nykänen, and K. B. Lemström. 2016. ‘Ischemia-Reperfusion Injury Enhances Lymphatic Endothelial VEGFR3 and Rejection in Cardiac Allografts’, Am J Transplant, 16:1160-72.
Davidson, S., S. Crotta, T. M. McCabe, and A. Wack. 2014. ‘Pathogenic potential of interferon αβ in acute influenza infection’, Nat Commun, 5.
Davila, M. L., I. Riviere, X. Wang, S. Bartido, J. Park, K. Curran, S. S. Chung, J. Stefanski, O. Borquez-Ojeda, M. Olszewska, J. Qu, T. Wasielewska, Q. He, M. Fink, H. Shinglot, M. Youssif, M. Satter, Y. Wang, J. Hosey, H. Quintanilla, E. Halton, Y. Bernal, D. C. Bouhassira, M. E. Arcila, M. Gonen, G. J. Roboz, P. Maslak, D. Douer, M. G. Frattini, S. Giralt, M. Sadelain, and R. Brentjens. 2014. ‘Efficacy and toxicity management of 19-28z CAR T cell therapy in B cell acute lymphoblastic leukemia’, Sci Transl Med, 6:3008226.
DeDiego, M. L., J. L. Nieto-Torres, J. M. Jimenez-Guardeno, J. A. Regla-Nava, C. Castano-Rodriguez, R. Fernandez-Delgado, F. Usera, and L. Enjuanes. 2014. ‘Coronavirus virulence genes with main focus on SARS-CoV envelope gene’, Virus Res, 194:124-37.
Dhein, J., H. Walczak, C. Baumler, K. M. Debatin, and P. H. Krammer. 1995. ‘Autocrine T-cell suicide mediated by APO-1/(Fas/CD95)’, Nature, 373:438-41.
Ellis, G. T., S. Davidson, S. Crotta, N. Branzk, V. Papayannopoulos, and A. Wack. 2015. ‘TRAIL+ monocytes and monocyte-related cells cause lung damage and thereby increase susceptibility to influenza-Streptococcus pneumoniae coinfection’, EMBO Rep, 16:1203-18.
Fan, E., J. R. Beitler, L. Brochard, C. S. Calfee, N. D. Ferguson, A. S. Slutsky, and D. Brodie. 2020. ‘COVID-19-associated acute respiratory distress syndrome: is a different approach to management warranted?’, Lancet Respir Med, 8:816-21.
Fauster, A., M. Rebsamen, K. V. Huber, J. W. Bigenzahn, A. Stukalov, C. H. Lardeau, S. Scorzoni, M. Bruckner, M. Gridling, K. Parapatics, J. Colinge, K. L. Bennett, S. Kubicek, S. Krautwald, A. Linkermann, and G. Superti-Furga. 2015. ‘A cellular screen identifies ponatinib and pazopanib as inhibitors of necroptosis’, Cell Death Dis, 6:130.
Fava, Roy A, Susan M Kennedy, Sheryl G Wood, Anne I Bolstad, Jadwiga Bienkowska, Adrian Papandile, John A Kelly, Clio P Mavragani, Margaret Gatumu, and Kathrine Skarstein. 2011. ‘Lymphotoxin-beta receptor blockade reduces CXCL13 in lacrimal glands and improves corneal integrity in the NOD model of Sjögren's syndrome’, Arthritis Research & Therapy, 13: R182.
Feoktistova, M., P. Geserick, B. Kellert, D. P. Dimitrova, C. Langlais, M. Hupe, K. Cain, M. MacFarlane, G. Hacker, and M. Leverkus. 2011. ‘clAPs block Ripoptosome formation, a RIP1/caspase-8 containing intracellular cell death complex differentially regulated by cFLIP isoforms’, Mol Cell, 43:449-63.
Freeman, Tracey L., and Talia H. Swartz. 2020. ‘Targeting the NLRP3 Inflammasome in Severe COVID-19’, Frontiers in Immunology, 11.
Fujikura, D., S. Chiba, D. Muramatsu, M. Kazumata, Y. Nakayama, T. Kawai, S. Akira, H. Kida, and T. Miyazaki. 2013. ‘Type-I interferon is critical for FasL expression on lung cells to determine the severity of influenza’, PloS one, 8:8.
Fujita, M., K. Kuwano, R. Kunitake, N. Hagimoto, H. Miyazaki, Y. Kaneko, M. Kawasaki, T. Maeyama, and N. Hara. 1998. ‘Endothelial cell apoptosis in lipopolysaccharide-induced lung injury in mice’, Int Arch Allergy Immunol, 117:202-8.
Furie, R., M. Khamashta, J. T. Merrill, V. P. Werth, K. Kalunian, P. Brohawn, G. G. Illei, J. Drappa, L. Wang, S. Yoo, and C. D. Study Investigators. 2017. ‘Anifrolumab, an Anti-Interferon-alpha Receptor Monoclonal Antibody, in Moderate-to-Severe Systemic Lupus Erythematosus’, Arthritis Rheumatol, 69:376-86.
Furie, R., V. P. Werth, J. F. Merola, L. Stevenson, T. L. Reynolds, H. Naik, W. Wang, R. Christmann, A. Gardet, A. Pellerin, S. Hamann, P. Auluck, C. Barbey, P. Gulati, D. Rabah, and N. Franchimont. 2019. ‘Monoclonal antibody targeting BDCA2 ameliorates skin lesions in systemic lupus erythematosus’, The Journal of clinical investigation, 129:1359-71.
Garbers, Christoph, Sylvia Heink, Thomas Korn, and Stefan Rose-John. 2018. ‘Interleukin-6: designing specific therapeutics for a complex cytokine’, Nature Reviews Drug Discovery, 17:395-412.
Gauthier, J., and C. J. Turtle. 2018. ‘Insights into cytokine release syndrome and neurotoxicity after CD19-specific CAR-T cell therapy’, Curr Res Transl Med, 66:50-52.
Gendelman, H. E., S. Ding, N. Gong, J. Liu, S. H. Ramirez, Y. Persidsky, R. L. Mosley, T. Wang, D. J. Volsky, and H. Xiong. 2009. ‘Monocyte chemotactic protein-1 regulates voltage-gated K+ channels and macrophage transmigration’, J Neuroimmune Pharmacol, 4:47-59.
Gerlach, B., S. M. Cordier, A. C. Schmukle, C. H. Emmerich, E. Rieser, T. L. Haas, A. I. Webb, J. A. Rickard, H. Anderton, W. W. Wong, U. Nachbur, L. Gangoda, U. Warnken, A. W. Purcell, J. Silke, and H. Walczak. 2011. ‘Linear ubiquitination prevents inflammation and regulates immune signalling’, Nature, 471:591-6.
Gill, S. E., M. Rohan, and S. Mehta. 2015. ‘Role of pulmonary microvascular endothelial cell apoptosis in murine sepsis-induced lung injury in vivo’, Respir Res, 16:015-0266.
Gill, S. E., R. Taneja, M. Rohan, L. Wang, and S. Mehta. 2014. ‘Pulmonary microvascular albumin leak is associated with endothelial cell death in murine sepsis-induced lung injury in vivo’, PloS one, 9.
Girkin, J. L., L. M. Hatchwell, A. M. Collison, M. R. Starkey, P. M. Hansbro, H. Yagita, P. S. Foster, and J. Mattes. 2017. ‘TRAIL signaling is proinflammatory and proviral in a murine model of rhinovirus 1B infection’, Am J Physiol Lung Cell Mol Physiol, 312: L89-199.
Gomez-Garcia, F., J. L. Sanz-Cabanillas, I. Viguera-Guerra, B. Isla-Tejera, A. V. Nieto, and J. Ruano. 2018. ‘Scoping Review on Use of Drugs Targeting Interleukin 1 Pathway in DIRA and DITRA’, Dermatol Ther (Heidelb), 8:539-56.
Gottlieb, R. A. 2011. ‘Cell death pathways in acute ischemia/reperfusion injury’, J Cardiovasc Pharmacol Ther, 16:233-8.
Greenhill, C. J., S. Rose-John, R. Lissilaa, W. Ferlin, M. Ernst, P. J. Hertzog, A. Mansell, and B. J. Jenkins. 2011. ‘IL-6 trans-signaling modulates TLR4-dependent inflammatory responses via STAT3’, J Immunol, 186:1199-208.
Griffith, T. S., S. R. Wiley, M. Z. Kubin, L. M. Sedger, C. R. Maliszewski, and N. A. Fanger. 1999. ‘Monocyte-mediated tumoricidal activity via the tumor necrosis factor-related cytokine, TRAIL’, The Journal of experimental medicine, 189:1343-54.
Griffiths, Mark J D, Danny Francis McAuley, Gavin D Perkins, Nicholas Barrett, Bronagh Blackwood, Andrew Boyle, Nigel Chee, Bronwen Connolly, Paul Dark, Simon Finney, Aemun Salam, Jonathan Silversides, Nick Tarmey, Matt P Wise, and Simon V Baudouin. 2019. ‘Guidelines on the management of acute respiratory distress syndrome’, BMJ Open Respiratory Research, 6: e000420.
Gringhuis, Sonja I., Tanja M. Kaptein, Brigitte A. Wevers, Bart Theelen, Michiel van der Vlist, Teun Boekhout, and Teunis B. H. Geijtenbeek. 2012. ‘Dectin-1 is an extracellular pathogen sensor for the induction and processing of IL-1ß via a noncanonical caspase-8 inflammasome’, Nature Immunology, 13:246-54.
Guan, W. J., Z. Y. Ni, Y. Hu, W. H. Liang, C. Q. Ou, J. X. He, L. Liu, H. Shan, C. L. Lei, D. S. C. Hui, B. Du, L. J. Li, G. Zeng, K. Y. Yuen, R. C. Chen, C. L. Tang, T. Wang, P. Y. Chen, J. Xiang, S. Y. Li, J. L. Wang, Z. J. Liang, Y. X. Peng, L. Wei, Y. Liu, Y. H. Hu, P. Peng, J. M. Wang, J. Y. Liu, Z. Chen, G. Li, Z. J. Zheng, S. Q. Qiu, J. Luo, C. J. Ye, S. Y. Zhu, and N. S. Zhong. 2020. ‘Clinical Characteristics of Coronavirus Disease 2019 in China’, N Engl J Med, 28.
Guicciardi, Maria Eugenia, Justin L Mott, Steven F Bronk, Satoshi Kurita, Christian D Fingas, and Gregory J Gores. 2011. ‘Cellular inhibitor of apoptosis 1 (clAP-1) degradation by caspase 8 during TNF-related apoptosis-inducing ligand (TRAIL)-induced apoptosis’, Experimental cell research, 317:107-16.
Gurung, Prajwal, Paras K. Anand, R. K. Subbarao Malireddi, Lieselotte Vande Walle, Nina Van Opdenbosch, Christopher P. Dillon, Ricardo Weinlich, Douglas R. Green, Mohamed Lamkanfi, and Thirumala-Devi Kanneganti. 2014. ‘FADD and Caspase-8 Mediate Priming and Activation of the Canonical and Noncanonical Nlrp3 Inflammasomes’, The Journal of Immunology, 192:1835-46.
Hagimoto, Naoki, Kazuyoshi Kuwano, Hiroyuki Miyazaki, Ritsuko Kunitake, Masaki Fujita, Masayuki Kawasaki, Yumi Kaneko, and Nobuyuki Hara. 1997. ‘Induction of apoptosis and pulmonary fibrosis in mice in response to ligation of Fas antigen’, American journal of respiratory cell and molecular biology, 17:272-78.
Hamilton, G. L., H. Chen, G. Deshmukh, C. Eigenbrot, R. Fong, A. Johnson, P. B. Kohli, P. J. Lupardus, B. M. Liederer, S. Ramaswamy, H. Wang, J. Wang, Z. Xu, Y. Zhu, D. Vucic, and S. Patel. 2019. ‘Potent and selective inhibitors of receptor-interacting protein kinase 1 that lack an aromatic back pocket group’, Bioorg Med Chem Lett, 29:1497-501.
Han, S., and R. K. Mallampalli. 2015. ‘The acute respiratory distress syndrome: from mechanism to translation’, J Immunol, 194:855-60.
Harris, P. A., S. B. Berger, J. U. Jeong, R. Nagilla, D. Bandyopadhyay, N. Campobasso, C. A. Capriotti, J. A. Cox, L. Dare, X. Dong, P. M. Eidam, J. N. Finger, S. J. Hoffman, J. Kang, V. Kasparcova, B. W. King, R. Lehr, Y. Lan, L. K. Leister, J. D. Lich, T. T. MacDonald, N. A. Miller, M. T. Ouellette, C. S. Pao, A. Rahman, M. A. Reilly, A. R. Rendina, E. J. Rivera, M. C. Schaeffer, C. A. Sehon, R. R. Singhaus, H. H. Sun, B. A. Swift, R. D. Totoritis, A. Vossenkamper, P. Ward, D. D. Wisnoski, D. Zhang, R. W. Marquis, P. J. Gough, and J. Bertin. 2017. ‘Discovery of a First-in-Class Receptor Interacting Protein 1 (RIP1) Kinase Specific Clinical Candidate (GSK2982772) for the Treatment of Inflammatory Diseases’, J Med Chem, 60:1247-61.
Hartwig, T., A. Montinaro, S. von Karstedt, A. Sevko, S. Surinova, A. Chakravarthy, L. Taraborrelli, P. Draber, E. Lafont, F. Arce Vargas, M. A. El-Bahrawy, S. A. Quezada, and H. Walczak. 2017. ‘The TRAIL-Induced Cancer Secretome Promotes a Tumor-Supportive Immune Microenvironment via CCR2’, Mol Cell, 65:730-42.e5.
Hashimoto, S., A. Kobayashi, K. Kooguchi, Y. Kitamura, H. Onodera, and H. Nakajima. 2000. ‘Upregulation of two death pathways of perforin/granzyme and FasL/Fas in septic acute respiratory distress syndrome’, Am J Respir Crit Care Med, 161:237-43.
Haybaeck, J., N. Zeller, M. J. Wolf, A. Weber, U. Wagner, M. O. Kurrer, J. Bremer, G. Iezzi, R. Graf, P. A. Clavien, R. Thimme, H. Blum, S. A. Nedospasov, K. Zatloukal, M. Ramzan, S. Ciesek, T. Pietschmann, P. N. Marche, M. Karin, M. Kopf, J. L. Browning, A. Aguzzi, and M. Heikenwalder. 2009. ‘A lymphotoxin-driven pathway to hepatocellular carcinoma’, Cancer Cell, 16:295-308.
Hehlgans, T., B. Stoelcker, P. Stopfer, P. Müller, G. Cernaianu, M. Guba, M. Steinbauer, S. A. Nedospasov, K. Pfeffer, and D. N. Männel. 2002. ‘Lymphotoxin-beta receptor immune interaction promotes tumor growth by inducing angiogenesis’, Cancer Res, 62:4034-40.
Herold, S., S. Ludwig, S. Pleschka, and T. Wolff. 2012. ‘Apoptosis signaling in influenza virus propagation, innate host defense, and lung injury’, J Leukoc Biol, 92:75-82.
Herold, S., M. Steinmueller, W. von Wulffen, L. Cakarova, R. Pinto, S. Pleschka, M. Mack, W. A. Kuziel, N. Corazza, T. Brunner, W. Seeger, and J. Lohmeyer. 2008. ‘Lung epithelial apoptosis in influenza virus pneumonia: the role of macrophage-expressed TNF-related apoptosis-inducing ligand’, The Journal of experimental medicine, 205:3065-77.
Hoglen, N. C., L. S. Chen, C. D. Fisher, B. P. Hirakawa, T. Groessl, and P. C. Contreras. 2004. ‘Characterization of IDN-6556 (3-[2-(2-tert-butyl-phenylaminooxalyl)-amino]-propionylamino]-4-oxo-5-(2,3,5,6-tetrafluoro-phenoxy)-pentanoic acid): a liver-targeted caspase inhibitor’, J Pharmacol Exp Ther, 309:634-40.
Hogner, K., T. Wolff, S. Pleschka, S. Plog, A. D. Gruber, U. Kalinke, H. D. Walmrath, J. Bodner, S. Gattenlohner, P. Lewe-Schlosser, M. Matrosovich, W. Seeger, J. Lohmeyer, and S. Herold. 2013. ‘Macrophage-expressed IFN-beta contributes to apoptotic alveolar epithelial cell injury in severe influenza virus pneumonia’, PLOS Pathog, 9:28.
Hopfner, K. P., and V. Hornung. 2020. ‘Molecular mechanisms and cellular functions of cGAS-STING signalling’, Nat Rev Mol Cell Biol, 21:501-21.
Horby, Peter, Wei Shen Lim, Jonathan Emberson, Marion Mafham, Jennifer Bell, Louise Linsell, Natalie Staplin, Christopher Brightling, Andrew Ustianowski, Einas Elmahi, Benjamin Prudon, Christopher Green, Timothy Felton, David Chadwick, Kanchan Rege, Christopher Fegan, Lucy C Chappell, Saul N Faust, Thomas Jaki, Katie Jeffery, Alan Montgomery, Kathryn Rowan, Edmund Juszczak, J Kenneth Baillie, Richard Haynes, and Martin J Landray. 2020. ‘Dexamethasone in Hospitalized Patients with Covid-19—Preliminary Report’, New England Journal of Medicine.
Hou, Jue, Jie Ju, Zili Zhang, Cong Zhao, Zhanhui Li, Jiyue Zheng, Tian Sheng, Hongjian Zhang, Linkun Hu, Xiaoliang Yu, Wei Zhang, Yangxin Li, Meng Wu, Haikuo Ma, Xiaohu Zhang, and Sudan He. 2019. ‘Discovery of potent necroptosis inhibitors targeting RIPK1 kinase activity for the treatment of inflammatory disorder and cancer metastasis’, Cell Death & Disease, 10:493-93.
Hu, J. J., X. Liu, S. Xia, Z. Zhang, Y. Zhang, J. Zhao, J. Ruan, X. Luo, X. Lou, Y. Bai, J. Wang, L. R. Hollingsworth, V. G. Magupalli, L. Zhao, H. R. Luo, J. Kim, J. Lieberman, and H. Wu. 2020. ‘FDA-approved disulfiram inhibits pyroptosis by blocking gasdermin D pore formation’, Nat Immunol, 21:736-45.
Huang, C., Y. Wang, X. Li, L. Ren, J. Zhao, Y. Hu, L. Zhang, G. Fan, J. Xu, X. Gu, Z. Cheng, T. Yu, J. Xia, Y. Wei, W. Wu, X. Xie, W. Yin, H. Li, M. Liu, Y. Xiao, H. Gao, L. Guo, J. Xie, G. Wang, R. Jiang, Z. Gao, Q. Jin, J. Wang, and B. Cao. 2020. ‘Clinical features of patients infected with 2019 novel coronavirus in Wuhan, China’, Lancet, 395:497-506.
Huet, T., H. Beaussier, O. Voisin, S. Jouveshomme, G. Dauriat, I. Lazareth, E. Sacco, J. M. Naccache, Y. Bezie, S. Laplanche, A. Le Berre, J. Le Pavec, S. Salmeron, J. Emmerich, J. J. Mourad, G. Chatellier, and G. Hayem. 2020. ‘Anakinra for severe forms of COVID-19: a cohort study’, Lancet Rheumatol, 2: e393-e400.
Israelow, Benjamin, Eric Song, Tianyang Mao, Peiwen Lu, Amit Meir, Feimei Liu, Mia Madel Alfajaro, Jin Wei, Huiping Dong, Robert J. Homer, Aaron Ring, Craig B. Wilen, and Akiko Iwasaki. 2020. ‘Mouse model of SARS-CoV-2 reveals inflammatory role of type I interferon signaling’, bioRxiv: the preprint server for biology: 2020.05.27.118893.
Ji, P., J. Chen, A. Golding, N. P. Nikolov, B. Saluja, Y. R. Ren, and C. G. Sahajwalla. 2020. ‘Immunomodulatory Therapeutic Proteins in COVID-19: Current Clinical Development and Clinical Pharmacology Considerations’, J Clin Pharmacol, 60:1275-93.
Jia, Z., C. Xu, J. Shen, T. Xia, J. Yang, and Y. He. 2015. ‘The natural compound celastrol inhibits necroptosis and alleviates ulcerative colitis in mice’, Int Immunopharmacol, 29:552-59.
Jiao, H., L. Wachsmuth, S. Kumari, R. Schwarzer, J. Lin, R. O. Eren, A. Fisher, R. Lane, G. R. Young, G. Kassiotis, W. J. Kaiser, and M. Pasparakis. 2020. ‘Z-nucleic-acid sensing triggers ZBP1-dependent necroptosis and inflammation’, Nature, 580:391-95.
Kalil, A. C., and P. G. Thomas. 2019. ‘Influenza virus-related critical illness: pathophysiology and epidemiology’, Crit Care, 23:019-2539.
Kapur, S., and M. E. Bonk. 2009. ‘Rilonacept (arcalyst), an interleukin-1 trap for the treatment of cryopyrin-associated periodic syndromes’, P T, 34:138-41.
Karikó, K., H. Ni, J. Capodici, M. Lamphier, and D. Weissman. 2004. ‘mRNA is an endogenous ligand for Toll-like receptor 3’, The Journal of biological chemistry, 279:12542-50.
Katalan, S., R. Falach, A. Rosner, M. Goldvaser, T. Brosh-Nissimov, A. Dvir, A. Mizrachi, O. Goren, B. Cohen, Y. Gal, A. Sapoznikov, S. Ehrlich, T. Sabo, and C. Kronman. 2017. ‘A novel swine model of ricin-induced acute respiratory distress syndrome’, Dis Model Mech, 10:173-83.
Katsikis, P. D., M. E. García-Ojeda, J. F. Torres-Roca, D. R. Greenwald, and L. A. Herzenberg. 1997. ‘HIV type 1 Tat protein enhances activation—but not Fas (CD95)-induced peripheral blood T cell apoptosis in healthy individuals’, Int Immunol, 9:835-41.
Katsikis, P. D., M. E. García-Ojeda, E. S. Wunderlich, C. A. Smith, H. Yagita, K. Okumura, N. Kayagaki, M. Alderson, and L. A. Herzenberg. 1996. ‘Activation-induced peripheral blood T cell apoptosis is Fas independent in HIV-infected individuals’, Int Immunol, 8:1311-7.
Kennedy, William P., J. Abraham Simon, Carolyn Offutt, Priscilla Horn, Ann Herman, Michael J. Townsend, Meina T. Tang, Jane L. Grogan, Frank Hsieh, and John C. Davis. 2014. ‘Efficacy and safety of pateclizumab (anti-lymphotoxin-α) compared to adalimumab in rheumatoid arthritis: a head-to-head phase 2 randomized controlled study (The ALTARA Study)’, Arthritis Research & Therapy, 16:467.
Kiltz, Uta, David Kiefer, Jürgen Braun, Eduardo Jorge Schiffrin, Charlotte Girard-Guyonvarc'h, and Cem Gabay. 2020. ‘Prolonged treatment with Tadekinig alfa in adult-onset Still's disease’, Annals of the Rheumatic Diseases, 79: e10-e10.
Kitamura, Y., S. Hashimoto, N. Mizuta, A. Kobayashi, K. Kooguchi, I. Fujiwara, and H. Nakajima. 2001. ‘Fas/FasL-dependent apoptosis of alveolar cells after lipopolysaccharide-induced lung injury in mice’, Am J Respir Crit Care Med, 163:762-9.
Knickelbein, J. E., W. R. Tucker, N. Bhatt, K. Armbrust, D. Valent, D. Obiyor, R. B. Nussenblatt, and H. N. Sen. 2016. ‘Gevokizumab in the Treatment of Autoimmune Non-necrotizing Anterior Scleritis: Results of a Phase I/II Clinical Trial’, Am J Ophthalmol, 172:104-10.
Kreer, Christoph, Matthias Zehner, Timm Weber, Meryem S. Ercanoglu, Lutz Gieselmann, Cornelius Rohde, Sandro Halwe, Michael Korenkov, Philipp Schommers, Kanika Vanshylla, Veronica Di Cristanziano, Hanna Janicki, Reinhild Brinker, Artem Ashurov, Verena Krähling, Alexandra Kupke, Hadas Cohen-Dvashi, Manuel Koch, Jan Mathis Eckert, Simone Lederer, Nico Pfeifer, Timo Wolf, Maria J. G. T. Vehreschild, Clemens Wendtner, Ron Diskin, Henning Gruell, Stephan Becker, and Florian Klein. 2020. ‘Longitudinal Isolation of Potent Near-Germline SARS-CoV-2-Neutralizing Antibodies from COVID-19 Patients’, Cell, 182:843-54.e12.
Kumari, S., Y. Redouane, J. Lopez-Mosqueda, R. Shiraishi, M. Romanowska, S. Lutzmayer, J. Kuiper, C. Martinez, I. Dikic, M. Pasparakis, and F. Ikeda. 2014. ‘Sharpin prevents skin inflammation by inhibiting TNFR1-induced keratinocyte apoptosis’, Elife, 3.
Kuwano, K., N. Hagimoto, M. Kawasaki, T. Yatomi, N. Nakamura, S. Nagata, T. Suda, R. Kunitake, T. Maeyama, H. Miyazaki, and N. Hara. 1999. ‘Essential roles of the Fas-Fas ligand pathway in the development of pulmonary fibrosis’, The Journal of clinical investigation, 104:13-9.
Kuwano, K., M. Kawasaki, T. Maeyama, N. Hagimoto, N. Nakamura, K. Shirakawa, and N. Hara. 2000. ‘Soluble form of fas and fas ligand in BAL fluid from patients with pulmonary fibrosis and bronchiolitis obliterans organizing pneumonia’, Chest, 118:451-8.
Leclerc, M., S. Naserian, C. Pilon, A. Thiolat, G. H. Martin, C. Pouchy, C. Dominique, Y. Belkacemi, F. Charlotte, S. Maury, B. L. Salomon, and J. L. Cohen. 2016. ‘Control of GVHD by regulatory T cells depends on TNF produced by T cells and TNFR2 expressed by regulatory T cells’, Blood, 128:1651-9.
Lee, D. W., J. N. Kochenderfer, M. Stetler-Stevenson, Y. K. Cui, C. Delbrook, S. A. Feldman, T. J. Fry, R. Orentas, M. Sabatino, N. N. Shah, S. M. Steinberg, D. Stroncek, N. Tschernia, C. Yuan, H. Zhang, L. Zhang, S. A. Rosenberg, A. S. Wayne, and C. L. Mackall. 2015. ‘T cells expressing CD19 chimeric antigen receptors for acute lymphoblastic leukaemia in children and young adults: a phase 1 dose-escalation trial’, Lancet, 385:517-28.
Lee, K. S., Y. H. Choi, Y. S. Kim, S. H. Baik, Y. J. Oh, S. S. Sheen, J. H. Park, S. C. Hwang, and K. J. Park. 2008. ‘Evaluation of bronchoalveolar lavage fluid from ARDS patients with regard to apoptosis’, Respir Med, 102:464-9.
Lesokhin, A. M., T. M. Hohl, S. Kitano, C. Cortez, D. Hirschhorn-Cymerman, F. Avogadri, G. A. Rizzuto, J. J. Lazarus, E. G. Pamer, A. N. Houghton, T. Merghoub, and J. D. Wolchok. 2012. ‘Monocytic CCR2 (+) myeloid-derived suppressor cells promote immune escape by limiting activated CD8 T-cell infiltration into the tumor microenvironment’, Cancer Res, 72:876-86.
Li, D., C. Li, L. Li, S. Chen, L. Wang, Q. Li, X. Wang, X. Lei, and Z. Shen. 2016. ‘Natural Product Kongensin A is a Non-Canonical HSP90 Inhibitor that Blocks RIP3-dependent Necroptosis’, Cell Chem Biol, 23:257-66.
Li, Juyi, Xiufang Wang, Jian Chen, Xiuran Zuo, Hongmei Zhang, and Aiping Deng. 2020. ‘COVID-19 infection may cause ketosis and ketoacidosis’, Diabetes, Obesity and Metabolism, n/a.
Linkermann, A., J. H. Brasen, N. Himmerkus, S. Liu, T. B. Huber, U. Kunzendorf, and S. Krautwald. 2012. ‘Rip1 (receptor-interacting protein kinase 1) mediates necroptosis and contributes to renal ischemia/reperfusion injury’, Kidney Int, 81:751-61.
Liu, Furong, Xin Long, Wenbin Zou, Minghao Fang, Wenjuan Wu, Wei Li, Bixiang Zhang, Wanguang Zhang, Xiaoping Chen, and Zhanguo Zhang. 2020. ‘Highly ACE2 Expression in Pancreas May Cause Pancreas Damage After SARS-CoV-2 Infection’, medRxiv: 2020.02.28.20029181.
Liu, Y., Y. Fang, X. Chen, Z. Wang, X. Liang, T. Zhang, M. Liu, N. Zhou, J. Lv, K. Tang, J. Xie, Y. Gao, F. Cheng, Y. Zhou, Z. Zhang, Y. Hu, X. Zhang, Q. Gao, Y. Zhang, and B. Huang. 2020. ‘Gasdermin E-mediated target cell pyroptosis by CAR T cells triggers cytokine release syndrome’, Sci Immunol, 5.
Lopez, A. D., S. Avasarala, S. Grewal, A. K. Murali, and L. London. 2009. ‘Differential role of the Fas/Fas ligand apoptotic pathway in inflammation and lung fibrosis associated with reovirus 1/L-induced bronchiolitis obliterans organizing pneumonia and acute respiratory distress syndrome’, J Immunol, 183:8244-57.
Ma, R., T. P. Ortiz Serrano, J. Davis, A. D. Prigge, and K. M. Ridge. 2020. ‘The cGAS-STING pathway: The role of self-DNA sensing in inflammatory lung disease’, FASEB J.
Mangan, Matthew S. J., Edward J. Olhava, William R. Roush, H. Martin Seidel, Gary D. Glick, and Eicke Latz. 2018. ‘Targeting the NLRP3 inflammasome in inflammatory diseases’, Nature Reviews Drug Discovery, 17:588-606.
Martinez-Lostao, L., A. Anel, and J. Pardo. 2015. ‘How Do Cytotoxic Lymphocytes Kill Cancer Cells?’, Clin Cancer Res, 21:5047-56.
Matsushima, K., C. G. Larsen, G. C. DuBois, and J. J. Oppenheim. 1989. ‘Purification and characterization of a novel monocyte chemotactic and activating factor produced by a human myelomonocytic cell line’, The Journal of experimental medicine, 169:1485-90.
Matthay, M. A., J. M. Aldrich, and J. E. Gotts. 2020. ‘Treatment for severe acute respiratory distress syndrome from COVID-19’, Lancet Respir Med, 8:433-34.
Matthay, M. A., R. L. Zemans, G. A. Zimmerman, Y. M. Arabi, J. R. Beitler, A. Mercat, M. Herridge, A. G. Randolph, and C. S. Calfee. 2019. ‘Acute respiratory distress syndrome’, Nat Rev Dis Primers, 5:18.
Matute-Bello, G., W. C. Liles, C. W. Frevert, M. Nakamura, K. Ballman, C. Vathanaprida, P. A. Kiener, and T. R. Martin. 2001. ‘Recombinant human Fas ligand induces alveolar epithelial cell apoptosis and lung injury in rabbits’, Am J Physiol Lung Cell Mol Physiol, 281.
Matute-Bello, G., W. C. Liles, K. P. Steinberg, P. A. Kiener, S. Mongovin, E. Y. Chi, M. Jonas, and T. R. Martin. 1999. ‘Soluble Fas ligand induces epithelial cell apoptosis in humans with acute lung injury (ARDS)’, J Immunol, 163:2217-25.
Matute-Bello, G., R. K. Winn, M. Jonas, E. Y. Chi, T. R. Martin, and W. C. Liles. 2001. ‘Fas (CD95) induces alveolar epithelial cell apoptosis in vivo: implications for acute pulmonary inflammation’, The American Journal of Pathology, 158:153-61.
Matute-Bello, Gustavo, Robert K Winn, Thomas R Martin, and W Conrad Liles. 2004. ‘Sustained lipopolysaccharide-induced lung inflammation in mice is attenuated by functional deficiency of the Fas/Fas ligand system’, Clinical and diagnostic laboratory immunology, 11:358-61.
Maude, S. L., D. Barrett, D. T. Teachey, and S. A. Grupp. 2014. ‘Managing cytokine release syndrome associated with novel T cell-engaging therapies’, Cancer J, 20:119-22.
Maude, S. L., N. Frey, P. A. Shaw, R. Aplenc, D. M. Barrett, N. J. Bunin, A. Chew, V. E. Gonzalez, Z. Zheng, S. F. Lacey, Y. D. Mahnke, J. J. Melenhorst, S. R. Rheingold, A. Shen, D. T. Teachey, B. L. Levine, C. H. June, D. L. Porter, and S. A. Grupp. 2014. ‘Chimeric antigen receptor T cells for sustained remissions in leukemia’, N Engl J Med, 371:1507-17.
McDermott, Jason E., Hugh D. Mitchell, Lisa E. Gralinski, Amie J. Eisfeld, Laurence Josset, Armand Bankhead, 3rd, Gabriele Neumann, Susan C. Tilton, Alexandra Schäfer, Chengjun Li, Shufang Fan, Shannon McWeeney, Ralph S. Baric, Michael G. Katze, and Katrina M. Waters. 2016. ‘The effect of inhibition of PP1 and TNFα signaling on pathogenesis of SARS coronavirus’, BMC systems biology, 10:93-93.
Memish, Ziad A., Stanley Perlman, Maria D. Van Kerkhove, and Alimuddin Zumla. 2020. ‘Middle East respiratory syndrome’, The Lancet, 395:1063-77.
Moore, R. J., D. M. Owens, G. Stamp, C. Arnott, F. Burke, N. East, H. Holdsworth, L. Turner, B. Rollins, M. Pasparakis, G. Kollias, and F. Balkwill. 1999. ‘Mice deficient in tumor necrosis factor-alpha are resistant to skin carcinogenesis’, Nat Med, 5:828-31.
Müller, M., J. Briscoe, C. Laxton, D. Guschin, A. Ziemiecki, O. Silvennoinen, A. G. Harpur, G. Barbieri, B. A. Witthuhn, C. Schindler, and et al. 1993. ‘The protein tyrosine kinase JAK1 complements defects in interferon-alpha/beta and -gamma signal transduction’, Nature, 366:129-35.
Munroe, M. E., and G. A. Bishop. 2004. ‘Role of tumor necrosis factor (TNF) receptor-associated factor 2 (TRAF2) in distinct and overlapping CD40 and TNF receptor 2/CD120b-mediated B lymphocyte activation’, The Journal of biological chemistry, 279:53222-31.
Murthy, Hemant, Madiha Iqbal, Julio C. Chavez, and Mohamed A. Kharfan-Dabaja. 2019. ‘Cytokine Release Syndrome: Current Perspectives’, Immuno Targets and therapy, 8:43-52.
Nakamura, K., S. Kageyama, and J. W. Kupiec-Weglinski. 2019. ‘Innate immunity in ischemia-reperfusion injury and graft rejection’, Curr Opin Organ Transplant, 24:687-93.
NIH. 2020. “COVID-19 Treatment Guidelines.” In, edited by National Institutes of Health.
Nisihara, T., Y. Ushio, H. Higuchi, N. Kayagaki, N. Yamaguchi, K. Soejima, S. Matsuo, H. Maeda, Y. Eda, K. Okumura, and H. Yagita. 2001. ‘Humanization and epitope mapping of neutralizing anti-human Fas ligand monoclonal antibodies: structural insights into Fas/Fas ligand interaction’, J Immunol, 167:3266-75.
Noda, T., R. Iwakiri, K. Fujimoto, S. Matsuo, and T. Y. Aw. 1998. ‘Programmed cell death induced by ischemia-reperfusion in rat intestinal mucosa’, Am J Physiol, 274.
Nye, Steven, Richard J Whitley, and Michele Kong. 2016. ‘Viral infection in the development and progression of pediatric acute respiratory distress syndrome’, Frontiers in pediatrics, 4:128.
Or, Yvonne Y-Y, Grace T-Y Chung, Ka-Fai To, Chit Chow, Kwong-Wai Choy, Carol Y-K Tong, Alice W-C Leung, Angela B-Y Hui, Sai-Wah Tsao, and Ho-Keung Ng. 2010. ‘Identification of a novel 12p13. 3 amplicon in nasopharyngeal carcinoma’, The Journal of Pathology: A Journal of the Pathological Society of Great Britain and Ireland, 220:97-107.
Orning, P., E. Lien, and K. A. Fitzgerald. 2019. ‘Gasdermins and their role in immunity and inflammation’, The Journal of experimental medicine, 216:2453-65.
Orning, Pontus, Dan Weng, Kristian Starheim, Dmitry Ratner, Zachary Best, Bettina Lee, Alexandria Brooks, Shiyu Xia, Hao Wu, Michelle A. Kelliher, Scott B. Berger, Peter J. Gough, John Bertin, Megan M. Proulx, Jon D. Goguen, Nobuhiko Kayagaki, Katherine A. Fitzgerald, and Egil Lien. 2018. ‘Pathogen blockade of TAK1 triggers caspase-8-dependent cleavage of gasdermin D and cell death’, Science, 362:1064-69.
Owyang, A. M., H. Issafras, J. Corbin, K. Ahluwalia, P. Larsen, E. Pongo, M. Handa, A. H. Horwitz, M. K. Roell, M. Haak-Frendscho, and L. Masat. 2011. ‘XOMA 052, a potent, high-affinity monoclonal antibody for the treatment of IL-1beta-mediated diseases’, MAbs, 3:49-60.
Park, Jae H., Isabelle Rivière, Mithat Gonen, Xiuyan Wang, Brigitte Sénéchal, Kevin J. Curran, Craig Sauter, Yongzeng Wang, Bianca Santomasso, Elena Mead, Mikhail Roshal, Peter Maslak, Marco Davila, Renier J. Brentjens, and Michel Sadelain. 2018. ‘Long-Term Follow-up of CD19 CAR Therapy in Acute Lymphoblastic Leukemia’, The New England journal of medicine, 378:449-59.
Peltzer, N., M. Darding, A. Montinaro, P. Draber, H. Draberova, S. Kupka, E. Rieser, A. Fisher, C. Hutchinson, L. Taraborrelli, T. Hartwig, E. Lafont, T. L. Haas, Y. Shimizu, C. Boiers, A. Sarr, J. Rickard, S. Alvarez-Diaz, M. T. Ashworth, A. Beal, T. Enver, J. Bertin, W. Kaiser, A. Strasser, J. Silke, P. Bouillet, and H. Walczak. 2018. ‘LUBAC is essential for embryogenesis by preventing cell death and enabling haematopoiesis’, Nature, 557:112-17.
Perl, M., C. S. Chung, U. Perl, J. Lomas-Neira, M. de Paepe, W. G. Cioffi, and A. Ayala. 2007. ‘Fas-induced pulmonary apoptosis and inflammation during indirect acute lung injury’, Am J Respir Crit Care Med, 176:591-601.
Peteranderl, C., L. Morales-Nebreda, B. Selvakumar, E. Lecuona, I. Vadasz, R. E. Morty, C. Schmoldt, J. Bespalowa, T. Wolff, S. Pleschka, K. Mayer, S. Gattenloehner, L. Fink, J. Lohmeyer, W. Seeger, J. I. Sznajder, G. M. Mutlu, G. R. Budinger, and S. Herold. 2016. ‘Macrophage-epithelial paracrine crosstalk inhibits lung edema clearance during influenza infection’, The Journal of clinical investigation, 126:1566-80.
Pikarsky, E., R. M. Porat, I. Stein, R. Abramovitch, S. Amit, S. Kasem, E. Gutkovich-Pyest, S. Urieli-Shoval, E. Galun, and Y. Ben-Neriah. 2004. ‘NF-kappaB functions as a tumour promoter in inflammation-associated cancer’, Nature, 431:461-6.
Platten, M., P. P. Ho, S. Youssef, P. Fontoura, H. Garren, E. M. Hur, R. Gupta, L. Y. Lee, B. A. Kidd, W. H. Robinson, R. A. Sobel, M. L. Selley, and L. Steinman. 2005. ‘Treatment of autoimmune neuroinflammation with a synthetic tryptophan metabolite’, Science, 310:850-5.
Poonia, Bhawna, Maria S. Salvato, Hideo Yagita, Toshihiro Maeda, Ko Okumura, and C. David Pauza. 2009. ‘Treatment with anti-FasL antibody preserves memory lymphocytes and virus-specific cellular immunity in macaques challenged with simian immunodeficiency virus’, Blood, 114:1196-204.
Popivanova, B. K., K. Kitamura, Y. Wu, T. Kondo, T. Kagaya, S. Kaneko, M. Oshima, C. Fujii, and N. Mukaida. 2008. ‘Blocking TNF-alpha in mice reduces colorectal carcinogenesis associated with chronic colitis’, The Journal of clinical investigation, 118:560-70.
Ratziu, V., M. Y. Sheikh, A. J. Sanyal, J. K. Lim, H. Conjeevaram, N. Chalasani, M. Abdelmalek, A. Bakken, C. Renou, M. Palmer, R. A. Levine, B. R. Bhandari, M. Cornpropst, W. Liang, B. King, E. Mondou, F. S. Rousseau, J. McHutchison, and M. Chojkier. 2012. ‘A phase 2, randomized, double-blind, placebo-controlled study of GS-9450 in subjects with nonalcoholic steatohepatitis’, Hepatology, 55:419-28.
Rauert, Hilka, Andreas Wicovsky, Nicole Müller, Daniela Siegmund, Volker Spindler, Jens Waschke, Christian Kneitz, and Harald Wajant. 2010. ‘Membrane tumor necrosis factor (TNF) induces p100 processing via TNF receptor-2 (TNFR2)’, The Journal of biological chemistry, 285:7394-404.
Rickard, J. A., H. Anderton, N. Etemadi, U. Nachbur, M. Darding, N. Peltzer, N. Lalaoui, K. E. Lawlor, H. Vanyai, C. Hall, A. Bankovacki, L. Gangoda, W. W. Wong, J. Corbin, C. Huang, E. S. Mocarski, J. M. Murphy, W. S. Alexander, A. K. Voss, D. L. Vaux, W. J. Kaiser, H. Walczak, and J. Silke. 2014. ‘TNFR1-dependent cell death drives inflammation in Sharpin-deficient mice’, Elife, 3.
Ridker, P. M. 2019. ‘Anticytokine Agents: Targeting Interleukin Signaling Pathways for the Treatment of Atherothrombosis’, Circ Res, 124:437-50.
Roberts, A., D. Deming, C. D. Paddock, A. Cheng, B. Yount, L. Vogel, B. D. Herman, T. Sheahan, M. Heise, G. L. Genrich, S. R. Zaki, R. Baric, and K. Subbarao. 2007. ‘A mouse-adapted SARS-coronavirus causes disease and mortality in BALB/c mice’, PLOS Pathog, 3: e5.
Rodrigue-Gervais, I. G., K. Labbe, M. Dagenais, J. Dupaul-Chicoine, C. Champagne, A. Morizot, A. Skeldon, E. L. Brincks, S. M. Vidal, T. S. Griffith, and M. Saleh. 2014. ‘Cellular inhibitor of apoptosis protein clAP2 protects against pulmonary tissue necrosis during influenza virus infection to promote host survival’, Cell Host Microbe, 15:23-35.
Rong, Yinghui, Jennifer Westfall, Dylan Ehrbar, Timothy LaRocca, and Nicholas J. Mantis. 2018. ‘TRAIL (CD253) Sensitizes Human Airway Epithelial Cells to Toxin-Induced Cell Death’, mSphere, 3: e00399-18.
Roos, C., A. Wicovsky, N. Müller, S. Salzmann, T. Rosenthal, H. Kalthoff, A. Trauzold, A. Seher, F. Henkler, C. Kneitz, and H. Wajant. 2010. ‘Soluble and transmembrane TNF-like weak inducer of apoptosis differentially activate the classical and noncanonical NF-kappa B pathway’, J Immunol, 185:1593-605.
Rose-John, S. 2018. ‘Interleukin-6 Family Cytokines’, Cold Spring Harb Perspect Biol, 10.
Rubino, Francesco, Stephanie A. Amiel, Paul Zimmet, George Alberti, Stefan Bornstein, Robert H. Eckel, Geltrude Mingrone, Bernhard Boehm, Mark E. Cooper, Zhonglin Chai, Stefano Del Prato, Linong Ji, David Hopkins, William H. Herman, Kamlesh Khunti, Jean-Claude Mbanya, and Eric Renard. 2020. ‘New-Onset Diabetes in Covid-19’, The New England Journal of Medicine, 383:789-90.
Rudolphi, K., N. Gerwin, N. Verzijl, P. van der Kraan, and W. van den Berg. 2003. ‘Pralnacasan, an inhibitor of interleukin-1beta converting enzyme, reduces joint damage in two murine models of osteoarthritis’, Osteoarthritis Cartilage, 11:738-46.
Ruiz-Ruiz, C. 2000. ‘Interferon-gamma treatment elevates caspase-8 expression and sensitizes human breast tumor cells to a death receptor-induced mitochondria-operated apoptotic program’, Cancer Research.
Saitoh, T., M. Nakayama, H. Nakano, H. Yagita, N. Yamamoto, and S. Yamaoka. 2003. ‘TWEAK induces NF-kappaB2 p100 processing and long lasting NF-kappaB activation’, The Journal of biological chemistry, 278:36005-12.
Salomon, B. L., M. Leclerc, J. Tosello, E. Ronin, E. Piaggio, and J. L. Cohen. 2018. ‘Tumor Necrosis Factor α and Regulatory T Cells in Oncoimmunology’, Front Immunol, 9.
Sarhan, Joseph, Beiyun C. Liu, Hayley I. Muendlein, Peng Li, Rachael Nilson, Amy Y. Tang, Anthony Rongvaux, Stephen C. Bunnell, Feng Shao, Douglas R. Green, and Alexander Poltorak. 2018. ‘Caspase-8 induces cleavage of gasdermin D to elicit pyroptosis during <em>Yersinia</em>infection’, Proceedings of the National Academy of Sciences, 115: E10888-E97.
Sauler, M., I. S. Bazan, and P. J. Lee. 2019. ‘Cell Death in the Lung: The Apoptosis-Necroptosis Axis’, Annu Rev Physiol, 81:375-402.
Schindler, C., K. Shuai, V. R. Prezioso, and J. E. Darnell, Jr. 1992. ‘Interferon-dependent tyrosine phosphorylation of a latent cytoplasmic transcription factor’, Science, 257:809-13.
Schuett, H., R. Oestreich, G. H. Waetzig, W. Annema, M. Luchtefeld, A. Hillmer, U. Bavendiek, J. von Felden, D. Divchev, T. Kempf, K. C. Wollert, D. Seegert, S. Rose-John, U. J. Tietge, B. Schieffer, and K. Grote. 2012. ‘Transsignaling of interleukin-6 crucially contributes to atherosclerosis in mice’, Arterioscler Thromb Vasc Biol, 32:281-90.
Screaton, G. R., J. Mongkolsapaya, X. N. Xu, A. E. Cowper, A. J. McMichael, and J. I. Bell. 1997. ‘TRICK2, a new alternatively spliced receptor that transduces the cytotoxic signal from TRAIL’, Current biology: CB, 7:693-96.
Sedger, L. M., D. M. Shows, R. A. Blanton, J. J. Peschon, R. G. Goodwin, D. Cosman, and S. R. Wiley. 1999. ‘IFN-gamma mediates a novel antiviral activity through dynamic modulation of TRAIL and TRAIL receptor expression’, Journal of immunology (Baltimore, Md.: 1950), 163:920-26.
Shimabukuro-Vornhagen, Alexander, Philipp Godel, Marion Subklewe, Hans Joachim Stemmler, Hans Anton Schlößer, Max Schlaak, Matthias Kochanek, Boris Böll, and Michael S. von Bergwelt-Baildon. 2018. ‘Cytokine release syndrome’, Journal for immunotherapy of cancer, 6:56-56.
Shin, E. C., J. M. Ahn, C. H. Kim, Y. Choi, Y. S. Ahn, H. Kim, S. J. Kim, and J. H. Park. 2001. ‘IFN-gamma induces cell death in human hepatoma cells through a TRAIL/death receptor-mediated apoptotic pathway’, Int J Cancer, 93:262-8.
St Clair, E. W., A. N. Baer, C. Wei, G. Noaiseh, A. Parke, A. Coca, T. O. Utset, M. C. Genovese, D. J. Wallace, J. McNamara, K. Boyle, L. Keyes-Elstein, J. L. Browning, N. Franchimont, K. Smith, J. M. Guthridge, I. Sanz, and J. A. James. 2018. ‘Clinical Efficacy and Safety of Baminercept, a Lymphotoxin β Receptor Fusion Protein, in Primary Sjögren's Syndrome: Results From a Phase II Randomized, Double-Blind, Placebo-Controlled Trial’, Arthritis & rheumatology (Hoboken, N.J.), 70:1470-80.
Suganuma, M., S. Okabe, M. W. Marino, A. Sakai, E. Sueoka, and H. Fujiki. 1999. ‘Essential role of tumor necrosis factor alpha (TNF-alpha) in tumor promotion as revealed by TNF-alpha-deficient mice’, Cancer Res, 59:4516-8.
Swanson, K. V., M. Deng, and J. P. Ting. 2019. ‘The NLRP3 inflammasome: molecular activation and regulation to therapeutics’, Nat Rev Immunol, 19:477-89.
Tamm, I., Y. Wang, E. Sausville, D. A. Scudiero, N. Vigna, T. Oltersdorf, and J. C. Reed. 1998. ‘IAP-family protein survivin inhibits caspase activity and apoptosis induced by Fas (CD95), Bax, caspases, and anticancer drugs’, Cancer Res, 58:5315-20.
Taraborrelli, Lucia, Nieves Peltzer, Antonella Montinaro, Sebastian Kupka, Eva Rieser, Torsten Hartwig, Aida Sarr, Maurice Darding, Peter Draber, Tobias L. Haas, Ayse Akarca, Teresa Marafioti, Manolis Pasparakis, John Bertin, Peter J. Gough, Philippe Bouillet, Andreas Strasser, Martin Leverkus, John Silke, and Henning Walczak. 2018. ‘LUBAC prevents lethal dermatitis by inhibiting cell death induced by TNF, TRAIL and CD95L’, Nat Commun, 9:3910.
Teachey, D. T., S. F. Lacey, P. A. Shaw, J. J. Melenhorst, S. L. Maude, N. Frey, E. Pequignot, V. E. Gonzalez, F. Chen, J. Finklestein, D. M. Barrett, S. L. Weiss, J. C. Fitzgerald, R. A. Berg, R. Aplenc, C. Callahan, S. R. Rheingold, Z. Zheng, S. Rose-John, J. C. White, F. Nazimuddin, G. Wertheim, B. L. Levine, C. H. June, D. L. Porter, and S. A. Grupp. 2016. ‘Identification of Predictive Biomarkers for Cytokine Release Syndrome after Chimeric Antigen Receptor T-cell Therapy for Acute Lymphoblastic Leukemia’, Cancer Discov, 6:664-79.
Thompson, B. T., R. C. Chambers, and K. D. Liu. 2017. ‘Acute Respiratory Distress Syndrome’, N Engl J Med, 377:1904-05.
Thorlund, Kristian, Louis Dron, Jay Park, Grace Hsu, Jamie I. Forrest, and Edward J. Mills. 2020. ‘A real-time dashboard of clinical trials for COVID-19’, The Lancet Digital Health, 2: e286-e87.
Toldo, S., and A. Abbate. 2018. ‘The NLRP3 inflammasome in acute myocardial infarction’, Nat Rev Cardiol, 15:203-14.
Tuettenberg, J., M. Seiz, K. M. Debatin, W. Hollburg, M. von Staden, M. Thiemann, B. Hareng, H. Fricke, and C. Kunz. 2012. ‘Pharmacokinetics, pharmacodynamics, safety and tolerability of APG101, a CD95-Fc fusion protein, in healthy volunteers and two glioma patients’, Int Immunopharmacol, 13:93-100.
Tuthill, M. H., A. Montinaro, J. Zinngrebe, K. Prieske, P. Draber, S. Prieske, T. Newsom-Davis, S. von Karstedt, J. Graves, and H. Walczak. 2015. ‘TRAIL-R2-specific antibodies and recombinant TRAIL can synergise to kill cancer cells’, Oncogene, 34:2138-44.
Varfolomeev, E., T. Goncharov, A. V. Fedorova, J. N. Dynek, K. Zobel, K. Deshayes, W. J. Fairbrother, and D. Vucic. 2008. ‘c-IAP1 and c-IAP2 are critical mediators of tumor necrosis factor alpha (TNFalpha)-induced NF-kappaB activation’, J Biol Chem, 283:24295-9.
Walczak, H., M. A. Degli-Esposti, R. S. Johnson, P. J. Smolak, J. Y. Waugh, N. Boiani, M. S. Timour, M. J. Gerhart, K. A. Schooley, C. A. Smith, R. G. Goodwin, and C. T. Rauch. 1997. ‘TRAIL-R2: a novel apoptosis-mediating receptor for TRAIL’, Embo J, 16:5386-97.
Walczak, Henning, Lucia Taraborrelli, and Nieves Peltzer. 2018. “Combination therapeutics.” In.
Wang, J. N., M. M. Liu, F. Wang, B. Wei, Q. Yang, Y. T. Cai, X. Chen, X. Q. Liu, L. Jiang, C. Li, X. W. Hu, J. T. Yu, T. T. Ma, J. Jin, Y. G. Wu, J. Li, and X. M. Meng. 2019. ‘RIPK1 inhibitor Cpd-71 attenuates renal dysfunction in cisplatin-treated mice via attenuating necroptosis, inflammation and oxidative stress’, Clin Sci, 133:1609-27.
Wang, Y., H. Zhang, Z. Wang, Y. Wei, M. Wang, M. Liu, X. Wang, Y. Jiang, G. Shi, D. Zhao, Z. Yang, Z. Ren, J. Li, Z. Zhang, B. Zhang, B. Zong, X. Lou, C. Liu, N. Tao, X. Li, X. Zhang, Y. Guo, Y. Ye, Y. Qi, H. Li, R. Guo, G. Cheng, S. Li, J. Zhang, G. Liu, L. Chai, Q. Lou, X. Cui, E. Gao, Z. Dong, Y. Hu, Y. H. Chen, and Y. Ma. 2020. ‘Blocking the death checkpoint protein TRAIL improves cardiac function after myocardial infarction in monkeys, pigs, and rats’, Sci Transl Med, 12.
Wang, Yeming, Dingyu Zhang, Guanhua Du, Ronghui Du, Jianping Zhao, Yang Jin, Shouzhi Fu, Ling Gao, Zhenshun Cheng, Qiaofa Lu, Yi Hu, Guangwei Luo, Ke Wang, Yang Lu, Huadong Li, Shuzhen Wang, Shunan Ruan, Chengqing Yang, Chunlin Mei, Yi Wang, Dan Ding, Feng Wu, Xin Tang, Xianzhi Ye, Yingchun Ye, Bing Liu, Jie Yang, Wen Yin, Aili Wang, Guohui Fan, Fei Zhou, Zhibo Liu, Xiaoying Gu, Jiuyang Xu, Lianhan Shang, Yi Zhang, Lianjun Cao, Tingting Guo, Yan Wan, Hong Qin, Yushen Jiang, Thomas Jaki, Frederick G. Hayden, Peter W. Horby, Bin Cao, and Chen Wang. 2020. ‘Remdesivir in adults with severe COVID-19: a randomised, double-blind, placebo-controlled, multicentre trial’, The Lancet, 395:1569-78.
Wang, Yin, Weiwei Jiang, Qi He, Cheng Wang, Baoju Wang, Pan Zhou, Nianguo Dong, and Qiaoxia Tong. 2020. ‘Early, low-dose and short-term application of corticosteroid treatment in patients with severe COVID-19 pneumonia: single-center experience from Wuhan, China’, medRxiv: 2020.03.06.20032342.
Weisel, K., N. E. Scott, D. J. Tompson, B. J. Votta, S. Madhavan, K. Povey, A. Wolstenholme, M. Simeoni, T. Rudo, L. Richards-Peterson, T. Sahota, J. G. Wang, J. Lich, J. Finger, A. Verticelli, M. Reilly, P. J. Gough, P. A. Harris, J. Bertin, and M. L. Wang. 2017. ‘Randomized clinical study of safety, pharmacokinetics, and pharmacodynamics of RIPK1 inhibitor GSK2982772 in healthy volunteers’, Pharmacol Res Perspect, 5:365.
Weiskopf, Daniela, Katharina S. Schmitz, Matthijs P. Raadsen, Alba Grifoni, Nisreen M. A. Okba, Henrik Endeman, Johannes P. C. van den Akker, Richard Molenkamp, Marion P. G. Koopmans, Eric C. M. van Gorp, Bart L. Haagmans, Rik L. de Swart, Alessandro Sette, and Rory D. de Vries. 2020. ‘Phenotype and kinetics of SARS-CoV-2-specific T cells in COVID-19 patients with acute respiratory distress syndrome’, Science Immunology, 5: eabd2071.
Weiss, R., M. Sachet, J. Zinngrebe, T. Aschacher, M. Krainer, B. Hegedus, H. Walczak, and M. Bergmann. 2013. ‘IL-24 sensitizes tumor cells to TLR3-mediated apoptosis’, Cell Death & Differentiation, 20:823-33.
Wick, W., H. Fricke, K. Junge, G. Kobyakov, T. Martens, O. Heese, B. Wiestler, M. G. Schliesser, A. von Deimling, J. Pichler, E. Vetlova, I. Harting, J. Debus, C. Hartmann, C. Kunz, M. Platten, M. Bendszus, and S. E. Combs. 2014. ‘A phase II, randomized, study of weekly APG101+reirradiation versus reirradiation in progressive glioblastoma’, Clin Cancer Res, 20:6304-13.
Wick, W., A. Krendyukov, K. Junge, T. Höger, and H. Fricke. 2019. ‘Longitudinal analysis of quality of life following treatment with Asunercept plus reirradiation versus reirradiation in progressive glioblastoma patients’, J Neurooncol, 145:531-40.
Wroblewski, V. J., C. McCloud, K. Davis, J. Manetta, R. Micanovic, and D. R. Witcher. 2003. ‘Pharmacokinetics, metabolic stability, and subcutaneous bioavailability of a genetically engineered analog of DcR3, FLINT [DcR3 (R218Q)], in cynomolgus monkeys and mice’, Drug Metab Dispos, 31:502-7.
Wu, C., X. Chen, Y. Cai, J. Xia, X. Zhou, S. Xu, H. Huang, L. Zhang, C. Du, Y. Zhang, J. Song, S. Wang, Y. Chao, Z. Yang, J. Xu, D. Chen, W. Xiong, L. Xu, F. Zhou, J. Jiang, C. Bai, J. Zheng, and Y. Song. 2020. ‘Risk Factors Associated With Acute Respiratory Distress Syndrome and Death in Patients With Coronavirus Disease 2019 Pneumonia in Wuhan, China’, JAMA Intern Med, 13.
Wurzer, W. J., C. Ehrhardt, S. Pleschka, F. Berberich-Siebelt, T. Wolff, H. Walczak, O. Planz, and S. Ludwig. 2004. ‘NF-kappaB-dependent induction of tumor necrosis factor-related apoptosis-inducing ligand (TRAIL) and Fas/FasL is crucial for efficient influenza virus propagation’, The Journal of biological chemistry, 279:30931-7.
Xu, Z., L. Shi, Y. Wang, J. Zhang, L. Huang, C. Zhang, S. Liu, P. Zhao, H. Liu, L. Zhu, Y. Tai, C. Bai, T. Gao, J. Song, P. Xia, J. Dong, J. Zhao, and F. S. Wang. 2020. ‘Pathological findings of COVID-19 associated with acute respiratory distress syndrome’, Lancet Respir Med.
Yang, Hong, Lisa Kozicky, Aabida Saferali, Shan-Yu Fung, Nicole Afacan, Bing Cai, Reza Falsafi, Erin Gill, Mingyao Liu, and Tobias R Kollmann. 2016. ‘Endosomal pH modulation by peptide-gold nanoparticle hybrids enables potent anti-inflammatory activity in phagocytic immune cells’, Biomaterials, 111:90-102.
Yang, J. K., S. S. Lin, X. J. Ji, and L. M. Guo. 2010. ‘Binding of SARS coronavirus to its receptor damages islets and causes acute diabetes’, Acta Diabetol, 47:193-9.
Yang, X., Y. Yu, J. Xu, H. Shu, J. Xia, H. Liu, Y. Wu, L. Zhang, Z. Yu, M. Fang, T. Yu, Y. Wang, S. Pan, X. Zou, S. Yuan, and Y. Shang. 2020. ‘Clinical course and outcomes of critically ill patients with SARS-CoV-2 pneumonia in Wuhan, China: a single-centered, retrospective, observational study’, Lancet Respir Med, 8:475-81.
Yu, Junhui, Shan Li, Jie Qi, Zilu Chen, Yunhua Wu, Jing Guo, Kai Wang, Xuejun Sun, and Jianbao Zheng. 2019. ‘Cleavage of GSDME by caspase-3 determines lobaplatin-induced pyroptosis in colon cancer cells’, Cell Death & Disease, 10:193.
Zarnegar, B. J., Y. Wang, D. J. Mahoney, P. W. Dempsey, H. H. Cheung, J. He, T. Shiba, X. Yang, W. C. Yeh, T. W. Mak, R. G. Korneluk, and G. Cheng. 2008. ‘Noncanonical NF-kappaB activation requires coordinated assembly of a regulatory complex of the adaptors clAP1, clAP2, TRAF2 and TRAF3 and the kinase NIK’, Nat Immunol, 9:1371-8.
Zemans, R. L., and M. A. Matthay. 2017. ‘What drives neutrophils to the alveoli in ARDS?’, Thorax, 72:1-3.
Zhang, H., P. Neuhöfer, L. Song, B. Rabe, M. Lesina, M. U. Kurkowski, M. Treiber, T. Wartmann, S. Regnér, H. Thorlacius, D. Saur, G. Weirich, A. Yoshimura, W. Halangk, J. P. Mizgerd, R. M. Schmid, S. Rose-John, and H. Algül. 2013. ‘IL-6 trans-signaling promotes pancreatitis-associated lung injury and lethality’, The Journal of clinical investigation, 123:1019-31.
Zheng, Z. H., X. Zeng, X. Y. Nie, Y. J. Cheng, J. Liu, X. X. Lin, H. Yao, C. C. Ji, X. M. Chen, F. Jun, and S. H. Wu. 2019. ‘Interleukin-1 blockade treatment decreasing cardiovascular risk’, Clin Cardiol, 42:942-51.
Zhou, F., T. Yu, R. Du, G. Fan, Y. Liu, Z. Liu, J. Xiang, Y. Wang, B. Song, X. Gu, L. Guan, Y. Wei, H. Li, X. Wu, J. Xu, S. Tu, Y. Zhang, H. Chen, and B. Cao. 2020. ‘Clinical course and risk factors for mortality of adult inpatients with COVID-19 in Wuhan, China: a retrospective cohort study’, Lancet, 395:1054-62.
Zimmermann, J., S. Herrlinger, A. Pruy, T. Metzger, and C. Wanner. 1999. ‘Inflammation enhances cardiovascular risk and mortality in hemodialysis patients’, Kidney Int, 55:648-58.
Zinngrebe, Julia, Eva Rieser, Lucia Taraborrelli, Nieves Peltzer, Torsten Hartwig, Hongwei Ren, Ildikó Kovacs, Cornelia Endres, Peter Draber, Maurice Darding, Silvia von Karstedt, Johannes Lemke, Balazs Dome, Michael Bergmann, Brian J. Ferguson, and Henning Walczak. 2016. ‘LUBAC deficiency perturbs TLR3 signaling to cause immunodeficiency and autoinflammation’, The Journal of experimental medicine, 213:2671-89.

1. A method for treating a subject for a disease selected from acute organ failure, acute respiratory distress syndrome (ARDS), which is optionally induced by an airway targeting virus, or a patho-physiologically related disease, or for treating an organ prior to or after transplantation,
the method comprising administering to the subject or organ a combination treatment of at least 2 agents, the combination comprising:
(a) one or more agents selected from the list consisting of:
a death-ligand inhibiting agent; and/or
an agent which is a RIPK1 kinase inhibitor or RIPK3 inhibitor; and/or
an agent which is a caspase inhibitor; and/or
an agent that inhibits inflammasome activation and/or gasdermin activation; and
(b) one or more anti-inflammatory agents, different to the agent in (a).

2. The method as claimed in claim 1, wherein the ligand in (a) and/or its cognate receptor is selected from the TNF Receptor superfamily members shown in Table 1.

3. The method as claimed in claim 1, wherein the agent (a) is a TRAIL-inhibiting agent.

4. The method as claimed in claim 1, wherein the agent (a) is a FasL-inhibiting agent.

5. The method as claimed in claim 1, wherein the agents in (a) comprise a TRAIL-inhibiting agent and a FasL-inhibiting agent.

6. The method as claimed in claim 1, wherein the ligand in (a) and its cognate receptor are selected from dsRNA/TLR3, LPS/TLR4, TL1A/DR3, cGas/STING.

7. (canceled)

8. The method as claimed in claim 1, comprising an agent (a) which inhibits canonical NLRP3 inflammasome activation or non-canonical inflammasome activation, which is optionally MAS825, OLT1177 or Tranilast.

9. The method as claimed in claim 1, wherein the anti-inflammatory agent is an agent which inhibits the activity of one or more ligands from the TNF Receptor superfamily member shown in Table 1.

10. (canceled)

11. The method as claimed in claim 1, wherein the anti-inflammatory agent is an agent which inhibits IL-6; and/or IL-6R; and/or IL6/sIL6R heterodimer, wherein optionally the agent is the trans-signalling inhibitor sgp130-Fc or an anti-IL-6 antibody or anti-IL-6R antibody which is optionally Tocilizumab.

12. The method as claimed in claim 1, wherein the anti-inflammatory agent is an agent which inhibits the activity of a Caspase, most preferably Caspase-8; RIPK1 kinase or RIPK3 kinase; MLKL; IL-1alpha; and/or IL-1beta.

13. (canceled)

14. (canceled)

15. The method as claimed in claim 1, wherein the anti-inflammatory agent is an agent which inhibits a target in Table 2, and is optionally selected from the inhibitors in Table 2.

16. The method as claimed in claim 1, or claim 15, wherein the anti-inflammatory agent is a TNF- or TNF/LT-alpha-inhibiting agent.

17. The method as claimed in claim 1, or claim 15, wherein the anti-inflammatory agent is an agent which inhibits IL-1alpha- and/or IL-1beta.

18.-20. (canceled)

21. The method as claimed in claim 1, or claim 15, wherein the anti-inflammatory agent is an agent which inhibits the activity of RIPK1 kinase or RIPK3 kinase.

22. (canceled)

23. (canceled)

24. The method as claimed in claim 1, or claim 15, wherein the anti-inflammatory agent is an inhibitor of inflammasome activation.

25. (canceled)

26. The method as claimed in claim 1, wherein the anti-inflammatory agent is nonsteroidal anti-inflammatory drug (NSAID) optionally selected from aspirin, inhibitors of COX-1 and/or COX-2, ibuprofen, paracetamol.

27. The method as claimed in claim 1, wherein the anti-inflammatory agent is steroidal anti-inflammatory drug (SAID), optionally selected from triamcinolone, prednisone, prednisolone, methyl-prednisolone, dexamethasone, hydrocortisone, cortisol and/or related corticosteroids.

28.-37. (canceled)

38. The method as claimed in claim 1, wherein the combination comprises a TRAIL-inhibiting agent, a FasL-inhibiting agent; a TNF- and/or TNF/LT-alpha-inhibiting agent.

39. The method as claimed in claim 1, wherein the combination comprises a TRAIL-inhibiting agent and an inhibitor of type I and/or type II IFN signalling, which is optionally an inhibitor of a JAK1/2 kinase.

40.-44. (canceled)

45. The method as claimed in claim 1, wherein
(i) the organ is the pancreas, and the treatment is for diabetes;
(ii) the organ is the heart, and the treatment is for chronic heart disease;
(iii) the organ is the liver, and the treatment is for liver fibrosis;
(iv) the organ is the brain, and the treatment is for loss of motor or cognitive brain function;
(v) the organ is the lung, and the treatment is for acute or chronic failure of and/or damage to the lung; or
(vi) the treatment is for ARDS.

46. A method for treating a subject for a disease caused or induced by an airway-targeting virus, the method comprising administering to the subject a combination treatment of at least 2 agents, the combination comprising:
(a) one or more agents selected from the list consisting of:
(i) a FasL-inhibiting agent, and/or
(ii) a TRAIL-inhibiting agent,
(b) an anti-inflammatory agent.

47. A method of enhancing the therapeutic effectiveness of:
(a) one or more agents selected from the list consisting of:
(i) a FasL-inhibiting agent, and/or
(ii) a TRAIL-inhibiting agent,
for treating a subject for a disease caused or induced by an airway targeting virus the method comprising administering to the subject
(b) an anti-inflammatory agent.

48.-54. (canceled)

55. The method as claimed in claim 1 for treating ARDS, wherein ARDS is induced by the airway targeting virus.

56. The method as claimed in claim 55, wherein the airway targeting virus is selected from Influenza A virus; Influenza B virus; Avian influenza H5N1 or H7N9 viruses; Parainfluenza virus; Human metapneumovirus virus; Respiratory syncytial virus; Rhinovirus; Coronavirus; Hantavirus; Measles virus; Adenovirus; Human adenovirus type 55; Varicella-zoster virus; Cytomegalovirus and Herpes simplex virus,
wherein the coronavirus is optionally selected from SARS-CoV, SARS-CoV2, MERS-CoV.

57. (canceled)

58. (canceled)

59. The method as claimed in claim 1, wherein if the agent inhibits a receptor or ligand, it prevents or inhibits the ligand from binding to the receptor or disrupts the receptor/ligand complex resulting from binding.

60.-64. (canceled)

65. The method as claimed in claim 1, wherein if the agent is a FasL-inhibiting agent, it is Asunercept or an anti-FasL antibody.

66.-78. (canceled)


SEQUENCE LISTING (2 sequences)

SEQ ID NO: 1 - protein, 435 aa, Artificial Sequence
  synthetic construct
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 7 aa, Artificial Sequence
  synthetic construct
ITQQDLA